(19) United States
(12) Patent Application Publication
(10) Pub. No.: US 2008/0187575 A1
(43) Pub. Date: Aug. 7, 2008

(54) Pyrimidine Derivatives

(76) Inventors: Bert Klebl, Oberschweinbach (DE); Matthias Baumann, Munich (DE); Edmund Hoppe, Krailling (DE); Dirk Brehmer, Grafelfing (DE); Henrik Daub, Munich (DE); Gyorgy Keri, Budapest (HU); Zoltan Varga, Budapest (HU); Jeno Marosfalvi, Budapest (HU); Laszlo Orfi, Budapest (HU)

Correspondence Address: Leon R. Yankwich;Yankwich & Associtaes, 201 Broadway, Cambridge, MA, 02139, US

(21) Appl. No.: 11/661,041

(22) PCT Filed: Aug. 29, 2005
(86) PCT No.: PCT/EP2005/009291

§ 371 Date: Sep. 24, 2007

Related U.S. Application Data
(60) Provisional application No. 60/604,685, filed on Aug. 27, 2004.

(51) Int. Cl. A61K 9/127; C07D 239/42; A61K 31/505; C12Q 1/48; A61P 37/00; A61P 31/00; A61P 9/00; A61P 25/00; A61P 29/00; A61P 35/00; C07D 403/10; A61K 9/28

(52) U.S. Cl. 424/450; 544/332; 514/275; 544/331; 424/474; 435/15

(57) ABSTRACT

The present invention relates to pyrimidine derivatives, methods for their synthesis, and the use of said pyrimidine derivatives as pharmaceutically active agents, especially for the prophylaxis and/or treatment of cell proliferation disorders, cancer, leukemia, erectile dysfunction, cardiovascular diseases and disorders, inflammatory diseases, transplant rejection, immunological diseases, neuroimmunological diseases, autoimmune diseases, infective diseases including opportunistic infections, prion diseases and/or neuro-degeneration. Furthermore, the present invention relates to pharmaceutical compositions containing at least one pyrimidine derivative and/or pharmaceutically acceptable salts thereof as an active ingredient together with at least one pharmaceutically acceptable carrier, excipient or diluents as well as to methods for prophylaxis and/or treatment of the above-mentioned diseases and disorders.

The present invention relates to pyrimidine derivatives, methods for their synthesis, and the use of said pyrimidine derivatives as pharmaceutically active agents, especially for the prophylaxis and/or treatment of cell proliferation disorders, cancer, leukemia, erectile dysfunction, cardiovascular diseases and disorders, inflammatory diseases, transplant rejection, immunological diseases, neuroimmunological diseases, autoimmune diseases, infective diseases including opportunistic infections, prion diseases and/or neuro-degeneration. Furthermore, the present invention relates to pharmaceutical compositions containing at least one pyrimidine derivative and/or pharmaceutically acceptable salts thereof as an active ingredient together with at least one pharmaceutically acceptable carrier, excipient or diluents as well as to methods for prophylaxis and/or treatment of various diseases and disorders.

It is object of the present invention to provide novel compounds which can be used as pharmaceutically active agents, especially for prophylaxis and/or treatment of several diseases such as cell proliferation disorders, cancer, leukemia, erectile dysfunction, cardiovascular diseases and disorders, inflammatory diseases, transplant rejection, immunological diseases, neuroimmunological diseases, autoimmune diseases, infective diseases including opportunistic infections, prion diseases, neurodegenerative disorders, and/or neuro-degeneration as well as pharmaceutical compositions containing at least one of said novel compounds as active ingredient.

The object of the present invention is solved by the teaching of the independent claims. Further advantageous features, aspects and details of the invention are evident from the dependent claims, the description, and the examples of the present application.

One aspect of the present invention is related to compounds of the general formula (I):

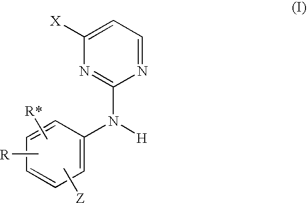

wherein:

R and R* represent independently of each other —H, —OCH₃, —CF₃, —CH₃, —C₂H₅, —R′, —R¹⁷;

R′, R″, R′″ and R″″ represent independently of each other —H, —F, —Cl, —Br, —I, —CN, —OH, —OCH₃, —OC₂H₅, —OCF₃, —NH₂, —NO₂, —N(CH₃)₂, —N(C₂H₅)₂, —SH, —SO₃H, —COOH, —COOCH₃, —COOC₂H₅, —CONH₂;

R¹, R², R³, R⁴, R¹′, R²′, and R³′ represent independently of each other —H, —R′, —OH, —SH, —OCH₃, —OC₂H₅, —SCH₃, —NH₂, —NO₂, —NH(CH₃), —N(CH₃)₂, —COOH, —COOCH₃, —OCF₃, —CH₃, —C₂H₅, —C₃H₇, —CH(CH₃)₂, —R¹²,

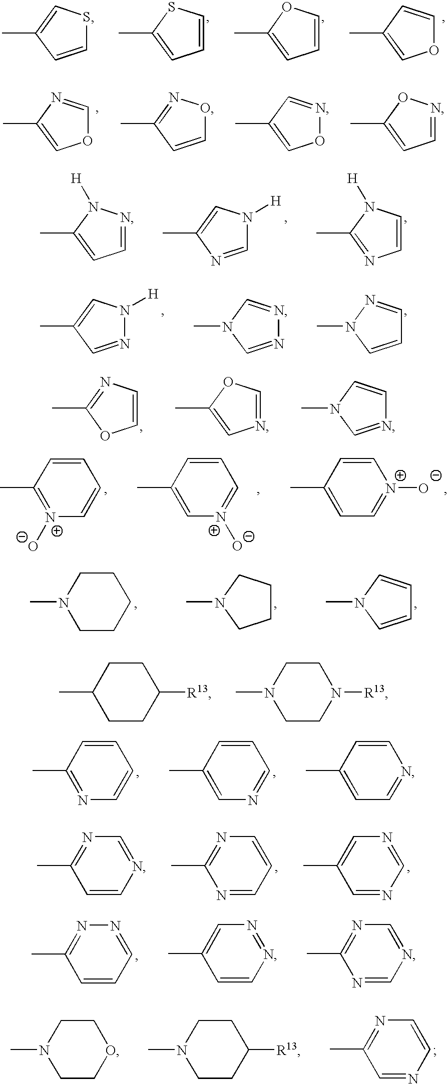

R⁵ represents —H, —R⁴, —CH₂R³, —C₂H₄R³, —C₃H₆R³, —C₄H₈R³, —CHR³R⁴, —CH₂—CHR³R⁴, —C₂H₄—CHR³R⁴, —C₃H₆—CHR³R⁴, —R¹¹, —R¹³,

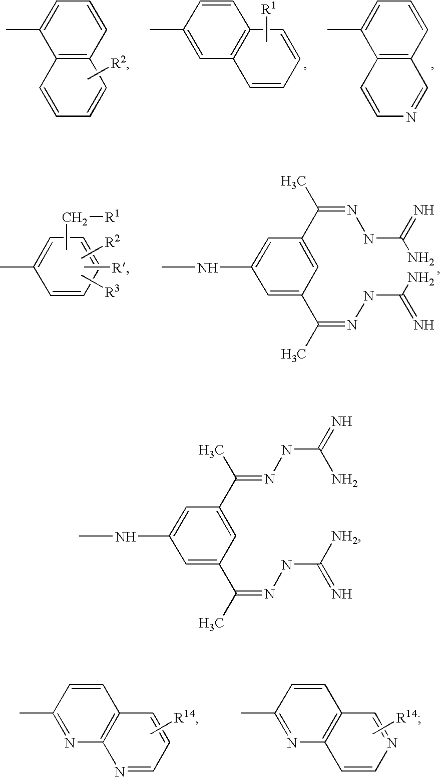

R⁶, R⁷, R⁸ and R⁹ represent independently of each other —H, —R′, —R¹, —CH₂R¹, —R¹²;

R¹⁰, R¹¹, R¹⁷, R¹⁸ and R¹⁹ represent independently of each other —H, —R′, —CH₃, —C₂H₅, —CH═CH₂, —C≡CH, —C₃H₇, -cyclo-C₃H₅, —CH(CH₃)₂, —CH₂—CH═CH₂, —C(CH₃)═CH₂, —CH═CH—CH₃, —C≡C—CH₃, —CH₂—C≡CH, —C₄H₉, -cyclo-C₄H₇, —CH₂—CH(CH₃)₂, —CH(CH₃)—C₂H₅, —C(CH₃)₃, —C₅H₁₁, -cyclo-C₅H₉, —C₆H₁₃, -cyclo-C₆H₁₁, -Ph, —C(R′)₃, —CR′(R″)₂, —CR′(R″)R′″, —C₂(R′)₅, —CH₂—C(R′)₃, —CH₂—CR′(R″)₂, —CH₂—CR′(R″)R′″, —C₂H₄—C(R′)₃, —CH(R′)—CH(R″)—CH₂—R′″, —C₃(R′)₇, —CH₂—R′, —C₂H₄—R′, —C₃H₆—R′, —C₄H₈—R′, —C₅H₁₀—R′, —C₆H₁₂—R′;

R¹² and R¹³ represent independently of each other —H, —F, —Cl, —Br, —I, —CH₂F, —CH₂Cl, —CH₂Br, —CH₂I, —CH₂R³, —OH, —OCH₃, —OC₂H₅, —NH₂, —NH(CH₃), —N(CH₃)₂, —N(C₂H₅)₂, —OCF₃, —CH₃, —C₂H₅, —C₃H₇, —R¹⁰, —NH(R¹⁰)—NH(R¹¹), —N(R¹⁰)₂, —NR¹⁰R¹¹, —OR¹⁰, —OR¹¹, —CO—R¹⁰, —COOH, —COOCH₃, —COOC₂H₅, —COOR¹⁰, —OOCR¹⁰, —SO₃H, —SO₃R¹⁰, —SO₂H, —SO₂R¹⁰, —SO₂—CH₃, —CO—CH₃, —OOC—CH₃, —OOC—C₂H₅, —CONH₂, —CONH(R¹⁰), —CON(R¹⁰)₂, —CONR¹⁰R¹¹, —NH—CO—R¹⁰, —NH—CO—CH₃, —NH—CO—C₂H₅, —NH—CO—C(CH₃)₃, —NH—CO—OCH₃, —NH—CO—NH₂;

R¹⁴ and R¹⁵ represent independently of each other —H, —R¹′, —F, —Cl, —Br, —I, —CH₂F, —CH₂Cl, —CH₂Br, —CH₂I, —CH₂R³, —OH, —OCH₃, —OC₂H₅, —NH₂, —NH(CH₃), —N(CH₃)₂, —N(C₂H₅)₂, —OCF₃, —CH₃, —C₂H₅, —C₃H₇, —R¹⁸, —NH(R¹⁸), —NH(R¹⁹), —N(R¹⁸)₂, —NR¹⁸R¹⁹, —OR¹⁸, —CO—R¹⁸, —COOH, —COOCH₃, —COOC₂H₅, —COOR¹⁸, —OOCR¹⁸, —SO₃H, —SO₃R¹⁸, —SO₂H, —SO₂R¹⁸, —SO₂—CH₃, —CO—CH₃, —OOC—CH₃, —OOC—C₂H₅, —CONH₂, —CONH(R¹⁸), —CON(R¹⁸)₂, —CONR¹⁸R¹⁹, —NH—CO—R¹⁸, —NH—CO—CH₃, —NH—CO—C₂H₅, —NH—CO—C(CH₃)₃, —NH—CO—OCH₃, —NH—CO—NH₂, —CR¹′(R²′)R³′, —CH₂—CR¹′(R²′)R³′, —CHR¹′—CH₂R²′, —CH(R¹′)—CH(R²′)—CH₂—R³′, —CH₂—R¹′, —C₂H₄—R¹′, —C₃H₆—R¹′, —C₄H₈—R¹′, —C₅H₁₀—R¹′, —C₆H₁₂—R¹′;

X represents

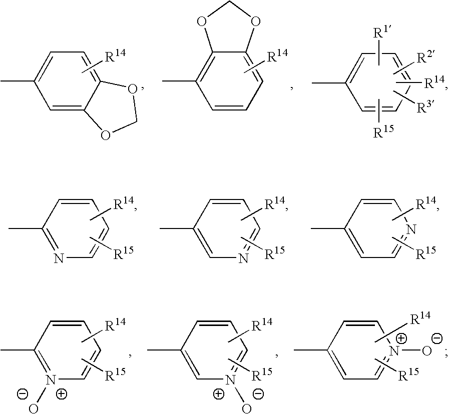

Z represents —NH—CO—R⁵, —CO—NH—R⁵, —NH—CS—R⁵, —NH—SO₂—R⁵, —NH₂, —NO₂, —OCH₃, —SCH₃, —CF₃, —COOH, —COOCH₃, —COOC₂H₅,

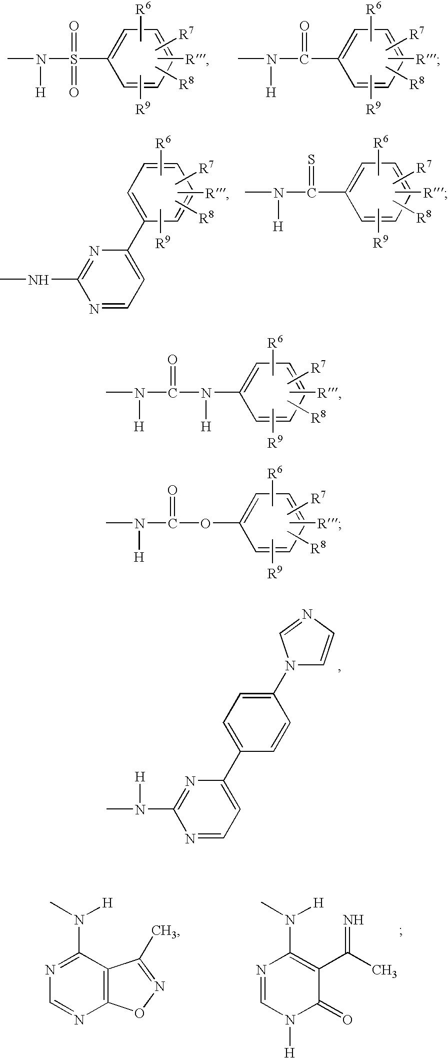

and/or pharmaceutically acceptable salts thereof;

excluded are the following compounds,
1-(4-{4-[4-(4-Imidazol-1-ylphenyl)-pyrimidin-2-ylamino]-benzoyl}-piperazin-1-yl)ethanone,
4-[4-(4-Imidazol-1-ylphenyl)-pyrimidin-2-ylamino]-benzamide,
2-(3-Fluorophenylamino)-4-(4-imidazol-1-ylphenyl)-pyrimidine-5-carbonitrile.

Another aspect of the present invention relates compounds of the general formula (I) wherein

R represents —H, —CH₃, —C₂H₅, —R′, —R¹⁰, —R¹²;

R* represents —H;

R′, R″ and R′″ represent independently of each other —H, —F, —Cl, —Br, —I, —CN;

R¹, R², R³ and R⁴ represent independently of each other —H, —R′, —OH, —OCH₃, —NH₂, —NO₂, —N(CH₃)₂, —COOH, —COOCH₃, —OCF₃, —CH₃, —C₂H₅, —OC₂H₅, —R¹²,

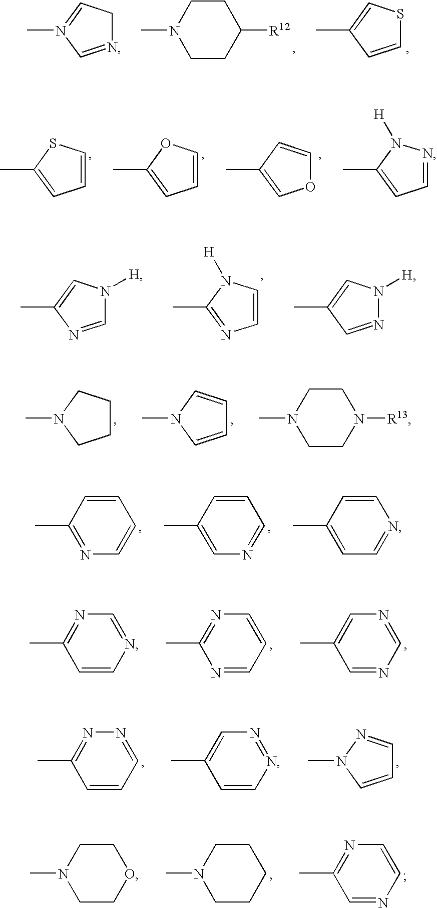

R⁵ represents —H, —R⁴, —CH₂R³, —C₂H₄R³, —C₃H₆R³, —R¹¹, —R¹³,

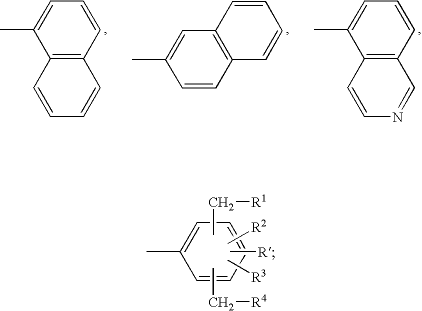

R⁶, R⁷, R⁸ and R⁹ represent independently of each other —H, —R′, —R¹, —CH₂R¹, R¹²;

R¹⁰ and R¹¹ represent independently of each other —H, —CH₃, —C₂H₅, —CH═CH₂, —C≡CH, —C₃H₇, -cyclo-C₃H₅, —CH(CH₃)₂, —CH₂—CH═CH₂, —CH═CH—CH₃, —C≡C—CH₃, —CH₂—C≡CH, —C₄H₉, —CH₂—CH(CH₃)₂, —C(CH₃)₃, —C₅H₁₁, -cyclo-C₅H₉, —C₆H₁₃, -cyclo-C₆H₁₁, -Ph, —C(R′)₃, —CH₂—C(R′)₃, —CH₂—C(R″)₃;

R¹² and R¹³ represent independently of each other —CH₂F, —CH₂Cl, —CH₂Br, —CH₂I, —CH₂R³, —OH, —OCH₃, —NH₂, —NH(CH₃), —N(CH₃)₂, —OCF₃, —CH₃, —R¹⁰, —N(R¹⁰)₂, —OR¹⁰, —COOH, —COOR¹⁰, —OOCR¹⁰, —CONH₂, —CON(R¹⁰)₂;

X is

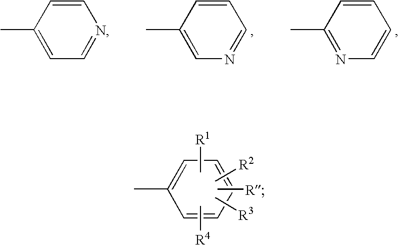

Z represents —NH—CO—R⁵,

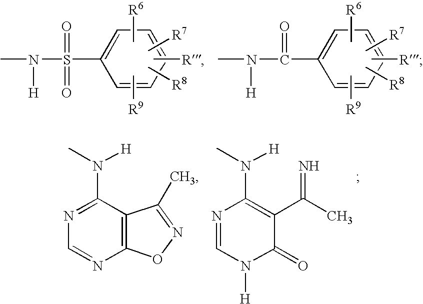

Also preferred are the compounds of the general formula (Ia) and (Ib)

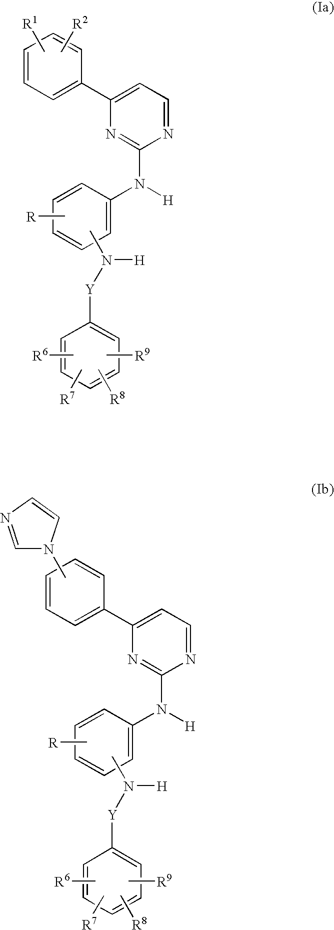

wherein the substituents R, R¹, R², R⁶, R⁷, R⁸, and R⁹ have the meanings as disclosed above and Y represents the residue —C(═O) or —SO₂—.

The general formula (Ic) is also preferred

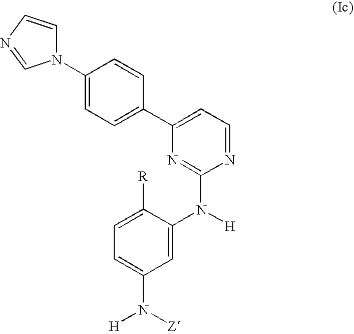

wherein

Z′ is

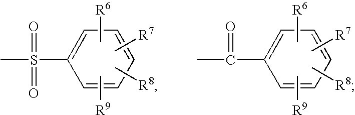

and the substituents R⁶, R⁷, R⁸, and R⁹ have the meanings as disclosed above.

Especially preferred are the following general formulas (Id) and (Ie)

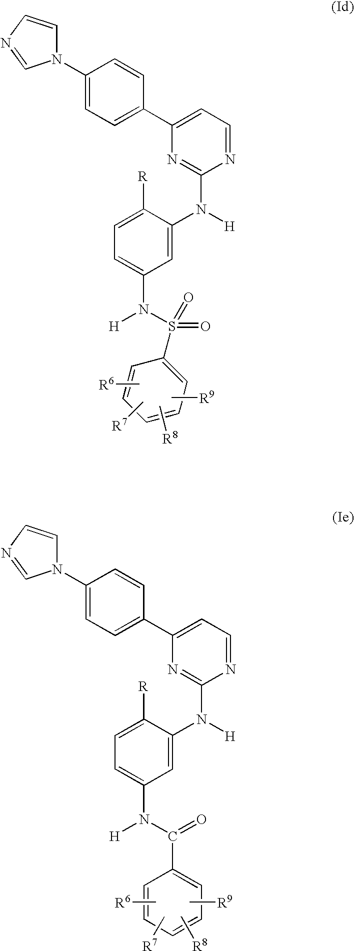

wherein the substituents R⁶, R⁷, R⁸, and R⁹ have the meanings as disclosed above.

Within the formulas (I), (Ia), (Ib), (Ic), (Id), and (Ie) the residue R is preferably hydrogen or a methyl group. Furthermore, R is preferably in ortho position to the pyrimidinylamino group and/or in para position to the amino carbonyl group. Consequently, the amino carbonyl group is preferably in meta position to the pyrimidinylamino residue. Nevertheless, the para position of the amino carbonyl group to the pyrimidinylamino residue is also preferred.

Preferred is still another subformula of general formula (I). Said formula (If) is represented by the following structure

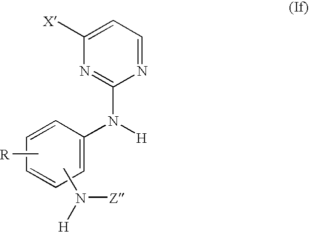

wherein

X′ represents one of the following residues

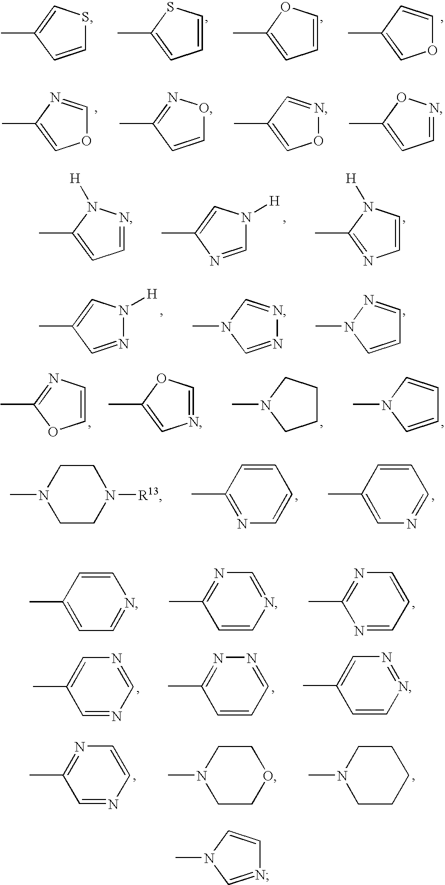

and Z″ represents —CO—R⁷, —CO—R¹², —SO₂—R⁷, —SO₂—R¹²; and R, R⁷, and R¹² have the meanings as disclosed above.

General formula (Ig) is another preferred subformula

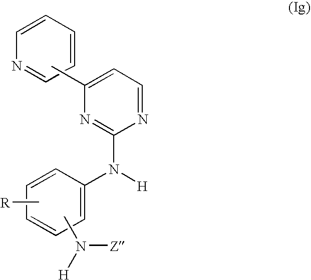

wherein

Z″ represents —CO—R⁷, —CO—R¹², —SO₂—R⁷, —SO₂—R¹²;

and R, R⁷, and R¹² have the meanings as disclosed above.

Preferred is still the general formula (Ih)

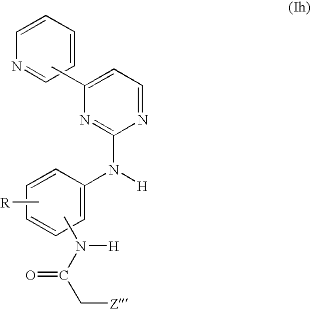

wherein

Z′″ represents —R¹, —R⁵, and —R¹³, provided that Z′″ is not —H or CₙH₂ₙ₊₁ with n being an integer between 1 and 6, and

wherein the substituents R, R¹, R⁵, and R¹³ have the meanings as disclosed above.

Within the formulas (If), (Ig), and (Ih) the residue R is preferably hydrogen or a methyl group. Furthermore, R is preferably in ortho position to the pyrimidinylamino group and/or in para position to the amino carbonyl group. Consequently, the amino carbonyl group is preferably in meta position to the pyrimidinylamino residue. Nevertheless, the para position of the amino carbonyl group to the pyrimidinylamino residue is also preferred.

Another preferred formula (Ik) is represented by the structure

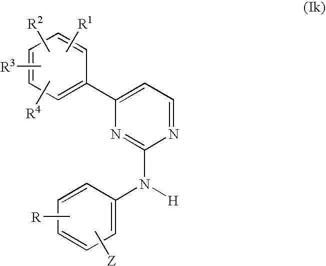

wherein

Z, R, R¹, R², R³, and R⁴ have the meanings as disclosed above.

Also preferred is the following general formula (Im)

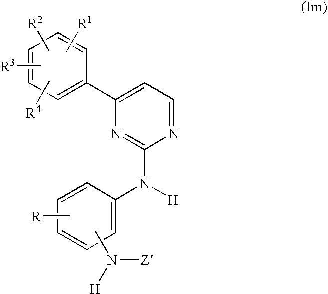

wherein

at least two of the substituents R¹, R², R³, and R⁴ are different from hydrogen, R, R¹, R², R³, and R⁴ have the meanings as disclosed above and

Z′ is

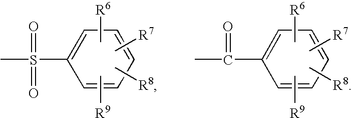

Within formula (Im) substituents R¹, R², R³, and R⁴ are preferred which comprise a heteroatom and more preferably comprise oxygen or nitrogen and most preferably comprise oxygen.

Thus, the following general formula (In) represents preferred compounds

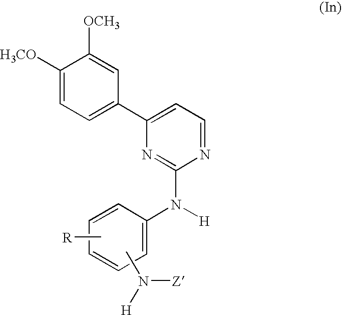

wherein

R and Z′ have the meanings as disclosed above.

Within the formulas (Ik), (Im), and (In) the residue R is preferably hydrogen or a methyl group. Furthermore, R is preferably in ortho position to the pyrimidinylamino group and/or in para position to the —NHZ′ group and the -Z group respectively. Consequently, the —NHZ′ or the -Z group is preferably in meta position to the pyrimidinylamino residue. Nevertheless, the para position of the —NHZ′ or the -Z group to the pyrimidinylamino residue is also preferred.

Another group of preferred compounds is obtained in the case wherein X represents a pyridyl residue and Z is one of the following residues

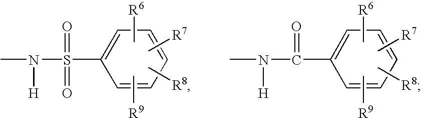

The para position of the residue Z is preferred.

Other preferred compounds are represented by the general formula (Io)

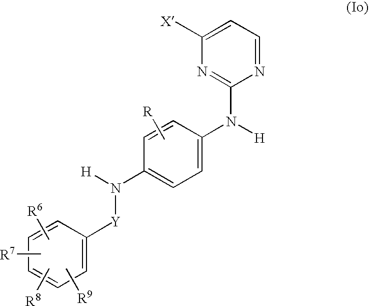

wherein

X′, Y, R, R⁶, R⁷, R⁸, and R⁹ have the meanings as disclosed above, provided that x is not 3-pyridyl.

Further preferred are compounds represented by the general formula (Io), wherein X′ is 2-pyridyl or 4-pyridyl.

Within formula (Io) it is preferred that the substituent R represents hydrogen. Furthermore, heterocyclic substituents and especially heteroaromatic substituents are preferred as residue X′.

Thus, the following two general formulas (Ip) and (Iq) are preferred

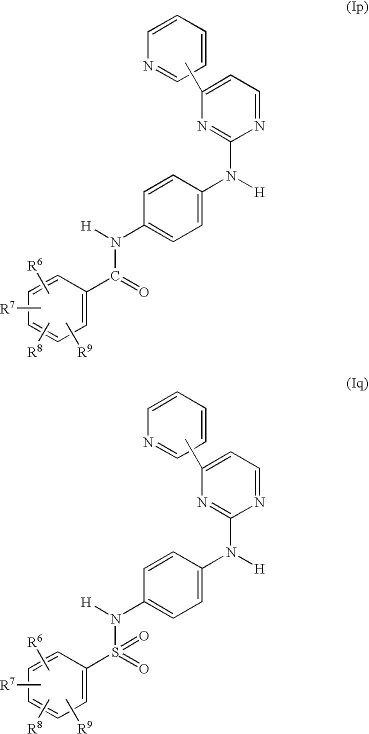

wherein

R⁶, R⁷, R⁸, and R⁹ have the meanings as disclosed above, provided that x is not 3-pyridyl.

Still another preferred general formula (Ir) is

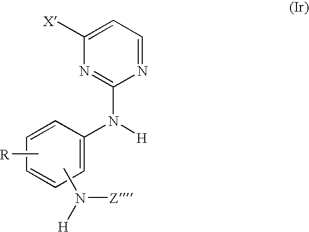

wherein

X′ and R have the meanings as disclosed above and

Z″″ is

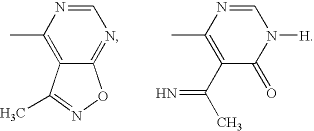

Within formula (Ir), (Is), and (It) it is preferred that the substituent R represents hydrogen or a methyl group. Furthermore, heterocyclic substituents and especially heteroaromatic substituents are preferred as residue X′.

Thus, the following two general formulas (Is) and (It) are preferred

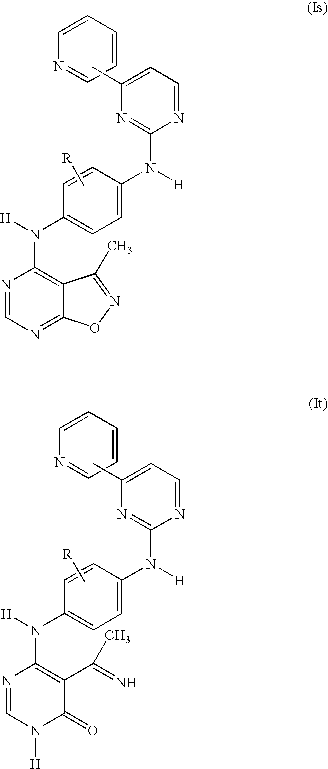

wherein

R has the meanings as disclosed above.

A further preferred subgroup of inventive compounds can be represented by the following general formulas (Iu), (Iv), (Iw), and (Ix) as shown below:

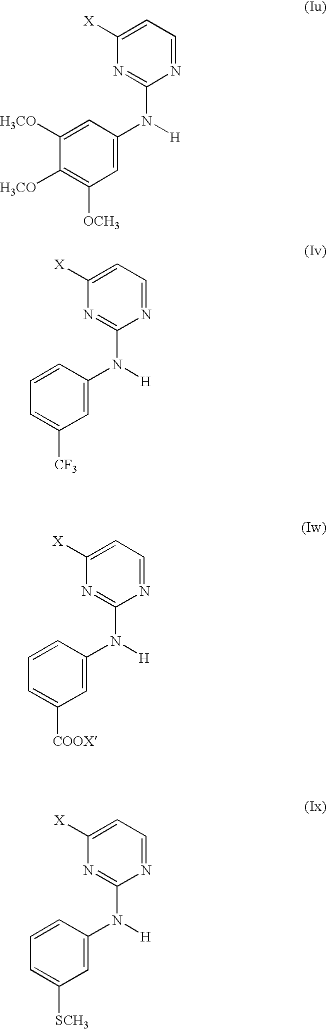

wherein

X has the meanings as described in general formula (I) and X′ represents —H, —CH₃, or —C₂H₅.

The pyrimidine compounds of the present invention are basic and form pharmaceutically acceptable salts with organic and inorganic acids. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, D-o-tolyltartaric acid, tartronic acid, α-toluic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner.

It is also possible to obtain acid addition salts with amino acids like methionine, tryptophane, lysine or arginine, especially with pyrimidine compounds of the general formula (I), (Ia)-(Ix) bearing a carboxylic acid residue.

Depending on the substituents of the inventive pyrimidine compounds, one may be able to form salts with bases, too. Thus, for example, if there are carboxylic acid substituents or other acidic residues in the molecule, salts may be formed with inorganic as well as organic bases such as, for example, LiOH, NaOH, KOH, CaCO₃, NH₄OH, tetraalkylammonium hydroxide, and the like.

Most preferred are compounds 58, 93, 96 to 291, and 294 to 312 selected from the list of compounds below, and salts thereof:

### Compound List:

Compound 1 (2-Methyl-5-nitro-phenyl)-(4-pyridin-2-yl-pyrimidin-2-yl)-amine
Compound 2 (3-Nitro-phenyl)-(4-pyridin-3-yl-pyrimidin-2-yl)-amine
Compound 3 N-(4-Pyridin-3-yl-pyrimidin-2-yl)-benzene-1,3-diamine
Compound 4 4-Methyl-N-3-(4-pyridin-3-yl-pyrimidin-2-yl)-benzene-1,3-diamine
Compound 5 4-Chloromethyl-N-[4-methyl-3-(4-pyridin-3-yl-pyrimidin-2-ylamino)-phenyl]-benzamide
Compound 6 4-Chloromethyl-N-[3-(4-pyridin-3-yl-pyrimidin-2-ylamino)-phenyl]-benzamide
Compound 7 4-(4-Methyl-piperazin-1-ylmethyl)-N-[3-(4-pyridin-3-yl-pyrimidin-2-ylamino)-phenyl]-benzamide
Compound 8 4-(4-Methyl-piperazin-1-ylmethyl)-N-[4-methyl-3-(4-pyridin-3-yl-pyrimidin-2-ylamino)-phenyl]-benzamide
Compound 9 4-Chloro-N-[4-methyl-3-(4-pyridin-3-yl-pyrimidin-2-ylamino)-phenyl]-benzamide
Compound 10 4-Chloro-N-[3-(4-pyridin-3-yl-pyrimidin-2-ylamino)-phenyl]-benzamide
Compound 11 3,4,5-Trimethoxy-N-[3-(4-pyridin-3-yl-pyrimidin-2-ylamino)-phenyl]-benzamide
Compound 12 4-Cyano-N-[3-(4-pyridin-3-yl-pyrimidin-2-ylamino)-phenyl]-benzamide
Compound 13 4-Methoxy-N-[3-(4-pyridin-3-yl-pyrimidin-2-ylamino)-phenyl]-benzamide
Compound 14 4-Chloro-N-[3-(4-pyridin-3-yl-pyrimidin-2-ylamino)-phenyl]-benzenesulfonamide
Compound 15 Thiophene-3-carboxylic acid [3-(4-pyridin-3-yl-pyrimidin-2-ylamino)-phenyl]-amide
Compound 16 3,5-Dimethoxy-N-[3-(4-pyridin-3-yl-pyrimidin-2-ylamino)-phenyl]-benzamide
Compound 17 3,4,5-Trimethoxy-N-[4-methyl-3-(4-pyridin-3-yl-pyrimidin-2-ylamino)-phenyl]-benzamide
Compound 18 4-Cyano-N-[4-methyl-3-(4-pyridin-3-yl-pyrimidin-2-ylamino)-phenyl]-benzamide
Compound 19 4-Methoxy-N-[4-methyl-3-(4-pyridin-3-yl-pyrimidin-2-ylamino)-phenyl]-benzamide
Compound 20 4-Chloro-N-[4-methyl-3-(4-pyridin-3-yl-pyrimidin-2-ylamino)-phenyl]-benzenesulfonamide
Compound 21 Thiophene-3-carboxylic acid [4-methyl-3-(4-pyridin-3-yl-pyrimidin-2-ylamino)-phenyl]-amide
Compound 22 3,5-Dimethoxy-N-[4-methyl-3-(4-pyridin-3-yl-pyrimidin-2-ylamino)-phenyl]-benzamide
Compound 23 N-[4-Methyl-3-(4-pyridin-3-yl-pyrimidin-2-ylamino)-phenyl]-4-trifluoromethoxy-benzamide
Compound 24 Cyclohexanecarboxylic acid [4-methyl-3-(4-pyridin-3-yl-pyrimidin-2-ylamino)-phenyl]-amide
Compound 25 Cyclohexanecarboxylic acid [3-(4-pyridin-3-yl-pyrimidin-2-ylamino)-phenyl]-amide
Compound 26 Isoquinoline-5-sulfonic acid [4-methyl-3-(4-pyridin-3-yl-pyrimidin-2-ylamino)-phenyl]-amide
Compound 27 Isoquinoline-5-sulfonic acid [3-(4-pyridin-3-yl-pyrimidin-2-ylamino)-phenyl]-amide
Compound 28 4-Methyl-N-3-(4-pyridin-2-yl-pyrimidin-2-yl)-benzene-1,3-diamine
Compound 29 4-Cyano-N-[4-methyl-3-(4-pyridin-4-yl-pyrimidin-2-ylamino)-phenyl]-benzamide
Compound 30 4-Chloro-N-[4-methyl-3-(4-pyridin-4-yl-pyrimidin-2-ylamino)-phenyl]-benzenesulfonamide
Compound 31 4-Methoxy-N-[4-methyl-3-(4-pyridin-4-yl-pyrimidin-2-ylamino)-phenyl]-benzamide
Compound 32 4-Chloro-N-[4-methyl-3-(4-pyridin-4-yl-pyrimidin-2-ylamino)-phenyl]-benzamide
Compound 33 Cyclohexanecarboxylic acid [3-(4-pyridin-4-yl-pyrimidin-2-ylamino)-phenyl]-amide
Compound 34 3,5-Dimethoxy-N-[3-(4-pyridin-4-yl-pyrimidin-2-ylamino)-phenyl]-benzamide
Compound 35 4-Methyl-N-3-(4-pyridin-4-yl-pyrimidin-2-yl)-benzene-1,3-diamine
Compound 36 Thiophene-3-carboxylic acid [3-(4-pyridin-4-yl-pyrimidin-2-ylamino)-phenyl]-amide
Compound 37 4-Chloro-N-[3-(4-pyridin-4-yl-pyrimidin-2-ylamino)-phenyl]-benzenesulfonamide
Compound 38 4-Chloro-N-[3-(4-pyridin-4-yl-pyrimidin-2-ylamino)-phenyl]-benzamide
Compound 39 N-(4-Pyridin-4-yl-pyrimidin-2-yl)-benzene-1,3-diamine
Compound 40 (3-Nitro-phenyl)-(4-pyridin-4-yl-pyrimidin-2-yl)-amine
Compound 41 N-[4-Methyl-3-(4-pyridin-4-yl-pyrimidin-2-ylamino)-phenyl]-4-trifluoromethoxy-benzamide
Compound 42 Isoquinoline-5-sulfonic acid [3-(4-pyridin-4-yl-pyrimidin-2-ylamino)-phenyl]-amide
Compound 43 4-Methoxy-N-[3-(4-pyridin-4-yl-pyrimidin-2-ylamino)-phenyl]-benzamide
Compound 44 4-Cyano-N-[3-(4-pyridin-4-yl-pyrimidin-2-ylamino)-phenyl]-benzamide
Compound 45 3,4,5-Trimethoxy-N-[3-(4-pyridin-4-yl-pyrimidin-2-ylamino)-phenyl]-benzamide
Compound 46 3,5-Dimethoxy-N-[4-methyl-3-(4-pyridin-4-yl-pyrimidin-2-ylamino)-phenyl]-benzamide
Compound 47 3,4,5-Trimethoxy-N-[4-methyl-3-(4-pyridin-4-yl-pyrimidin-2-ylamino)-phenyl]-benzamide
Compound 48 Thiophene-3-carboxylic acid [4-methyl-3-(4-pyridin-4-yl-pyrimidin-2-ylamino)-phenyl]-amide
Compound 49 3,4,5-Trimethoxy-N-[4-methyl-3-(4-pyridin-2-yl-pyrimidin-2-ylamino)-phenyl]-benzamide
Compound 50 4-Cyano-N-[4-methyl-3-(4-pyridin-2-yl-pyrimidin-2-ylamino)-phenyl]-benzamide
Compound 51 N-(4-Pyridin-2-yl-pyrimidin-2-yl)-benzene-1,3-diamine
Compound 52 4-Chloro-N-[4-methyl-3-(4-pyridin-2-yl-pyrimidin-2-ylamino)-phenyl]-benzamide
Compound 53 Cyclohexanecarboxylic acid [4-methyl-3-(4-pyridin-2-yl-pyrimidin-2-ylamino)-phenyl]-amide
Compound 54 4-Methyl-N-[4-methyl-3-(4-pyridin-2-yl-pyrimidin-2-ylamino)-phenyl]-benzenesulfonamide
Compound 55 4-Methoxy-N-[4-methyl-3-(4-pyridin-2-yl-pyrimidin-2-ylamino)-phenyl]-benzamide
Compound 56 3,5-Dimethoxy-N-[4-methyl-3-(4-pyridin-2-yl-pyrimidin-2-ylamino)-phenyl]-benzamide
Compound 57 Naphthalene-2-carboxylic acid [4-methyl-3-(4-pyridin-3-yl-pyrimidin-2-ylamino)-phenyl]-amide
Compound 58 [4-(4-Imidazol-1-yl-phenyl)-pyrimidin-2-yl]-(2-methyl-5-nitro-phenyl)-amine
Compound 59 4-Chloro-N-[3-(4-pyridin-2-yl-pyrimidin-2-ylamino)-phenyl]-benzamide
Compound 60 4-Methoxy-N-[3-(4-pyridin-2-yl-pyrimidin-2-ylamino)-phenyl]-benzamide
Compound 61 4-Chloro-N-[3-(4-pyridin-2-yl-pyrimidin-2-ylamino)-phenyl]-benzenesulfonamide
Compound 62 Thiophene-2-carboxylic acid [3-(4-pyridin-2-yl-pyrimidin-2-ylamino)-phenyl]-amide
Compound 63 Naphthalene-2-sulfonic acid [3-(4-pyridin-2-yl-pyrimidin-2-ylamino)-phenyl]-amide
Compound 64 Isoquinoline-5-sulfonic acid [3-(4-pyridin-2-yl-pyrimidin-2-ylamino)-phenyl]-amide
Compound 65 Cyclopentanecarboxylic acid [3-(4-pyridin-2-yl-pyrimidin-2-ylamino)-phenyl]-amide
Compound 66 Naphthalene-2-carboxylic acid [3-(4-pyridin-2-yl-pyrimidin-2-ylamino)-phenyl]-amide
Compound 67 4-Cyano-N-[3-(4-pyridin-2-yl-pyrimidin-2-ylamino)-phenyl]-benzamide
Compound 68 3,5-Dimethoxy-N-[3-(4-pyridin-2-yl-pyrimidin-2-ylamino)-phenyl]-benzamide
Compound 69 4-Bromo-N-[3-(4-pyridin-2-yl-pyrimidin-2-ylamino)-phenyl]-benzamide
Compound 70 4-Methyl-N-[3-(4-pyridin-2-yl-pyrimidin-2-ylamino)-phenyl]-benzamide
Compound 71 4-Fluoro-N-[3-(4-pyridin-2-yl-pyrimidin-2-ylamino)-phenyl]-benzenesulfonamide
Compound 72 3,5-Dichloro-N-[3-(4-pyridin-2-yl-pyrimidin-2-ylamino)-phenyl]-benzamide
Compound 73 N-[3-(4-Pyridin-2-yl-pyrimidin-2-ylamino)-phenyl]-benzamide
Compound 74 4-Chloromethyl-N-[3-(4-pyridin-2-yl-pyrimidin-2-ylamino)-phenyl]-benzamide
Compound 75 4-Methyl-N-[3-(4-pyridin-2-yl-pyrimidin-2-ylamino)-phenyl]-benzenesulfonamide
Compound 76 4-(4-Methyl-piperazin-1-ylmethyl)-N-[3-(4-pyridin-2-yl-pyrimidin-2-ylamino)-phenyl]-benzamide
Compound 77 Naphthalene-2-carboxylic acid [3-(4-pyridin-4-yl-pyrimidin-2-ylamino)-phenyl]-amide
Compound 78 2-Methoxy-N-[4-methyl-3-(4-pyridin-3-yl-pyrimidin-2-ylamino)-phenyl]-benzamide
Compound 79 N-[4-(4-Pyridin-3-yl-pyrimidin-2-ylamino)-phenyl]-benzamide
Compound 80 2-Methoxy-N-[3-(4-pyridin-3-yl-pyrimidin-2-ylamino)-phenyl]-benzamide
Compound 81 4-Methyl-N-[3-(4-pyridin-3-yl-pyrimidin-2-ylamino)-phenyl]-benzamide
Compound 82 4-Methyl-N-[4-methyl-3-(4-pyridin-3-yl-pyrimidin-2-ylamino)-phenyl]-benzamide
Compound 83 N-[4-Methyl-3-(4-pyridin-3-yl-pyrimidin-2-ylamino)-phenyl]-benzamide
Compound 84 1-(3,5-Diacetyl-phenyl)-3-[4-methyl-3-(4-pyridin-3-yl-pyrimidin-2-ylamino)-phenyl]-urea
Compound 85 1-(3,5-Diacetyl-phenyl)-3-[4-methyl-3-(4-pyridin-3-yl-pyrimidin-2-ylamino)-phenyl]-urea-bis-aminohydrazone
Compound 86 N-[4-Methyl-3-(4-pyridin-3-yl-pyrimidin-2-ylamino)-phenyl]-nicotinamide
Compound 87 N-[3-(4-Pyridin-3-yl-pyrimidin-2-ylamino)-phenyl]-nicotinamide
Compound 88 [1,8]Naphthyridine-2-carboxylic acid [3-(4-pyridin-3-yl-pyrimidin-2-ylamino)-phenyl]-amide
Compound 89 [1,8]Naphthyridine-2-carbothioic acid [3-(4-pyridin-3-yl-pyrimidin-2-ylamino)-phenyl]-amide
Compound 90 2-Methoxy-N-[3-(4-pyridin-4-yl-pyrimidin-2-ylamino)-phenyl]-benzamide
Compound 91 N-(4-Pyridin-3-yl-pyrimidin-2-yl)-benzene-1,4-diamine
Compound 92 N-[3-(4-Pyridin-4-yl-pyrimidin-2-ylamino)-phenyl]-4-trifluoromethoxy-benzamide
Compound 93 4-Cyano-N-[4-(4-pyridin-3-yl-pyrimidin-2-ylamino)-phenyl]-benzamide
Compound 94 4-Methyl-N-[3-(4-pyridin-4-yl-pyrimidin-2-ylamino)-phenyl]-benzamide
Compound 95 N-[3-(4-Pyridin-4-yl-pyrimidin-2-ylamino)-phenyl]-benzamide
Compound 96 Naphthalene-2-carboxylic acid [4-(4-pyridin-3-yl-pyrimidin-2-ylamino)-phenyl]-amide
Compound 97 N-(4-Pyridin-2-yl-pyrimidin-2-yl)-benzene-1,4-diamine
Compound 98 3,4,5-Trimethoxy-N-[4-(4-pyridin-3-yl-pyrimidin-2-ylamino)-phenyl]-benzamide
Compound 99 Thiophene-2-sulfonic acid [4-(4-pyridin-3-yl-pyrimidin-2-ylamino)-phenyl]-amide
Compound 100 2-Methoxy-N-[4-(4-pyridin-3-yl-pyrimidin-2-ylamino)-phenyl]-benzamide
Compound 101 4-Methyl-N-[4-(4-pyridin-3-yl-pyrimidin-2-ylamino)-phenyl]-benzamide
Compound 102 [1,6]Naphthyridine-2-carboxylic acid [3-(4-pyridin-3-yl-pyrimidin-2-ylamino)-phenyl]-amide.
Compound 103 N-(4-Pyridin-4-yl-pyrimidin-2-yl)-benzene-1,4-diamine
Compound 104 [1,6]Naphthyridine-2-carbothioic acid [3-(4-pyridin-3-yl-pyrimidin-2-ylamino)-phenyl]-amide
Compound 105 4-Cyano-N-[4-(4-pyridin-4-yl-pyrimidin-2-ylamino)-phenyl]-benzamide
Compound 106 4-Chloromethyl-N-[4-(4-pyridin-4-yl-pyrimidin-2-ylamino)-phenyl]-benzamide
Compound 107 4-Chloromethyl-N-[4-(4-pyridin-3-yl-pyrimidin-2-ylamino)-phenyl]-benzamide
Compound 108 4-(4-Methyl-piperazin-1-ylmethyl)-N-[4-(4-pyridin-3-yl-pyrimidin-2-ylamino)-phenyl]-benzamide
Compound 109 4-Chloro-N-[4-(4-pyridin-3-yl-pyrimidin-2-ylamino)-phenyl]-benzamide
Compound 110 Naphthalene-2-carboxylic acid [4-(4-pyridin-2-yl-pyrimidin-2-ylamino)-phenyl]-amide
Compound 111 3,4,5-Trimethoxy-N-[4-(4-pyridin-2-yl-pyrimidin-2-ylamino)-phenyl]-benzamide
Compound 112 N-[4-(4-Pyridin-2-yl-pyrimidin-2-ylamino)-phenyl]-4-trifluoromethoxy-benzamide
Compound 113 4-Chloro-N-[4-(4-pyridin-2-yl-pyrimidin-2-ylamino)-phenyl]-benzamide
Compound 114 2-Methoxy-N-[4-(4-pyridin-2-yl-pyrimidin-2-ylamino)-phenyl]-benzamide
Compound 115 4-Methyl-N-[4-(4-pyridin-2-yl-pyrimidin-2-ylamino)-phenyl]-benzamide
Compound 116 Thiophene-2-sulfonic acid [4-(4-pyridin-2-yl-pyrimidin-2-ylamino)-phenyl]-amide
Compound 117 N-[4-(4-Pyridin-3-yl-pyrimidin-2-ylamino)-phenyl]-4-trifluoromethoxy-benzamide
Compound 118 N-[4-(4-Pyridin-2-yl-pyrimidin-2-ylamino)-phenyl]-nicotinamide
Compound 119 Thiophene-2-carboxylic acid [4-(4-pyridin-2-yl-pyrimidin-2-ylamino)-phenyl]-amide
Compound 120 N-[4-(4-Pyridin-4-yl-pyrimidin-2-ylamino)-phenyl]-benzamide
Compound 121 N-[4-(4-Pyridin-3-yl-pyrimidin-2-ylamino)-phenyl]-nicotinamide
Compound 122 4-Methoxy-N-[4-(4-pyridin-3-yl-pyrimidin-2-ylamino)-phenyl]-benzamide
Compound 123 3,5-Dimethoxy-N-[4-(4-pyridin-2-yl-pyrimidin-2-ylamino)-phenyl]-benzamide
Compound 124 3,5-Dimethoxy-N-[4-(4-pyridin-3-yl-pyrimidin-2-ylamino)-phenyl]-benzamide
Compound 125 2-Chloro-N-[4-(4-pyridin-4-yl-pyrimidin-2-ylamino)-phenyl]-acetamide
Compound 126 2-Chloro-N-[4-(4-pyridin-2-yl-pyrimidin-2-ylamino)-phenyl]-acetamide
Compound 127 2-Chloro-N-[4-(4-pyridin-3-yl-pyrimidin-2-ylamino)-phenyl]-acetamide
Compound 128 2-Chloro-N-[3-(4-pyridin-3-yl-pyrimidin-2-ylamino)-phenyl]-acetamide
Compound 129 2-Chloro-N-[3-(4-pyridin-4-yl-pyrimidin-2-ylamino)-phenyl]-acetamide
Compound 130 2-Chloro-N-[3-(4-pyridin-2-yl-pyrimidin-2-ylamino)-phenyl]-acetamide
Compound 131 2-(4-Methyl-piperazin-1-yl)-N-[3-(4-pyridin-3-yl-pyrimidin-2-ylamino)-phenyl]-acetamide
Compound 132 N*3*-[4-(4-Imidazol-1-yl-phenyl)-pyrimidin-2-yl]-4-methyl-benzene-1,3-diamine
Compound 133 4-Chloro-N-{3-[4-(4-imidazol-1-yl-phenyl)-pyrimidin-2-ylamino]-4-methyl-phenyl}-benzamide
Compound 134 N-{3-[4-(4-Imidazol-1-yl-phenyl)-pyrimidin-2-ylamino]-4-methyl-phenyl}-4-methyl-benzamide
Compound 135 [4-(4-Imidazol-1-yl-phenyl)-pyrimidin-2-yl]-(3-nitro-phenyl)-amine
Compound 136 N-{3-[4-(4-Imidazol-1-yl-phenyl)-pyrimidin-2-ylamino]-4-methyl-phenyl}-3,4,5-trimethoxy-benzamide
Compound 137 4-Cyano-N-{3-[4-(4-imidazol-1-yl-phenyl)-pyrimidin-2-ylamino]-4-methyl-phenyl}-benzamide
Compound 138 N-{3-[4-(4-Imidazol-1-yl-phenyl)-pyrimidin-2-ylamino]-4-methyl-phenyl}-nicotinamide
Compound 139 Thiophene-2-sulfonic acid {3-[4-(4-imidazol-1-yl-phenyl)-pyrimidin-2-ylamino]-4-methyl-phenyl}-amide
Compound 140 Naphthalene-2-carboxylic acid {3-[4-(4-imidazol-1-yl-phenyl)-pyrimidin-2-ylamino]-4-methyl-phenyl}-amide
Compound 141 N-{3-[4-(4-Imidazol-1-yl-phenyl)-pyrimidin-2-ylamino]-4-methyl-phenyl}-2-methoxy-benzamide
Compound 142 N-[4-(4-Imidazol-1-yl-phenyl)-pyrimidin-2-yl]-benzene-1,3-diamine
Compound 143 N,N′-Bis-[4-(4-imidazol-1-yl-phenyl)-pyrimidin-2-yl]-benzene-1,3-diamine
Compound 144 [4-(3,4-Dimethoxy-phenyl)-pyrimidin-2-yl]-(2-methyl-5-nitro-phenyl)-amine
Compound 145 [4-(3,4-Dimethoxy-phenyl)-pyrimidin-2-yl]-(3-nitro-phenyl)-amine
Compound 146 4-Chloromethyl-N-{3-[4-(4-imidazol-1-yl-phenyl)-pyrimidin-2-ylamino]-4-methyl-phenyl}-benzamide
Compound 147 N-[4-(3,4-Dimethoxy-phenyl)-pyrimidin-2-yl]-benzene-1,3-diamine
Compound 148 4-Cyano-N-{3-[4-(4-imidazol-1-yl-phenyl)-pyrimidin-2-ylamino]-phenyl}-benzamide
Compound 149 Naphthalene-2-carboxylic acid {3-[4-(4-imidazol-1-yl-phenyl)-pyrimidin-2-ylamino]-phenyl}-amide
Compound 150 N-{3-[4-(4-Imidazol-1-yl-phenyl)-pyrimidin-2-ylamino]-phenyl}-nicotinamide
Compound 151 N-{3-[4-(4-Imidazol-1-yl-phenyl)-pyrimidin-2-ylamino]-phenyl}-3,4,5-trimethoxy-benzamide
Compound 152 4-Chloro-N-{3-[4-(4-imidazol-1-yl-phenyl)-pyrimidin-2-ylamino]-phenyl}-benzamide
Compound 153 Naphthalene-2-carboxylic acid {3-[4-(3,4-dimethoxy-phenyl)-pyrimidin-2-ylamino]-4-methyl-phenyl}-amide
Compound 154 N-{3-[4-(3,4-Dimethoxy-phenyl)-pyrimidin-2-ylamino]-4-methyl-phenyl}-4-methyl-benzamide
Compound 155 4-Chloromethyl-N-{3-[4-(3,4-dimethoxy-phenyl)-pyrimidin-2-ylamino]-4-methyl-phenyl}-benzamide
Compound 156 N-{3-[4-(3,4-Dimethoxy-phenyl)-pyrimidin-2-ylamino]-4-methyl-phenyl}-4-(4-methyl-piperazin-1-ylmethyl)-benzamide
Compound 157 N-{3-[4-(4-Imidazol-1-yl-phenyl)-pyrimidin-2-ylamino]-phenyl}-4-methyl-benzamide
Compound 158 N-{3-[4-(4-Imidazol-1-yl-phenyl)-pyrimidin-2-ylamino]-phenyl}-2-methoxy-benzamide
Compound 159 N-{3-[4-(3,4-Dihydroxy-phenyl)-pyrimidin-2-ylamino]-4-methyl-phenyl}-4-(4-methyl-piperazin-1-ylmethyl)-benzamide
Compound 160 4-Chloromethyl-N-{3-[4-(4-imidazol-1-yl-phenyl)-pyrimidin-2-ylamino]-phenyl}-benzamide
Compound 161 4-Chloro-N-{3-[4-(3,4-dimethoxy-phenyl)-pyrimidin-2-ylamino]-4-methyl-phenyl}-benzamide
Compound 162 N-{3-[4-(3,4-Dimethoxy-phenyl)-pyrimidin-2-ylamino]-4-methyl-phenyl}-3,4,5-trimethoxy-benzamide
Compound 163 N-3{-[4-(3,4-Dimethoxy-phenyl)-pyrimidin-2-yl]}-4-methyl-benzene-1,3-diamine
Compound 164 4-Cyano-N-{3-[4-(3,4-dimethoxy-phenyl)-pyrimidin-2-ylamino]-4-methyl-phenyl}-benzamide
Compound 165 N-{3-[4-(3,4-Dimethoxy-phenyl)-pyrimidin-2-ylamino]-4-methyl-phenyl}-nicotinamide
Compound 166 N-{3-[4-(3,4-Dimethoxy-phenyl)-pyrimidin-2-ylamino]-4-methyl-phenyl}-2-methoxy-benzamide
Compound 167 N-(3-Methyl-isoxazolo[5,4-d]pyrimidin-4-yl)-N′-(4-pyridin-3-yl-pyrimidin-2-yl)-benzene-1,3-diamine
Compound 168 N-(3-Methyl-isoxazolo[5,4-d]pyrimidin-4-yl)-N′-(4-pyridin-4-yl-pyrimidin-2-yl)-benzene-1,3-diamine
Compound 169 5-(1-Imino-ethyl)-6-[4-(4-pyridin-4-yl-pyrimidin-2-ylamino)-phenylamino]-3H-pyrimidin-4-one
Compound 170 N-(3-Methyl-isoxazolo[5,4-d]pyrimidin-4-yl)-N′-(4-pyridin-4-yl-pyrimidin-2-yl)-benzene-1,4-diamine
Compound 171 2-(4-Methyl-piperazin-1-yl)-N-[4-(4-pyridin-4-yl-pyrimidin-2-ylamino)-phenyl]-acetamide
Compound 172 4-{[4-(4-Pyridin-4-yl-pyrimidin-2-ylamino)-phenylcarbamoyl]-methyl}-piperazine-1-carboxylic acid ethyl ester
Compound 173 2-Morpholin-4-yl-N-[4-(4-pyridin-4-yl-pyrimidin-2-ylamino)-phenyl]-acetamide
Compound 174 1-{[4-(4-Pyridin-4-yl-pyrimidin-2-ylamino)-phenylcarbamoyl]-methyl}-piperidine-4-carboxylic acid ethyl ester
Compound 175 2-Chloro-N-[4-methyl-3-(4-pyridin-3-yl-pyrimidin-2-ylamino)-phenyl]-acetamide
Compound 176 4-Methyl-N-1[-(3-methyl-isoxazolo[5,4-d]pyrimidin-4-yl)]-N′-[3-(4-pyridin-3-yl-pyrimidin-2-yl)]-benzene-1,3-diamine
Compound 177 5-(1-Imino-ethyl)-6-[4-methyl-3-(4-pyridin-3-yl-pyrimidin-2-ylamino)-phenylamino]-pyrimidin-4-ol
Compound 178 5-(1-Imino-ethyl)-6-[4-methyl-3-(4-pyridin-4-yl-pyrimidin-2-ylamino)-phenylamino]-pyrimidin-4-ol
Compound 179 [1,8]Naphthyridine-2-carboxylic acid [4-methyl-3-(4-pyridin-3-yl-pyrimidin-2-ylamino)-phenyl]-amide
Compound 180 2-Chloro-N-[4-methyl-3-(4-pyridin-2-yl-pyrimidin-2-ylamino)-phenyl]-acetamide
Compound 181 5-(1-Imino-ethyl)-6-[3-(4-pyridin-3-yl-pyrimidin-2-ylamino)-phenylamino]-pyrimidin-4-ol
Compound 182 4-(4-Methyl-piperazin-1-ylmethyl)-N-[4-(4-pyridin-2-yl-pyrimidin-2-ylamino)-phenyl]-benzamide
Compound 183 4-{[3-(4-Pyridin-3-yl-pyrimidin-2-ylamino)-phenylcarbamoyl]-methyl}-piperazine-1-carboxylic acid ethyl ester
Compound 184 2-Morpholin-4-yl-N-[3-(4-pyridin-3-yl-pyrimidin-2-ylamino)-phenyl]-acetamide
Compound 185 1-{[3-(4-Pyridin-3-yl-pyrimidin-2-ylamino)-phenylcarbamoyl]-methyl}-piperidine-4-carboxylic acid ethyl ester
Compound 186 2-(4-Methyl-piperazin-1-yl)-N-[4-(4-pyridin-2-yl-pyrimidin-2-ylamino)-phenyl]-acetamide
Compound 187 2-Morpholin-4-yl-N-[4-(4-pyridin-2-yl-pyrimidin-2-ylamino)-phenyl]-acetamide
Compound 188 4-{[4-(4-Pyridin-2-yl-pyrimidin-2-ylamino)-phenylcarbamoyl]-methyl}-piperazine-1-carboxylic acid ethyl ester
Compound 189 1-{[4-(4-Pyridin-2-yl-pyrimidin-2-ylamino)-phenylcarbamoyl]-methyl}-piperidine-4-carboxylic acid ethyl ester
Compound 190 2-(4-Methyl-piperazin-1-yl)-N-[4-methyl-3-(4-pyridin-3-yl-pyrimidin-2-ylamino)-phenyl]-acetamide
Compound 191 1-{[4-Methyl-3-(4-pyridin-3-yl-pyrimidin-2-ylamino)-phenylcarbamoyl]-methyl}-piperidine-4-carboxylic acid ethyl ester
Compound 192 4-{[4-Methyl-3-(4-pyridin-3-yl-pyrimidin-2-ylamino)-phenylcarbamoyl]-methyl}-piperazine-1-carboxylic acid ethyl ester
Compound 193 N-[4-Methyl-3-(4-pyridin-3-yl-pyrimidin-2-ylamino)-phenyl]-2-morpholin-4-yl-acetamide
Compound 194 4-Methyl-N-1[-(3-methyl-isoxazolo[5,4-d]pyrimidin-4-yl)]-N′-[3-(4-pyridin-4-yl-pyrimidin-2-yl)]-benzene-1,3-diamine
Compound 195 2-(4-Methyl-piperazin-1-yl)-N-[4-methyl-3-(4-pyridin-4-yl-pyrimidin-2-ylamino)-phenyl]-acetamide
Compound 196 N-[4-Methyl-3-(4-pyridin-4-yl-pyrimidin-2-ylamino)-phenyl]-2-morpholin-4-yl-acetamide
Compound 197 3,4,5-Trimethoxy-N-[4-(4-pyridin-4-yl-pyrimidin-2-ylamino)-phenyl]-benzamide
Compound 198 2-Methoxy-N-[4-(4-pyridin-4-yl-pyrimidin-2-ylamino)-phenyl]-benzamide
Compound 199 N-[4-(4-Pyridin-4-yl-pyrimidin-2-ylamino)-phenyl]-nicotinamide
Compound 200 Naphthalene-2-carboxylic acid [4-(4-pyridin-4-yl-pyrimidin-2-ylamino)-phenyl]-amide
Compound 201 4-Chloro-N-[4-(4-pyridin-4-yl-pyrimidin-2-ylamino)-phenyl]-benzamide
Compound 202 1-{[4-Methyl-3-(4-pyridin-4-yl-pyrimidin-2-ylamino)-phenylcarbamoyl]-methyl}-piperidine-4-carboxylic acid ethyl ester
Compound 203 Thiophene-2-sulfonic acid [4-(4-pyridin-4-yl-pyrimidin-2-ylamino)-phenyl]-amide
Compound 204 4-{[4-Methyl-3-(4-pyridin-4-yl-pyrimidin-2-ylamino)-phenyl-carbamoyl]-methyl}-piperazine-1-carboxylic acid ethyl ester
Compound 205 4-Bromo-N-[4-(4-pyridin-4-yl-pyrimidin-2-ylamino)-phenyl]-benzamide
Compound 206 2,3,4,5,6-Pentafluoro-N-[4-(4-pyridin-4-yl-pyrimidin-2-ylamino)-phenyl]-benzamide
Compound 207 2-(4-Methyl-piperazin-1-yl)-N-[4-methyl-3-(4-pyridin-2-yl-pyrimidin-2-ylamino)-phenyl]-acetamide
Compound 208 N-[4-Methyl-3-(4-pyridin-2-yl-pyrimidin-2-ylamino)-phenyl]-2-morpholin-4-yl-acetamide
Compound 209 4-Chloro-N-[4-(4-pyridin-4-yl-pyrimidin-2-ylamino)-phenyl]-benzenesulfonamide
Compound 210 Naphthalene-2-sulfonic acid [4-(4-pyridin-4-yl-pyrimidin-2-ylamino)-phenyl]-amide
Compound 211 4-Methyl-N-[3-(4-pyridin-3-yl-pyrimidin-2-ylamino)-phenyl]-benzenesulfonamide
Compound 212 [1,8]Naphthyridine-2-carboxylic acid [4-methyl-3-(4-pyridin-4-yl-pyrimidin-2-ylamino)-phenyl]-amide
Compound 213 4-(4-Methyl-piperazin-1-ylmethyl)-N-[4-methyl-3-(4-pyridin-4-yl-pyrimidin-2-ylamino)-phenyl]-benzamide
Compound 214 1-{[4-Methyl-3-(4-pyridin-2-yl-pyrimidin-2-ylamino)-phenylcarbamoyl]-methyl}-piperidine-4-carboxylic acid ethyl ester
Compound 215 4-{[4-Methyl-3-(4-pyridin-2-yl-pyrimidin-2-ylamino)-phenyl-carbamoyl]-methyl}-piperazine-1-carboxylic acid ethyl ester
Compound 216 4-Chloro-N-[4-(4-pyridin-2-yl-pyrimidin-2-ylamino)-phenyl]-benzenesulfonamide
Compound 217 Naphthalene-2-sulfonic acid [4-(4-pyridin-2-yl-pyrimidin-2-ylamino)-phenyl]-amide
Compound 218 4-Chloro-N-[4-(4-pyridin-3-yl-pyrimidin-2-ylamino)-phenyl]-benzenesulfonamide
Compound 219 Naphthalene-2-sulfonic acid [4-(4-pyridin-3-yl-pyrimidin-2-ylamino)-phenyl]-amide
Compound 220 2-Methoxy-N-[4-methyl-3-(4-pyridin-4-yl-pyrimidin-2-ylamino)-phenyl]-benzamide
Compound 221 N-(3-Methyl-isoxazolo[5,4-d]pyrimidin-4-yl)-N′-(4-pyridin-2-yl-pyrimidin-2-yl)-benzene-1,4-diamine
Compound 222 Naphthalene-2-carboxylic acid [4-methyl-3-(4-pyridin-4-yl-pyrimidin-2-ylamino)-phenyl]-amide
Compound 223 N-[4-Methyl-3-(4-pyridin-4-yl-pyrimidin-2-ylamino)-phenyl]-benzamide
Compound 224 2-(4-Methyl-piperazin-1-yl)-N-[3-(4-pyridin-4-yl-pyrimidin-2-ylamino)-phenyl]-acetamide
Compound 225 4-Methyl-N-[4-methyl-3-(4-pyridin-4-yl-pyrimidin-2-ylamino)-phenyl]-benzamide
Compound 226 2-Morpholin-4-yl-N-[3-(4-pyridin-4-yl-pyrimidin-2-ylamino)-phenyl]-acetamide
Compound 227 Naphthalene-2-sulfonic acid [4-methyl-3-(4-pyridin-4-yl-pyrimidin-2-ylamino)-phenyl]-amide
Compound 228 1-{[3-(4-Pyridin-4-yl-pyrimidin-2-ylamino)-phenylcarbamoyl]-methyl}-piperidine-4-carboxylic acid ethyl ester
Compound 229 4-{[3-(4-Pyridin-4-yl-pyrimidin-2-ylamino)-phenylcarbamoyl]-methyl}-piperazine-1-carboxylic acid ethyl ester
Compound 230 1-{[3-(4-Pyridin-4-yl-pyrimidin-2-ylamino)-phenylcarbamoyl]-methyl}-piperidine-4-carboxylic acid
Compound 231 Cyclohexanecarboxylic acid [4-methyl-3-(4-pyridin-4-yl-pyrimidin-2-ylamino)-phenyl]-amide
Compound 232 N,N′-Bis-[4-(3,4-dimethoxy-phenyl)-pyrimidin-2-yl]-benzene-1,3-diamine
Compound 233 N-[4-(4-Imidazol-1-yl-phenyl)-pyrimidin-2-yl]-N′-(3-methyl-isoxazolo[5,4-d]pyrimidin-4-yl)-benzene-1,3-diamine
Compound 234 3-Fluoro-N-{3-[4-(4-imidazol-1-yl-phenyl)-pyrimidin-2-ylamino]-phenyl}-benzamide
Compound 235 N-{3-[4-(3,4-Dimethoxy-phenyl)-pyrimidin-2-ylamino]-4-methyl-phenyl}-3-fluoro-benzamide
Compound 236 N-{3-[4-(4-Imidazol-1-yl-phenyl)-pyrimidin-2-ylamino]-phenyl}-4-(4-methyl-piperazin-1-ylmethyl)-benzamide
Compound 237 N-{3-[4-(3,4-Dimethoxy-phenyl)-pyrimidin-2-yl]}-4-methyl-N′-[1-(3-methyl-isoxazolo[5,4-d]pyrimidin-4-yl)]-benzene-1,3-diamine
Compound 238 3-Fluoro-N-{3-[4-(4-imidazol-1-yl-phenyl)-pyrimidin-2-ylamino]-4-methyl-phenyl}-benzamide
Compound 239 N-{3-[4-(3,4-Dimethoxy-phenyl)-pyrimidin-2-ylamino]-phenyl}-3-fluoro-benzamide
Compound 240 N-{3-[4-(4-Imidazol-1-yl-phenyl)-pyrimidin-2-yl]}-4-methyl-N′-[1-(3-methyl-isoxazolo[5,4-d]pyrimidin-4-yl)]-benzene-1,3-diamine
Compound 241 4-Chloro-N-{3-[4-(3,4-dimethoxy-phenyl)-pyrimidin-2-ylamino]-phenyl}-benzamide
Compound 242 N-{3-[4-(3,4-Dimethoxy-phenyl)-pyrimidin-2-ylamino]-phenyl}-4-methyl-benzamide
Compound 243 N-{3-[4-(3,4-Dimethoxy-phenyl)-pyrimidin-2-ylamino]-phenyl}-3,4,5-trimethoxy-benzamide
Compound 244 4-Cyano-N-{3-[4-(3,4-dimethoxy-phenyl)-pyrimidin-2-ylamino]-phenyl}-benzamide
Compound 245 4-Chloromethyl-N-{3-[4-(3,4-dimethoxy-phenyl)-pyrimidin-2-ylamino]-phenyl}-benzamide
Compound 246 Naphthalene-2-carboxylic acid {3-[4-(3,4-dimethoxy-phenyl)-pyrimidin-2-ylamino]-phenyl}-amide
Compound 247 N-{3-[4-(3,4-Dimethoxy-phenyl)-pyrimidin-2-ylamino]-phenyl}-2-methoxy-benzamide
Compound 248 N-{3-[4-(3,4-Dimethoxy-phenyl)-pyrimidin-2-ylamino]-phenyl}-nicotinamide
Compound 249 N-{3-[4-(3,4-Dimethoxy-phenyl)-pyrimidin-2-ylamino]-phenyl}-4-(4-methyl-piperazin-1-ylmethyl)-benzamide
Compound 250 N-[4-(3,4-Dimethoxy-phenyl)-pyrimidin-2-yl]-N′-(3-methyl-isoxazolo[5,4-d]pyrimidin-4-yl)-benzene-1,3-diamine
Compound 251 Isoquinoline-5-sulfonic acid {3-[4-(3,4-dimethoxy-phenyl)-pyrimidin-2-ylamino]-4-methyl-phenyl}-amide
Compound 252 N-{3-[4-(4-Imidazol-1-yl-phenyl)-pyrimidin-2-ylamino]-4-methyl-phenyl}-4-(4-methyl-piperazin-1-ylmethyl)-benzamide
Compound 253 (4-Benzo[1,3]dioxol-5-yl-pyrimidin-2-yl)-(2-methyl-5-nitro-phenyl)-amine
Compound 254 4-(4-Methyl-piperazin-1-ylmethyl)-N-[4-(4-pyridin-4-yl-pyrimidin-2-ylamino)-phenyl]-benzamide
Compound 255 N-[4-(4-Imidazol-1-yl-phenyl)-pyrimidin-2-yl]-benzene-1,4-diamine
Compound 256 N-[4-(3,4-Dimethoxy-phenyl)-pyrimidin-2-yl]-benzene-1,4-diamine
Compound 257 Naphthalene-2-carboxylic acid {4-[4-(4-imidazol-1-yl-phenyl)-pyrimidin-2-ylamino]-phenyl}-amide
Compound 258 (3-Chloro-phenyl)-(4-pyridin-4-yl-pyrimidin-2-yl)-amine
Compound 259 [1,8]Naphthyridine-2-carbothioic acid [4-methyl-3-(4-pyridin-4-yl-pyrimidin-2-ylamino)-phenyl]-amide
Compound 260 4-Methyl-N-[4-methyl-3-(4-pyridin-3-yl-pyrimidin-2-ylamino)-phenyl]-benzenesulfonamide
Compound 261 3-Chloro-N-[4-methyl-3-(4-pyridin-4-yl-pyrimidin-2-ylamino)-phenyl]-propionamide
Compound 262 3-(4-Methyl-piperazin-1-yl)-N-[4-methyl-3-(4-pyridin-4-yl-pyrimidin-2-ylamino)-phenyl]-propionamide
Compound 263 4-{2-[4-Methyl-3-(4-pyridin-4-yl-pyrimidin-2-ylamino)-phenylcarbamoyl]-ethyl}-piperazine-1-carboxylic acid ethyl ester
Compound 264 2-(4-Methyl-piperazin-1-yl)-N-[3-(4-pyridin-2-yl-pyrimidin-2-ylamino)-phenyl]-acetamide
Compound 265 2-Morpholin-4-yl-N-[3-(4-pyridin-2-yl-pyrimidin-2-ylamino)-phenyl]-acetamide
Compound 266 N-[4-Methyl-3-(4-pyridin-2-yl-pyrimidin-2-ylamino)-phenyl]-nicotinamide
Compound 267 1-{[3-(4-Pyridin-2-yl-pyrimidin-2-ylamino)-phenylcarbamoyl]-methyl}-piperidine-4-carboxylic acid ethyl ester
Compound 268 4-{[3-(4-Pyridin-2-yl-pyrimidin-2-ylamino)-phenylcarbamoyl]-methyl}-piperazine-1-carboxylic acid ethyl ester
Compound 269 Naphthalene-2-carboxylic acid [4-methyl-3-(4-pyridin-2-yl-pyrimidin-2-ylamino)-phenyl]-amide
Compound 270 4-Bromo-N-[4-methyl-3-(4-pyridin-2-yl-pyrimidin-2-ylamino)-phenyl]-benzamide
Compound 271 1-{[3-(4-Pyridin-2-yl-pyrimidin-2-ylamino)-phenylcarbamoyl]-methyl}-piperidine-4-carboxylic acid
Compound 272 4-Methyl-N-[4-methyl-3-(4-pyridin-2-yl-pyrimidin-2-ylamino)-phenyl]-benzamide
Compound 273 Naphthalene-2-sulfonic acid [4-methyl-3-(4-pyridin-2-yl-pyrimidin-2-ylamino)-phenyl]-amide
Compound 274 4-Chloromethyl-N-[4-methyl-3-(4-pyridin-2-yl-pyrimidin-2-ylamino)-phenyl]-benzamide
Compound 275 2-Methoxy-N-[4-methyl-3-(4-pyridin-2-yl-pyrimidin-2-ylamino)-phenyl]-benzamide
Compound 276 4-(4-Methyl-piperazin-1-ylmethyl)-N-[4-methyl-3-(4-pyridin-2-yl-pyrimidin-2-ylamino)-phenyl]-benzamide
Compound 277 4-Fluoro-N-[4-methyl-3-(4-pyridin-2-yl-pyrimidin-2-ylamino)-phenyl]-benzenesulfonamide
Compound 278 Cyclopentanecarboxylic acid [4-methyl-3-(4-pyridin-2-yl-pyrimidin-2-ylamino)-phenyl]-amide
Compound 279 3-{4-[2-(3-Chloro-phenylamino)-pyrimidin-4-yl]-pyridin-2-ylamino}-propan-1-ol
Compound 280 Isoquinoline-5-sulfonic acid {3-[4-(4-imidazol-1-yl-phenyl)-pyrimidin-2-ylamino]-4-methyl-phenyl}-amide
Compound 281 (2-Methyl-5-nitro-phenyl)-(4-pyridin-3-yl-pyrimidin-2-yl)-amine
Compound 282 (3-Chloro-phenyl)-[4-(2-chloro-pyridin-4-yl)-pyrimidin-2-yl]-amine
Compound 283 4-Chloromethyl-N-[3-(4-pyridin-4-yl-pyrimidin-2-ylamino)-phenyl]-benzamide
Compound 284 Thiophene-2-sulfonic acid {3-[4-(3,4-dimethoxy-phenyl)-pyrimidin-2-ylamino]-4-methyl-phenyl}-amide
Compound 285 4-(4-Methyl-piperazin-1-ylmethyl)-N-[3-(4-pyridin-4-yl-pyrimidin-2-ylamino)-phenyl]-benzamide
Compound 286 N-{1-{5-[2-(3,4,5-Trimethoxy-phenylamino)-pyrimidin-4-yl]-pyridin-2-yl}}-ethane-1,2-diamine
Compound 287 (4-(6-Dimethylamino-pyridin-3-yl)-pyrimidin-2-yl]-(3,4,5-trimethoxy-phenyl)-amine
Compound 288 2,2-Dimethyl-N-{3-[2-(2-methyl-5-nitro-phenylamino)-pyrimidin-4-yl]-pyridin-4-yl}-propionamide
Compound 289 [4-(4-Amino-pyridin-3-yl)-pyrimidin-2-yl]-(2-methyl-5-nitro-phenyl)-amine
Compound 290 3-{5-[2-(3-Chloro-phenylamino)-pyrimidin-4-yl]-pyridin-2-ylamino}-propan-1-ol
Compound 291 (3-Chloro-phenyl)-[4-(6-chloro-pyridin-3-yl)-pyrimidin-2-yl]-amine
Compound 292 N-{1-{4-[2-(3-Trifluoromethyl-phenylamino)-pyrimidin-4-yl]-pyridin-2-yl}}-ethane-1,2-diamine
Compound 293 (4-Pyridin-4-yl-pyrimidin-2-yl)-(3-trifluoromethyl-phenyl)-amine
Compound 294 [4-(1-Oxy-pyridin-4-yl)-pyrimidin-2-yl]-(3-trifluoromethyl-phenyl)-amine
Compound 295 3-(4-Pyridin-4-yl-pyrimidin-2-ylamino)-benzoic acid ethyl ester
Compound 296 3-[4-(1-Oxy-pyridin-4-yl)-pyrimidin-2-ylamino]-benzoic acid ethyl ester
Compound 297 3-{4-[2-(3-Trifluoromethyl-phenylamino)-pyrimidin-4-yl]-pyridin-2-ylamino}-propan-1-ol
Compound 298 2-{4-[2-(3-Trifluoromethyl-phenylamino)-pyrimidin-4-yl]-pyridin-2-ylamino}-ethanol
Compound 299 5-{4-[2-(3-Trifluoromethyl-phenylamino)-pyrimidin-4-yl]-pyridin-2-ylamino}-pentan-1-ol
Compound 300 {4-[2-(3-Imidazol-1-yl-propylamino)-pyridin-4-yl]-pyrimidin-2-yl}-(3-trifluoromethyl-phenyl)-amine
Compound 301 (4-{2-[3-(4-Methyl-piperazin-1-yl)-propylamino]-pyridin-4-yl}-pyrimidin-2-yl)-(3-trifluoromethyl-phenyl)-amine
Compound 302 3-(4-Pyridin-4-yl-pyrimidin-2-ylamino)-benzoic acid
Compound 303 N-(3-Hydroxy-propyl)-3-{4-[2-(3-hydroxy-propylamino)-pyridin-4-yl]-pyrimidin-2-ylamino}-benzamide
Compound 304 3-[4-(2-Chloro-pyridin-4-yl)-pyrimidin-2-ylamino]-benzoic acid
Compound 305 3-{4-[2-(3-Hydroxy-propylamino)-pyridin-4-yl]-pyrimidin-2-ylamino}-benzoic acid
Compound 306 1-[3-(4-Pyridin-4-yl-pyrimidin-2-ylamino)-benzoyl]-piperidine-4-carboxylic acid ethyl ester
Compound 307 3-{4-[2-(3-Hydroxy-propylamino)-pyridin-4-yl]-pyrimidin-2-ylamino}-benzoic acid methyl ester
Compound 308 N-(4,4-Diethoxy-butyl)-3-(4-pyridin-4-yl-pyrimidin-2-ylamino)-benzamide
Compound 309 [4-(2-Chloro-pyridin-4-yl)-pyrimidin-2-yl]-(3-methylsulfanyl-phenyl)-amine
Compound 310 2-{4-[2-(3-Methylsulfanyl-phenylamino)-pyrimidin-4-yl]-pyridin-2-ylamino}-ethanol
Compound 311 5-{4-[2-(3-Methylsulfanyl-phenylamino)-pyrimidin-4-yl]-pyridin-2-ylamino}-pentan-1-ol
Compound 312 3-{4-[2-(3-Methylsulfanyl-phenylamino)-pyrimidin-4-yl]-pyridin-2-ylamino}-propan-1-ol
Compound 313 [4-(2-Chloro-pyridin-4-yl)-pyrimidin-2-yl]-(3-trifluoromethyl-phenyl)-amine.

The afore-mentioned specific compounds as well as all compounds falling within the ambit of any one of the general formulas (I), (Ia)-(Ix) and/or pharmaceutically acceptable salts thereof, can be used as pharmaceutically active agents. Consequently, one aspect of the present invention is directed to the compounds of general formula (I) or (Ia)-(Ix) and or pharmaceutically acceptable salts thereof for use as pharmaceutically active agent. Furthermore, it was found that the inventive compounds are inhibitors of kinases and phosphatases, especially human protein kinases.

Other aspects of the present invention relate to the pyrimidine derivatives of the general formula (I) or any one of the general formulas (Ia)-(Ix) as new pharmaceutically active agents, especially for the preparation of a pharmaceutical composition for the treatment of diseases and disorders which are cured or relieved or which can be cured or relieved by the inhibition of a kinase and/or phosphatase. The compounds of the general formulas (I), (Ia)-(Ix) were surprisingly identified as potent inhibitors for especially human but also viral kinases. Such target kinases are for example Abl, Akt, c-kit, EGF-R, GSK3b, JNK, Lck, PDGF-R, PknG, and ROCK2.

As used herein, the term “inhibitor” refers to any compound capable of downregulating, decreasing, suppressing or otherwise regulating the amount and/or activity of the human cellular protein kinase Abl, Akt, c-kit, EGF-R, GSK3b, JNK, Lck, PDGF-R, PknG, or ROCK2.

Thus, a method is disclosed herein for preventing and/or treating diseases which are cured or relieved by the inhibition of kinases and/or phosphatases in a mammal, including a human, which method comprises administering to the mammal an amount of at least one compound of general formula (I), (Ia)-(Ix) and/or pharmaceutically acceptable salts thereof, effective to prevent and/or treat said diseases which are cured or relieved by the inhibition of a kinase and/or phosphatase. A preferred embodiment of said method involves one of the following kinases: Abl, Akt, c-kit, EGF-R, GSK3b, JNK, Lck, PDGF-R, PknG, or ROCK2.

The nucleoside sequences of the genes coding for the human cellular protein kinases Abl, Akt, c-kit, EGF-R, GSK3b, JNK, Lck, PDGF-R, PknG, and ROCK2 as well as their amino acid sequences can be obtained from NCBI (National Library of Medicine: PubMed), SwissPort, GenBank, or EMBL. The accession numbers for said kinases are:

AbI (Accession Number: M14752), Akt1 (Accession Number: M63167), c-kit (Accession Number: GenBank X06182), EGF-R (Accession Number: AF288738), GSK3β (Accession Number: SwissProt P49841), JNK (Accession Number: Jnk1a1: EMBL L26318), Lck (Accession Number: SwissProt P06239), PDGF-Rβ (Accession Number: GenBank J03278), PknG (Accession Number: NC000962, not the complete genome), and ROCK2 (Accession Number: NM004850).

### Cancer

As revealed for the first time herein, the present invention discloses the use of compounds of any one of the general formulas (I), (Ia)-(Ix) and/or pharmaceutically acceptable salts thereof for prophylaxis and/or treatment of proliferation disorder and cancer.

The proliferation disorders and cancers are preferably selected from the group comprising adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gallbladder cancer, gall bladder carcinomas, uterine cancer, cervical cancer, glioblastomas, gynecologic tumors, ear, nose and throat tumors, hematologic neoplasias, hairy cell leukemia, urethral cancer, skin cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

### Cardiovascular Diseases and Disorders

Another aspect of the present invention is directed to the use of at least one compound of any one of the general formulas (I), (Ia)-(Ix) and/or pharmaceutically acceptable salts thereof for prophylaxis and/or treatment of cardiovascular diseases and cardiovascular disorders.

Examples of cardiovascular diseases and disorders are: aneurysm, stable angina, unstable angina, angina pectoris, angioneurotic edema, stenosis, restenosis, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, Sneddon syndrome, superior vena cava syndrome, syndrome X, tachycardia, Takayasu's arteritis, hereditary hemorrhagic telangiectasia, telangiectasis, temporal arteritis, tetralogy of fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

### Inflammation

Another aspect of the present invention is directed to the use of at least one compound of any one of the general formulas (I), (Ia)-(Ix) and/or pharmaceutically acceptable salts thereof for prophylaxis and/or treatment of inflammatory diseases.

The inventive compounds according to any one of the formulas (I), (Ia)-(Ix) are useful for prophylaxis and/or treatment of diseases which are associated with overexpression/overproduction of the protein amyloid A, such as arthritides, rheumatoid arthritis, asthma, lupus, bleeding disorders (thrombocytopenia), chronic inflammatory lung diseases, atherosclerosis, kidney inflammation (nephritis), psoriasis, allergies, Crohn's disease, ischemia/reperfusion injury, endotoxemic liver injury, inflammatory bowel disease, tuberculosis, chronic infections, familial Mediterranean fever, interstitial cystitis and skin sunburn.

### Neurodegenerative Disorders

Another aspect of the present invention is directed to the use of at least one compound of any one of the general formulas (I), (Ia)-(Ix) and/or pharmaceutically acceptable salts thereof for prophylaxis and/or treatment of neuro-degeneration and neurodegenerative disorders.

Among the hundreds of different neurodegenerative disorders, the attention has been given only to a handful, including Alzheimer disease, Parkinson disease, Huntington disease, and amyotrophic lateral sclerosis.

It is worth to mention that the same neurodegenerative process can affect different areas of the brain, making a given disease appear very different from a symptomatic stand-point.

Neurodegenerative disorders of the central nervous system (CNS) can be grouped into diseases of the cerebral cortex (Alzheimer disease), the basal ganglia (Parkinson disease), the brain-stem and cerebellum, or the spinal cord (amyotrophic lateral sclerosis).

Examples for neurodegeneration and neurodegenerative disorders are Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA).

### Immunological Diseases

Another aspect of the present invention is directed to the use of at least one compound of any one of the general formulas (I), (Ia)-(Ix) and/or pharmaceutically acceptable salts thereof for prophylaxis and/or treatment of immunological diseases, neuroimmunological diseases, autoimmune diseases.

Immunological diseases are, for instance, asthma and diabetes, rheumatic and autoimmune diseases, AIDS, rejection of transplanted organs and tissues (cf. below), rhinitis, chronic obstructive pulmonary diseases, osteoporisis, ulcerative colitis, sinusitis, lupus erythematosus, recurrent infections, atopic dermatitis/eczema and occupational allergies, food allergies, drug allergies, severe anaphylactic reactions, anaphylaxis, and other manifestations of allergic disease, as well as uncommon problems such as primary immunodeficiencies, including antibody deficiency states, cell mediated immunodeficiencies (e.g., severe combined immunodeficiency, DiGeorge syndrome, Hyper-IgE syndrome, Wiskott-Aldrich syndrome, ataxia-telangiectasia), immune mediated cancers, and white cell defects.

In autoimmune diseases, such as systemic lupus erythematosus, rheumatoid arthritis (RA), multiple sclerosis (MS), immune-mediated or type 1 diabetes mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, and Hashimoto's disease, dermatomyositis, goodpastture syndrome, myasthenia gravis pseudoparalytica, ophtalmia sympatica, phakogene uveitis, chronical agressivce hepatitis, primary billiary cirrhosis, autoimunehemolytic anemy, Werlof disease, specific cells uncontrollably attack the body's own tissues and organs (autoimmunity), producing inflammatory reactions and other serious symptoms and diseases.

Hashimoto's thyroiditis is one of the most common autoimmune diseases. “Autoimmune disease” refers to a category of more than 80 chronic illnesses, each very different in nature, that can affect everything from the endocrine glands (like the thyroid) to organs like the kidneys, as well as to the digestive system.

There are many different autoimmune diseases, and they can each affect the body in different ways. For example, the autoimmune reaction is directed against the brain in multiple sclerosis and the gut in Crohn's disease. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus.

### Infective Diseases

Another aspect of the present invention is directed to the use of at least one compound of any one of the general formulas (I), (Ia)-(Ix) and/or pharmaceutically acceptable salts thereof for prophylaxis and/or treatment of infective diseases including-opportunistic infections.

Examples of infective diseases are AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever.

### Transplant Rejection

Another aspect of the present invention is directed to the use of at least one compound of any one of the general formulas (I), (Ia)-(Ix) and/or pharmaceutically acceptable salts thereof for prophylaxis and/or treatment of transplant rejection.

Transplant rejection is when a transplant recipient's immune system attacks a transplanted organ or tissue. No two people (except identical twins) have identical tissue antigens. Therefore, in the absence of immunosuppressive drugs, organ and tissue transplantation would almost always cause an immune response against the foreign tissue (rejection), which would result in destruction of the transplant. Though tissue typing ensures that the organ or tissue is as similar as possible to the tissues of the recipient, unless the donor is an identical twin, no match is perfect and the possibility of organ/tissue rejection remains.

The inventive compounds of any one of the general formulas (I), (Ia)-(Ix) are used as immunosuppressive drugs and/or anti-rejection drugs in order to prevent transplant rejection.

One example of transplant rejection is the graft-versus-host-disease (GVHD) that can occur following bone marrow transplant. The donor's immune cells in the transplanted marrow make antibodies against the host's (transplant patient's) tissues and attack the patient's vital organs. Transplant rejections (also known as graft rejection or tissue/organ rejection) may commonly occur when tissue or organs which need blood supply are transplanted. Said organs comprise especially inner organs such as heart, heart-lungs, lungs, liver, kidney, pancreas, spleen, skin, tissue, bone marrow, spinal marrow, hormone producing glands, gonads and gonadal glands.

### Prion Diseases

Another aspect of the present invention is directed to the use of at least one compound of any one of the general formulas (I), (Ia)-(Ix) and/or pharmaceutically acceptable salts thereof for prophylaxis and/or treatment of prion diseases.

Prions are infectious agents which do not have a nucleic acid genome. It seems that a protein alone is the infectious agent. A prion has been defined as “small proteinaceous infectious particle which resists inactivation by procedures that modify nucleic acids”. The discovery that proteins alone can transmit an infectious disease has come as a considerable surprise to the scientific community. Prion diseases are often called “transmissible spongiform encephalopathies”, because of the post mortem appearance of the brain with large vacuoles in the cortex and cerebellum. Probably most mammalian species develop these diseases. Prion diseases are a group of neurodegenerative disorders of humans and animals and the prion diseases can manifest as sporadic, genetic or infectious disorders. Examples for prion diseases acquired by exogenous infection are the Bovine spongiform encephalitis (BSE) of cattle and the new variant of Creutzfeld-Jakob disease (vCJD) caused by BSE as well as scrapie of animals. Examples of human prion diseases include kuru, sporadic Creutzfeldt-Jakob disease (sCJD), familial CJD (fCJD), iatrogenic CJD (iCJD), Gerstmann-Sträussler-Scheinker (GSS) disease, fatal familial insomnia (FFI), and especially the new variant CJD (nvCJD or vCJD).

The name “prion” is used to describe the causative agents which underlie the transmissible spongiform encephalopathies. A prion is proposed to be a novel infectious particle that differs from viruses and viroids. It is composed solely of one unique protein that resists most inactivation procedures such as heat, radiation, and proteases. The latter characteristic has led to the term protease-resistant isoform of the prion protein. The protease-resistant isoform has been proposed to slowly catalyze the conversion of the normal prion protein into the abnormal form.

The term “isoform” in the context of prions means two proteins with exactly the same amino acid sequence that are folded into molecules with dramatically different tertiary structures. The normal cellular isoform of the prion protein (PrP^{C}) has a high α-helix content, a low β-sheet content, and is sensitive to protease digestion. The abnormal, disease-causing isoform (PrP^{Sc}) has a lower α-helix content, a much higher β-sheet content, and is much more resistant to protease digestion.

As used herein the term “prion diseases” refers to transmissible spongiform encephalopathies. Examples for prion diseases comprise Scrapie (sheep, goat), TME (transmissible mink encephalopathy; mink), CWD (chronic wasting disease; muledeer, deer, elk), BSE (bovine spongiform encephalopathy; cows, cattles), CJD (Creutzfeld-Jacob Disease), vCJD, GSS (Gerstmann-Sträussler-Scheinker syndrome), FFI (Fatal familial Insomnia), Kuru, and Alpers Syndrome. Preferred are BSE, vCJD, and CJD.

A further aspect of the present invention relates to a method for detecting prion infections and/or prion diseases in a sample comprising:

a) providing a sample from an individual; and
b) adding to said sample at least one compound of the general formula (I), (Ia)-(Ix) and/or pharmaceutically active salts thereof; and
c) detecting activity in said sample of the human cellular protein kinase Abl.

As used herein the term “sample” refers to any sample that can be taken from a living animal or human for diagnostic purposes, especially said sample comprises blood, milk, saliva, sputum, excrement, urine, spinal cord liquid, liquor, cerebral extract, lachrymal gland liquid, and biopsies.

The term “individual” preferably refers to mammals, especially humans or ruminants.

As used herein the term “ruminants” refers to an animal, for instance, cattle, cow, sheep, goat, deer, elk, muledeer, or buffalo that has four separate stomach chambers, and is therefore able to digest a wide range of organic and plant foods. The term “ruminants” refers also to exotic ruminants, like captive nyala, gemsbok, Arabian oryx, eland, kudu, scimitar-horned oryx, ankole, or bison which are also accessible to develop Spongiform encephalopathy. Minks are an example for mammals which do not belong to the species of ruminants.

Still a further aspect of the present invention is directed to pharmaceutical compositions comprising at least one pyrimidine compound of the general formula (I), (Ia)-(Ix) as an active ingredient together with at least one pharmaceutically acceptable carrier, excipient or diluents. Said pharmaceutical compositions may be formulated as pills, tablets, tabs, film tablets, coated tablets (dragees), multi-layer tablets, capsules, powders, granulates, deposits, sustained release formulations, controlled release formulations, mini- and micro-formulations, nano-formulations, liposomal formulations, dispersions, suspensions, liquid formulations, drops, injections, sprays, ointments, creams, pastes, syrup, lotions, gels, chayavanprashes.

The compounds of the general formula (I), (Ia)-(Ix) can also be administered in form of their pharmaceutically active salts optionally using substantially nontoxic pharmaceutically acceptable carriers, excipients or diluents. The medications of the present invention are prepared in a conventional solid or liquid carrier or diluents and a conventional pharmaceutically-made adjuvant at suitable dosage level in a known way. The preferred preparations are in administratable form which is suitable for oral application. These administratable forms, for example, include pills, tablets, film tablets, coated tablets, capsules, powders and deposits as well as mini- or micro formulations.

Thus, the subject of the present invention also includes pharmaceutical preparations for parenteral, including dermal, intradermal, intragastrical, intracutaneous, intravasal, intravenous, intramuscular, intraperitoneal, intranasal, intravaginal, intrabuccal, percutaneous, intraocular, rectal, subcutaneous, sublingual, topical or transdermal application, which in addition to typical vehicles and diluents contain at least one pyrimidine compound of the general formula (I), (Ia)-(Ix) and/or a pharmaceutically acceptable salt thereof as active ingredient.

The pharmaceutical compositions of the present invention, containing pyrimidine derivatives of any one of the general formulas (I), (Ia)-(Ix), will typically be administered in admixture with suitable carrier materials selected with respect to the intended form of administration, i.e. oral tablets, capsules (either solid-filled, semi-solid filled or liquid filled), powders for constitution, oral gels, elixirs, dispersible granules, syrups, suspensions, and the like, and are consistent with conventional pharmaceutical practices. For example, for oral administration in the form of tablets or capsules, the active drug component may be combined with any oral nontoxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition.

Suitable binders include starch, gelatin, natural sugars, corn sweeteners, natural and synthetic gums such as acacia, sodium alginate, carboxymethylcellulose, polyethylene glycol and waxes. Among the lubricants, there may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. antihistaminic activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidifies.

Also included are solid form preparations which are intended to be converted, shortly before use, to liquid form preparations for either oral or parenteral administration. Such liquid forms include solutions, suspensions and emulsions.

The inventive pyrimidine compounds of the present invention may also be deliverable transdermally. The transdermal compositions may take the form of creams, lotions, aerosols and/or emulsions and can be included in a transdermal patch of the matrix or reservoir type as are conventional in the art for this purpose.

The term “capsule” refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

“Tablet” means compressed or molded solid dosage form containing the active ingredients with suitable diluents. The tablet can be prepared by compression of mixtures or granulations obtained by wet granulation, dry granulation or by compaction well known to a person skilled in the art.

“Oral gels” refers to the active ingredients dispersed or solubilized in a hydrophillic semi-solid matrix.

“Powders for constitution” refers to powder blends containing the active ingredients and suitable diluents which can be suspended in water or juices.

“Suitable diluents” are substances that usually make up the major portion of the composition or dosage form. Suitable diluents include sugars such as lactose, sucrose, mannitol and sorbitol, starches derived from wheat, corn rice and potato, and celluloses such as microcrystalline cellulose. The amount of diluents in the composition can range from about 5 to about 95% by weight of the total composition, preferably from about 25 to about 75%, more preferably from about 30 to about 60% by weight.

The term “disintegrants” refers to materials added to the composition to help it break apart (disintegrate) and release the medicaments. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. The amount of disintegrant in the composition can range from about 2 to about 20% by weight of the composition, more preferably from about 5 to about 10% by weight.

“Binders” characterize substances that bind or “glue” powders together and make them cohesive by forming granules, thus serving as the “adhesive” in the formulation. Binders add cohesive strength already available in the diluents or bulking agent. Suitable binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

“Lubricant” refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and D,L-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

“Glidents” are materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

“Coloring agents” are excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

The invention will now be illustrated by a series of Examples which are intended to set forth typical and preferred procedures to be utilized in practice, but which shall not limit the ambit of the claims and the scope of protection.

### EXAMPLES

### A. Synthesis

### Materials and Methods

### Analysis Parameters (HPLC-MS): Method A

### Waters HPLC/MS:

MS detector: ZMD
UV detector: Waters 996 DAD

### Controller: Waters 600

### Autosampler: Waters 2700

Fraction collector: Waters Fraction collector II

### HPLC:

### Column: Supelco Discovery RP-AmideC16

### Solvent A: 10% MeCN/90% Water/0.05% HCOOH

### Solvent B: 100% MeCN

Acetonitrile: Riedel-deHaën; G Chromasolv (34998)

### Water: Mili-Q Academic

Formic Acid Riedel-deHaën; extra pure (27001)
Flow Rate: 3 cm³/min

| | min | B % |
|---|---|---|
| Gradient: | 0.00 | 0 |
| | 0.50 | 0 |
| | 2.00 | 80 |
| | 4.00 | 80 |
| | 4.20 | 0 |
| | 6.00 | 0 |
| Injection: | 5 μg | |

### MS:

### Ionization: ES+/ES−

Source block temp: 120° C.
Desolvation temp: 350° C.

### Desolvation Gas: 400 L/min

### Cone Gas: 100 L/min

### Capillary: 3000 V

### Cone: 25 V

### Extractor: 3 V

### Rf Lens: 0.2 V

### Scan: 120 to 1000 m/z in 1 sec.

Inter-scan delay: 0.1 sec
Analysis parameters (HPLC-MS): Method B

### Waters HPLC/MS:

MS detector: ZMD
UV detector: Waters 996 DAD
Separation module: Waters Alliance 2795

### HPLC:

### Column: Merck Chromolith C18

### Solvent A: Water/0.05% HCOOH

### Solvent B: AcCN/0.05% HCOOH

Acetonitrile: Riedel-deHaën; G Chromasolv (34998)

### Water: Mili-Q Academic

Formic Acid Riedel-deHaën; extra pure (27001)
Flow Rate: 2 ml/min

| | min | B % |
|---|---|---|
| Gradient: | 0.00 | 5 |
| | 0.50 | 5 |
| | 5.50 | 95 |
| | 6.00 | 95 |
| | 6.50 | 5 |
| | 7.00 | 5 |
| Injection: | 3 μg | |

### MS:

### Ionization: ES+/ES−

Source block temp: 120° C.
Desolvation temp: 350° C.

### Desolvation Gas: 400 L/h

### Cone Gas: 100 L/h

### Capillary: 3000 V

### Cone: 25 V

### Extractor: 3 V

### Rf Lens: 0.2 V

### Scan: 120 to 1000 m/z in 1 sec.

Inter-scan delay: 0.1 s

### Method 1

0.50 mmol of corresponding amino derivative and 0.75 mmol of carbonyl chloride or sulfonyl chloride in 10 cm³ N,N dimethylformamide was stirred at 0° C. for 3 hours. Then 100 g of crashed ice and 20 cm³ of saturated NaHCO₃ solution was added and stirred for another one hour. The precipitate was filtered off, washed with cold water and dried at room temperature. Crude materials were recristallized from ethylalcohol, washed with diethyl ether and dried at room temperature.

### Example

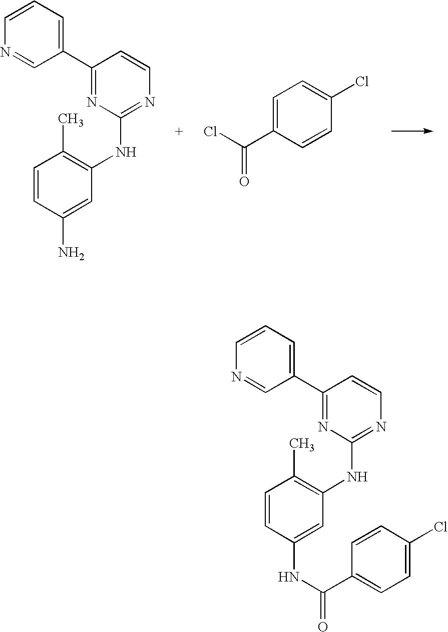

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 5 | A | 2.95 | | 428.06 | 6 | A | 3.10 | | 414.10 |
| 9 | A | 3.12 | | 414.21 | 10 | A | 3.13 | | 400.19 |
| 11 | A | 2.94 | 458.25 | | 12 | A | 2.97 | | 391.25 |
| 13 | A | 2.97 | 398.28 | | 14 | A | 3.13 | | 436.11 |
| 15 | A | 2.93 | 374.14 | | 16 | A | 3.04 | | 426.21 |
| 17 | A | 2.93 | | 470.28 | 18 | A | 2.96 | 407.16 | |
| 19 | A | 2.96 | 412.18 | | 20 | A | 3.12 | | 450.13 |
| 21 | A | 2.91 | 388.20 | | 22 | A | 3.04 | | 440.29 |
| 23 | A | 3.18 | | 464.27 | 24 | A | 3.03 | | 386.33 |
| 25 | A | 3.07 | | 372.16 | 26 | A | 2.85 | | 467.17 |
| 27 | A | 3.00 | 455.23 | | 29 | A | 2.87 | | 405.11 |
| 30 | A | 3.04 | 452.19 | | 31 | A | 2.88 | 412.26 | |
| 32 | A | 3.03 | | 414.24 | 33 | A | 3.78 | | 372.32 |
| 34 | A | 2.96 | | 426.27 | 36 | A | 2.84 | | 372.20 |
| 37 | A | 3.05 | 438.10 | | 38 | A | 3.06 | | 400.13 |
| 41 | A | 3.24 | 466.25 | | 42 | A | 2.93 | 455.23 | |
| 43 | A | 2.90 | | 396.21 | 44 | A | 2.89 | | 391.19 |
| 45 | A | 2.89 | | 456.19 | 46 | A | 2.93 | | 440.16 |
| 47 | A | 2.85 | | 470.13 | 48 | A | 2.90 | | 386.09 |
| 49 | A | 3.19 | | 470.16 | 50 | A | 3.20 | | 405.12 |
| 52 | A | 3.37 | | 414.09 | 53 | A | 3.30 | 388.19 | |
| 54 | A | 3.26 | | 430.09 | 55 | A | 3.18 | 412.14 | |
| 56 | A | 3.23 | 442.17 | | 57 | A | 3.20 | 432.22 | |
| 59 | A | 3.35 | | 400.09 | 60 | A | 3.20 | 398.23 | |
| 61 | A | 3.31 | | 436.14 | 62 | A | 3.19 | 374.19 | |
| 63 | A | 3.33 | | 452.19 | 64 | A | 3.10 | | 453.18 |
| 65 | A | 3.18 | 360.21 | | 66 | A | 3.39 | 418.18 | |
| 67 | A | 3.18 | | 391.19 | 68 | A | 3.23 | | 426.24 |
| 69 | A | 3.36 | 447.13 | | 70 | A | 3.26 | | 380.20 |
| 71 | A | 3.21 | 422.14 | | 72 | A | 3.55 | 437.13 | |
| 73 | A | 3.16 | 368.24 | | 74 | A | 3.25 | 416.19 | |
| 75 | A | 3.21 | 418.20 | | 77 | A | 3.08 | | 416.22 |
| 78 | A | 3.04 | 412.18 | | 79 | A | 2.93 | | 366.16 |
| 80 | A | 3.06 | 398.23 | | 81 | A | 3.06 | | 380.18 |
| 82 | A | 3.01 | | 394.26 | 83 | A | 2.94 | | 380.21 |
| 86 | A | 2.66 | | 381.20 | 87 | A | 2.67 | 369.17 | |
| 90 | A | 2.95 | 398.21 | | 92 | A | 3.10 | | 450.17 |
| 93 | A | 2.95 | | 391.10 | 94 | A | 2.94 | | 380.23 |
| 95 | A | 2.82 | | 366.24 | 96 | A | 3.14 | 418.17 | |
| 98 | A | 2.91 | | 456.18 | 99 | A | 2.92 | | 410.11 |
| 100 | A | 2.98 | | 396.20 | 101 | A | 3.01 | | 380.23 |
| 105 | A | 2.86 | | 391.12 | 106 | A | 2.95 | | 414.06 |
| 107 | A | 3.03 | | 414.16 | 109 | A | 3.06 | | 400.13 |
| 110 | A | 3.30 | | 416.19 | 111 | A | 3.07 | | 456.18 |
| 112 | A | 3.31 | | 450.05 | 113 | A | 3.25 | | 400.07 |
| 114 | A | 3.18 | 398.18 | | 115 | A | 3.19 | | 380.22 |
| 116 | A | 3.10 | | 408.04 | 117 | A | 3.14 | | 450.14 |
| 118 | A | 2.86 | 369.19 | | 119 | A | 3.10 | 374.11 | |
| 120 | A | 2.84 | 368.14 | | 121 | A | 2.69 | | 367.17 |
| 122 | A | 2.93 | | 396.17 | 123 | A | 3.16 | | 426.16 |
| 124 | A | 2.99 | | 426.17 | 125 | A | 2.62 | 340.13 | |
| 126 | A | 2.92 | 340.14 | | 127 | A | 2.70 | 340.15 | |
| 128 | A | 2.71 | | 338.18 | 129 | A | 2.62 | | 338.17 |
| 130 | A | 2.94 | | 338.21 | 133 | A | 2.79 | | 479.17 |
| 134 | A | 2.79 | | 459.18 | 136 | A | 2.73 | | 535.20 |
| 137 | A | 2.74 | | 470.07 | 138 | A | 2.57 | | 446.22 |
| 139 | A | 2.71 | | 487.11 | 140 | A | 2.84 | | 495.19 |
| 141 | A | 2.75 | | 475.21 | 146 | A | 2.81 | | 493.08 |
| 148 | A | 2.75 | | 456.10 | 149 | A | 2.87 | | 481.11 |
| 150 | A | 2.62 | | 432.12 | 151 | A | 2.74 | | 521.09 |
| 152 | A | 2.80 | | 465.05 | 153 | A | 3.25 | | 489.12 |
| 154 | A | 3.21 | | 453.07 | 155 | A | 3.22 | | 487.08 |
| 157 | A | 2.79 | 447.07 | | 158 | A | 2.78 | 463.05 | |
| 160 | A | 2.81 | | 479.04 | 161 | A | 3.35 | 475.11 | |
| 162 | A | 3.16 | | 529.03 | 164 | A | 3.19 | | 464.06 |
| 165 | A | 2.93 | | 440.08 | 166 | A | 3.26 | 471.09 | |
| 175 | A | 2.77 | | 352.15 | 180 | A | 2.91 | | 352.22 |
| 197 | A | 2.96 | | 456.10 | 198 | A | 3.00 | | 396.14 |
| 199 | A | 2.60 | | 367.12 | 200 | A | 3.01 | | 416.12 |
| 201 | A | 2.98 | | 400.11 | 203 | A | 2.80 | | 408.07 |
| 205 | A | 3.03 | 447.10 | | 206 | A | 3.05 | | 456.09 |
| 209 | A | 3.00 | | 436.08 | 210 | A | 3.05 | 454.14 | |
| 211 | A | 3.02 | | 416.13 | 216 | A | 3.26 | | 436.10 |
| 217 | A | 3.29 | 454.17 | | 218 | A | 3.10 | | 436.00 |
| 219 | A | 3.15 | | 452.04 | 220 | A | 2.94 | | 410.11 |
| 222 | A | 3.10 | | 430.11 | 223 | A | 2.85 | | 380.17 |
| 225 | A | 2.96 | | 394.17 | 227 | A | 3.04 | | 466.10 |
| 231 | A | 2.92 | | 386.24 | 234 | A | 2.77 | | 449.16 |
| 235 | A | 3.25 | | 457.20 | 238 | A | 2.77 | | 463.15 |
| 239 | A | 3.29 | | 443.14 | 241 | A | 3.38 | | 459.07 |
| 242 | A | 3.30 | 441.18 | | 243 | A | 3.20 | | 515.16 |
| 244 | A | 3.23 | | 450.15 | 245 | A | 3.32 | | 473.13 |
| 246 | A | 3.42 | | 475.15 | 247 | A | 3.37 | | 455.13 |
| 248 | A | 3.14 | | 426.15 | 251 | A | 2.96 | | 526.12 |
| 257 | A | 2.85 | | 481.14 | 260 | A | 2.92 | | 430.15 |
| 261 | A | 1.62 | | 366.19 | 266 | A | 2.85 | | 381.17 |
| 269 | A | 3.35 | | 430.14 | 270 | A | 3.31 | | 458.00 |
| 272 | A | 3.21 | | 394.17 | 273 | A | 3.29 | | 466.05 |
| 274 | A | 3.22 | | 428.07 | 275 | A | 3.21 | | 410.13 |
| 277 | A | 3.20 | | 434.04 | 278 | A | 3.13 | | 372.22 |
| 280 | A | 2.72 | | 532.01 | 283 | A | 3.01 | 416.06 | |
| 284 | A | 3.21 | | 481.01 | | | | | |

### Method 2

14.00 mmol of corresponding amino derivative and 10.00 mmol of carboxylic acid, 11.00 mmol of 1-hydroxybenzotriazole and 11.10 mmol of N′-(3-dimethylaminopropyl)-N-ethylcarbodiimid hydrochlorid in 120 cm³ N,N dimethylformamide was stirred overnight at room temperature. Then 1000 g of crashed ice was added and stirred further one hour. The precipitate was filtered off, washed with saturated NaHCO₃ solution, water and dried at room temperature. The crude material was refluxed in ethylalcohol for 10 minutes, cooled back and filtered off.

### Example

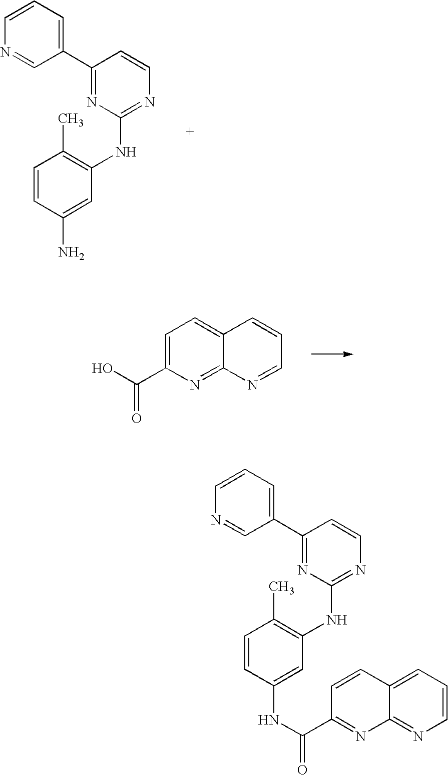

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 88 | A | 2.88 | 420.19 | | 102 | A | 2.93 | 420.15 | |
| 179 | A | 2.84 | | 432.19 | 212 | A | 2.82 | | 432.12 |

### Method 3

7.16 mmol of corresponding amide derivative and 4.96 mmol of Lawesson's reagent in 15 cm³ pyridine was refluxed overnight. Then 100 g of crashed ice was added and stirred for another one hour. The precipitate was filtered off, washed with cold water and dried at room temperature. Crude materials were rechristallized from N,N-dimethylformamide, and washed with acetone.

### Example

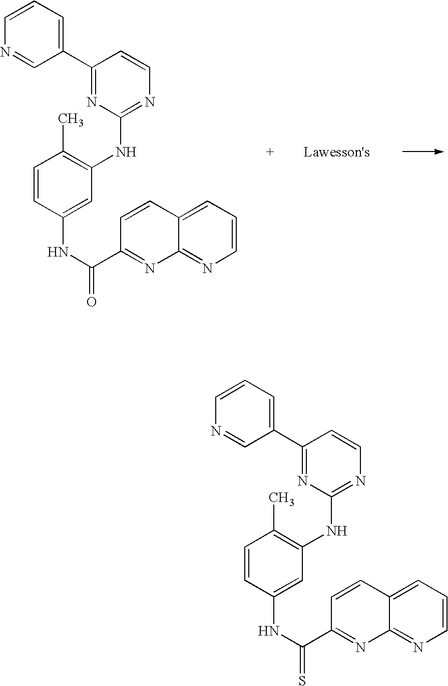

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 89 | A | 3.07 | | 434.17 | 104 | A | 3.13 | | 434.12 |
| 156 | A | 2.61 | | 551.19 | 259 | A | 2.98 | 450.08 | |

### Method 4

4.00 mmol of corresponding chloro derivative and 40.00 mmol of amine derivative was refluxed in 200 cm³ acetonitrile for 6 hours. The reaction mixture was evaporated in vacuum to 100 cm³ and cooled to 0° C., the precipitate was filtered off, washed with diethyl ether and dried at room temperature.

### Example

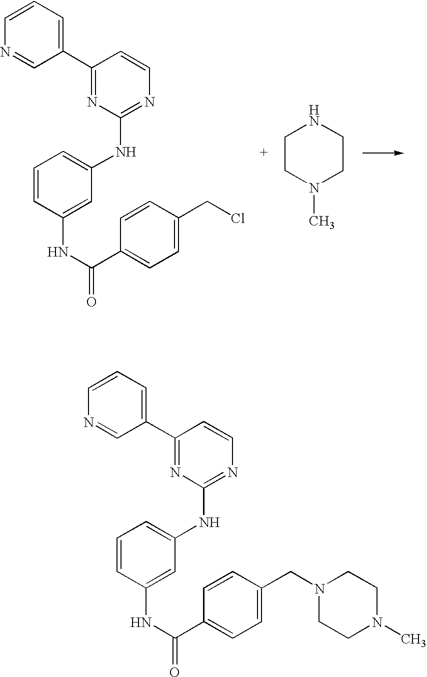

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 7 | A | 2.45 | 480.33 | | 8 | A | 2.45 | 494.31 | |
| 76 | A | 2.61 | | 478.31 | 108 | A | 2.54 | 480.17 | |
| 131 | A | 0.87 | | 402.26 | 171 | A | 0.52 | 404.18 | |
| 172 | A | 1.93 | | 460.11 | 173 | A | 0.56 | 391.15 | |
| 174 | A | 1.48 | | 459.19 | 182 | A | 2.56 | | 478.29 |
| 183 | A | 2.57 | | 460.15 | 184 | A | 1.46 | | 389.20 |
| 185 | A | 2.52 | | 459.23 | 186 | A | 1.64 | 404.20 | |
| 187 | A | 1.68 | | 389.19 | 188 | A | 2.63 | | 460.21 |
| 189 | A | 2.62 | | 459.17 | 190 | A | 0.75 | 418.21 | |
| 191 | A | 2.58 | | 473.17 | 192 | A | 2.53 | | 474.18 |
| 193 | A | 1.03 | | 403.15 | 195 | A | 0.36 | | 416.19 |
| 196 | A | 0.65 | | 403.15 | 202 | A | 0.77 | | 473.18 |
| 204 | A | 0.88 | | 474.22 | 207 | A | 1.13 | | 416.20 |
| 208 | A | 1.27 | | 403.28 | 213 | A | 0.49 | 494.22 | |
| 214 | A | 2.55 | | 473.25 | 215 | A | 2.58 | | 474.21 |
| 224 | A | 0.61 | 404.19 | | 226 | A | 0.63 | | 389.18 |
| 228 | A | 2.32 | | 459.17 | 229 | A | 1.74 | | 460.20 |
| 236 | A | 2.44 | | 543.13 | 249 | A | 2.65 | | 537.17 |
| 252 | A | 2.42 | | 557.09 | 254 | A | 1.02 | 480.27 | |
| 262 | A | 0.34 | 432.21 | | 263 | A | 0.75 | | 488.21 |
| 264 | A | 1.23 | | 402.19 | 265 | A | 1.35 | | 389.27 |
| 267 | A | 2.57 | | 459.17 | 268 | A | 2.60 | | 460.18 |
| 271 | A | 2.46 | | 431.14 | 276 | A | 2.56 | | 492.20 |
| 285 | A | 2.49 | | 478.10 | | | | | |

### Method 5

0.76 mmol of dimethoxy derivative and 3.00 mmol of boron tribromide in 30 cm³ abs. dichloromethane was stirred for 3 hours at −70° C. Then 50 g of crashed ice and 10 cm³ of saturated NaHCO₃ solution was added to quench the reaction and stirred for another one hour. Then this mixture was extracted three times with 100-100 cm³ dichloromethane. Organic phase was dried over MgSO₄ and evaporated to dryness. Residue was stirred for 10 minutes in 20 cm³ diethyl ether filtered off and air dried.

### Example

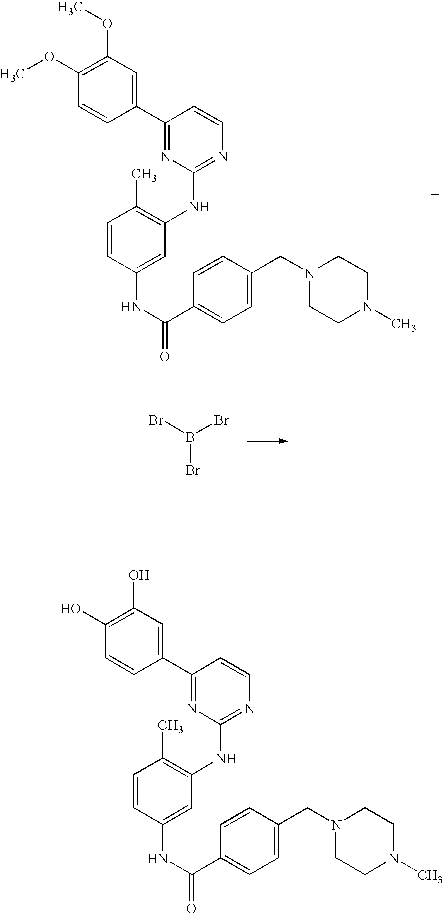

The following compounds have been synthesized according to this method:

| | | | | | | | | | |
|---|---|---|---|---|---|---|---|---|---|
| | LCMS | Retention | | | Comp | LCMS | Retention | | |
| Comp No. | Method | Time | M⁺ | M⁻ | No. | Method | Time | M⁺ | M⁻ |

| | | | |
|---|---|---|---|
| 159 | A | 2.56 | 523.11 |

### Method 6

2.00 mmol of corresponding amino derivative, 2.20 mmol of 4-chloro-3-methyl-isoxazolo[5,4-d]pyrimidine and 0.4 cm³ of triethylamine in 40 cm³ ethylalcohol was refluxed for 2 hours. The reaction mixture was evaporated under reduced pressure to 10 cm³ and cooled to 0° C., the precipitate was filtered off, washed with diethyl ether and dried at room temperature.

### Example

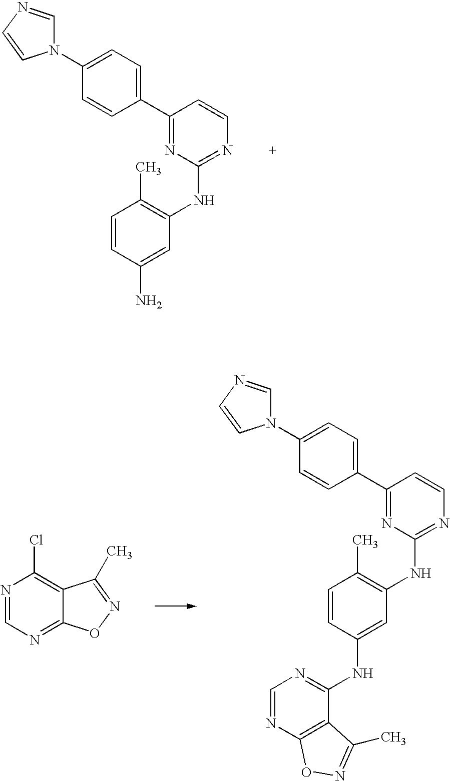

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 167 | A | 2.88 | | 395.15 | 168 | A | 2.80 | | 395.14 |
| 170 | A | 2.78 | | 395.20 | 176 | A | 2.90 | | 409.15 |
| 194 | A | 2.82 | | 409.10 | 221 | A | 3.05 | | 395.09 |
| 233 | A | 2.68 | | 460.17 | 237 | A | 3.11 | | 468.15 |
| 240 | A | 2.66 | | 474.13 | 250 | A | 3.19 | | 454.08 |

### Method 7

1.50 mmol of 3-methyl-isoxazolo[5,4-d]pyrimidine derivative, which was prepared according to METHOD 6, and 0.060 g of 10% palladium on activated carbon was hydrogenated in 100 cm³ ethylalcohol:tetahydrofuran=1:1 under atmospheric pressure at room temperature for 6 hours. The reaction mixture was evaporated under reduced pressure to dryness. Crude materials were recristallized from ethylalcohol, washed with diethyl ether and dried at room temperature.

### Example

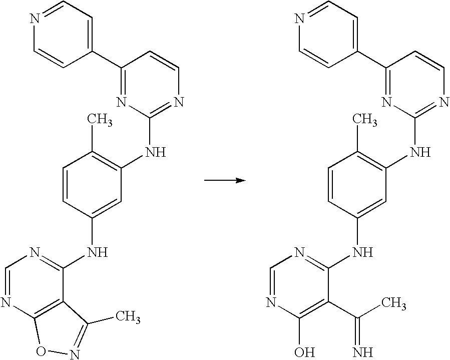

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 169 | A | 0.73 | | 397.15 | 177 | A | 2.64 | | 411.18 |
| 178 | A | 2.58 | | 411.14 | 181 | A | 2.80 | 399.23 | |

### Method 8

0.50 mmol of ester derivative and 0.52 mmol of NaOH in 20 cm³ methylalcohol:water=1:1 was stirred overnight at room temperature. The excess of methylalcohol was evaporated from the reaction mixture under reduced pressure. The aqueous residue was acidified with 1 M hydrochloric acid solution in water to pH=4 and cooled to 0° C. The precipitate was filtered off, washed with water. The crude product was suspended in acetonitrile, stirred for 10 minutes and filtered off, washed with diethyl ether, dried at room temperature.

### Example

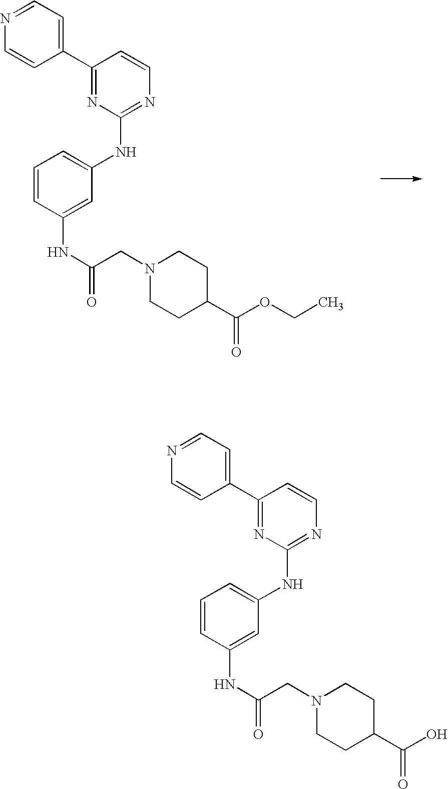

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 230 | A | 0.72 | | 431.17 | 302 | B | 2.44 | 293.07 | |
| 304 | B | 3.35 | 327.07 | | | | | | |

### Method 9

40 mmol of the appropriate dimethylamino-propenon derivative and 40 mmol of the nitrophenyl-guanidine salt were suspended with 60 cm³ of 2-propanol and the contents were stirred for 5-10 minutes. Then 44.1 mmol of sodium hydroxide were added to them and the reaction mixture was stirred and refluxed for 8-12 hours. The reaction mixture was cooled to 0° C. The product was filtered and washed with 2-propanol and then the crude material was stirred with 300 cm³ of water for 30 minutes. The product was filtered again, washed with water and then with ethanol and with diethyl ether and dried at room temperature in the end.

### Example

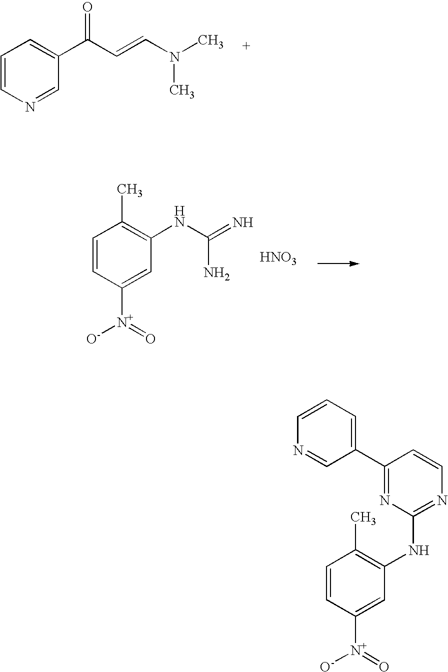

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 1 | A | 3.30 | 308.15 | | 2 | A | 3.00 | | 292.22 |
| 40 | A | 2.93 | 292.20 | | 58 | A | 2.76 | | 371.26 |
| 91 | A | 0.37 | 264.26 | | 97 | A | 0.72 | 264.24 | |
| 103 | A | 0.35 | 264.21 | | 135 | A | 2.77 | 359.16 | |
| 142 | A | 0.64 | 329.20 | | 143 | A | 2.65 | | 547.07 |
| 144 | A | 3.23 | | 365.12 | 145 | A | 3.26 | 353.16 | |
| 147 | A | 2.65 | 323.17 | | 232 | A | 3.24 | 537.13 | |
| 253 | A | 1.49 | 351.16 | | 255 | A | 2.57 | 329.17 | |
| 256 | A | 2.49 | 323.24 | | 258 | A | 3.02 | | 281.18 |
| 281 | A | 2.99 | 308.20 | | 282 | A | 3.45 | | 316.10 |
| 288 | A | 1.75 | | 405.09 | 291 | B | 4.59 | 317.05 | |
| 293 | B | 3.61 | | 315.10 | 295 | B | 3.23 | 321.22 | |
| 309 | B | 4.42 | 329.05 | | 313 | B | 4.62 | | 349.10 |

### Method 10

10 mmol of the appropriate aromatic nitro compound were added to a solution of 40 mmol of tin(II) chloride dihydrate and 25 cm³ of concentrated hydrochloric acid at 50° C. while stirring thoroughly. The solution warmed to 90-100° C. and the product precipitated. The contents were stirred and warmed at 100° C. for another 30 minutes. After cooling, the contents were poured into a mixture of ice, water and about 35 g of potassium carbonate bit by bit while stirring (it must be alkaline in the end). The mixture was extracted three times with 150 cm³ of ethyl acetate, the combined organics were washed with water/brine and dried over magnesium sulfate. Evaporation of the solvent gave the crude product, which was purified by recristallization from 20-30 cm³ of dichloromethane to afford the product as an almost white solid.

### Example

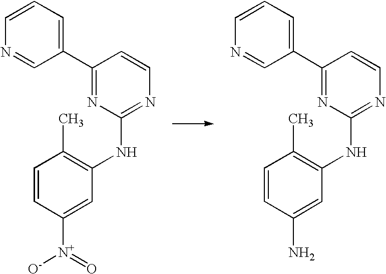

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 3 | A | 0.35 | 264.22 | | 4 | A | 0.35 | 278.23 | |
| 28 | A | 1.05 | 278.19 | | 35 | A | 0.33 | 278.24 | |
| 39 | A | 0.36 | 264.21 | | 51 | A | 2.64 | 264.23 | |
| 132 | A | 0.51 | 343.24 | | 163 | A | 2.57 | 337.17 | |

### Method 11

2 mmol of the appropriate aromatic amine in toluene were stirred in inert gas atmosphere and 2 mmol of trichloromethyl chloroformate were added and the contents were refluxed for 2 hours. Then the contents of the flask were cooled to room temperature and 2 mmol of the second molecule of amine were added and the reaction mixture was refluxed for an hour again. After cooling the precipitate was filtered off, washed with water, sat. sodium hydrogencarbonate solution and water, dried at room temperature. The crude material was recrystallized from acetone, the product was washed with ice-cool acetone and ether, dried at room temperature.

### Example

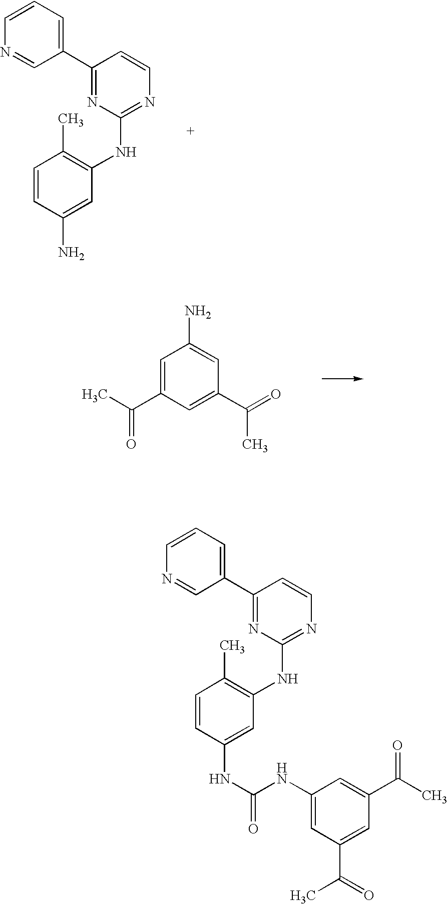

The following compounds have been synthesized according to this method:

| | | | | | | | | | |
|---|---|---|---|---|---|---|---|---|---|
| | LCMS | Retention | | | Comp | LCMS | Retention | | |
| Comp No. | Method | Time | M⁺ | M⁻ | No. | Method | Time | M⁺ | M⁻ |

| | | | |
|---|---|---|---|
| 84 | A | 3.04 | 479.19 |

### Method 12

0.83 mmol of aminoguanidine salt was stirred with ethanol and conc. hydrochloric acid solution at room temperature for 15 minutes, and 0.4 mmol of the appropriate of aromatic acetophenone was added, then the solution was refluxed for about 24 hours. The reaction mixture was cooled to 0° C. and the precipitate was filtered off, washed with ethanol, ethyl acetate and dried at room temperature.

### Example

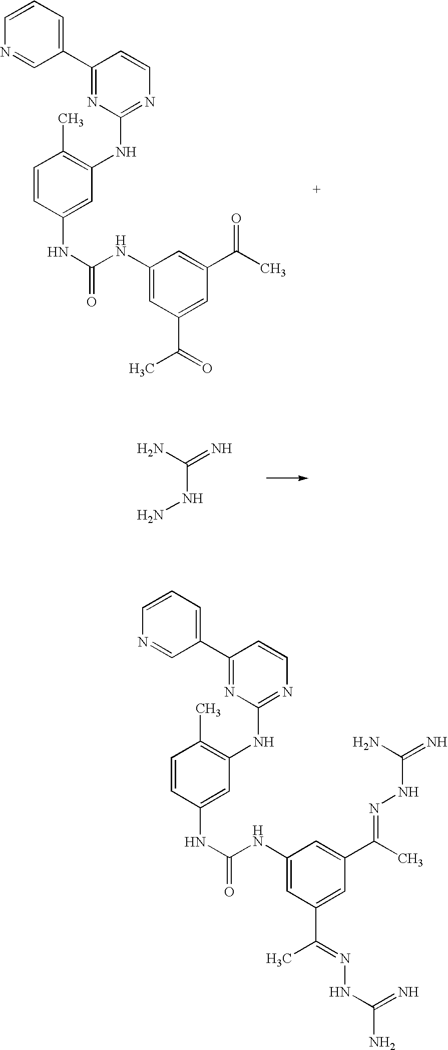

The following compounds have been synthesized according to this method:

| | | | | | | | | | |
|---|---|---|---|---|---|---|---|---|---|
| | LCMS | Retention | | | Comp | LCMS | Retention | | |
| Comp No. | Method | Time | M⁺ | M⁻ | No. | Method | Time | M⁺ | M⁻ |

| | | | |
|---|---|---|---|
| 85 | A | 2.54 | 591.25 |

### Method 13

1 mmol of the appropriate 2-chloro-pyridine derivative and 20 mmol of primer or secondary amine compound were stirred in inert gas atmosphere at 140-160° C. for 2-6 hours. The contents of the flask were cooled and the crude product was rubbed with ice-cool water. The material was washed with water, sodium hydrogencarbonete solution and water, dried at room temperature. If it was necessary the product was recrystallized from ethanol (isopropanol/acetonitrile).

### Example

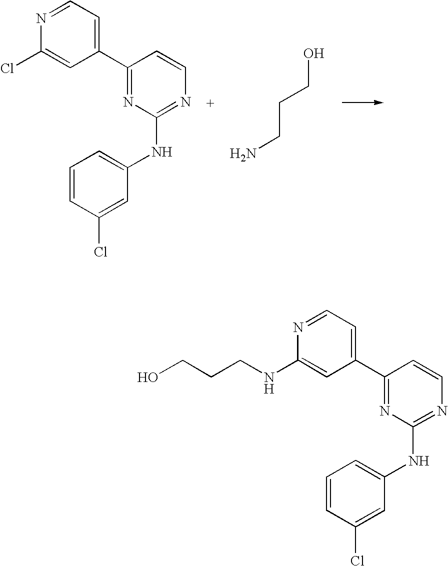

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 279 | A | 2.60 | | 354.16 | 286 | A | 0.39 | 397.16 | |
| 287 | A | 1.41 | 382.16 | | 290 | B | 2.76 | 356.10 | |
| 292 | B | 2.59 | 375.03 | | 297 | B | 2.86 | | 388.13 |
| 298 | B | 2.84 | 376.02 | | 299 | B | 2.95 | 418.02 | |
| 300 | B | 2.52 | 440.01 | | 301 | B | 2.41 | 472.23 | |
| 303 | B | 2.11 | 423.16 | | 305 | B | 2.27 | 366.18 | |
| 310 | B | 2.63 | 354.17 | | 311 | B | 2.77 | 396.22 | |
| 312 | B | 2.68 | 368.23 | | | | | | |

### Method 14

2 mmol of pivaloyl-protected compound was warmed to 70° C. in 40 cm³ 70% methanesulphonic acid for 5 hours. Then it was poured onto 100 g crashed ice, pH was set to 10 and extracted several times with 50 cm³ chloroform. Organic phases were combined, dried over magnesium sulfate and evaporated to dryeness. Residue was stirred for 10 minutes in 20 cm³ diethyl ether filtered off and air dried.

### Example

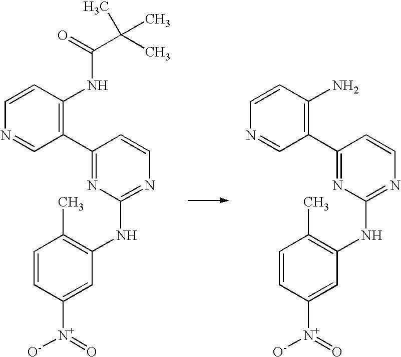

The following compounds have been synthesized according to this method:

| | | | | | | | | | |
|---|---|---|---|---|---|---|---|---|---|
| | LCMS | Retention | | | Comp | LCMS | Retention | | |
| Comp No. | Method | Time | M⁺ | M⁻ | No. | Method | Time | M⁺ | M⁻ |

| | | | |
|---|---|---|---|
| 289 | A | 1.35 | 323.11 |

### Method 15

8 mmol of the appropriate pyridyl-pyrimidine derivative were stirred in dichloromethane, 8 mmol of 3-chloro-perbenzoic acid were added, the contents were stirred at room temperature for 3 hours. The crude product was precipitated this time. The suspension was diluted with ether to double volume and the material was filtered off, washed with ether and dried at room temperature.

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 294 | B | 3.39 | 333.16 | | 296 | B | 3.13 | 337.15 | |

### Method 16

1 mmol of the appropriate carboxylic acid chloride was dissolved in anhydrous dichloromethane and the solution was cooled below 0° C., then triethylamine and a primer or secondary amine were dropped into it, and the solution was stirred for an hour. The solution was washed with water two times and dried on anhydrous magnesium sulfate. The solvent was evaporated under reduced pressure. The product was washed on a filter with ether or acetonitrile. If it was necessary the material was recrystallized from ethanol (acetonitrile).

### Example

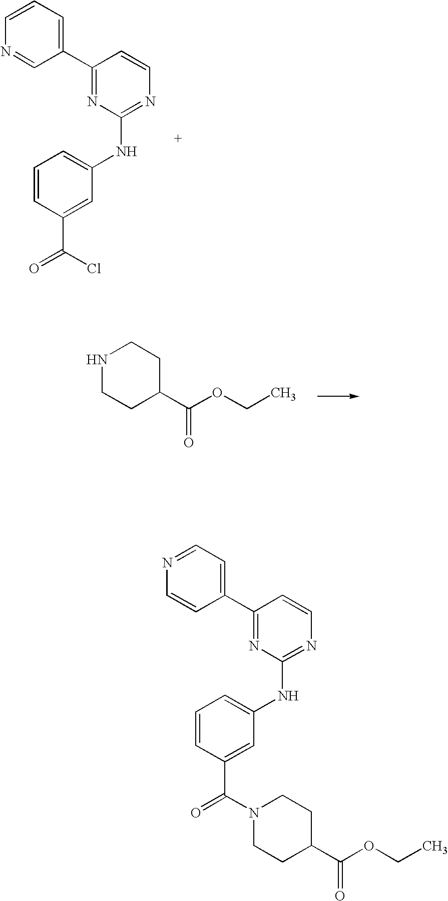

The following compounds have been synthesized according to this method:

| Comp | LCMS | Retention | | | Comp | LCMS | Retention | | |
|---|---|---|---|---|---|---|---|---|---|
| No. | Method | Time | M⁺ | M⁻ | No. | Method | Time | M⁺ | M⁻ |
| 306 | B | 2.88 | 432.20 | | 308 | B | 2.80 | 436.29 | |

### Method 17

3 mmol of the appropriate carboxylic acid were refluxed in 80 cm³ of methanol in presence of conc. sulfuric acid for 16-24 hours. The half volume of the solvent was distilled off and the solution was diluted with ethyl acetate. The solution was washed with sodium chloride solution two times, and dried over anhydrous magnesium sulfate. The solvent was evaporated under reduced pressure. The product was recrystallized from a mixture of dichloromethane and methanol.

### Example

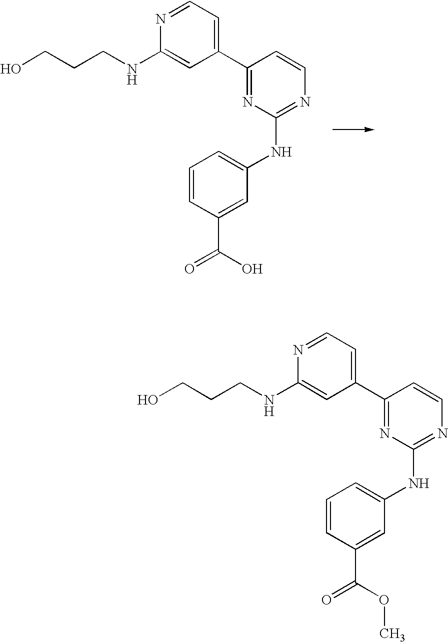

The following compounds have been synthesized according to this method:

| | | | | | | | | | |
|---|---|---|---|---|---|---|---|---|---|
| | LCMS | Retention | | | Comp | LCMS | Retention | | |
| Comp No. | Method | Time | M⁺ | M⁻ | No. | Method | Time | M⁺ | M⁻ |

| | | | |
|---|---|---|---|
| 307 | B | 2.53 | 380.15 |

### B. Biochemical Assay

1. Cell Culture and Expression of 3F4-tagged PrP (3F4-ScN2a)

PrP^{Sc}- and PrP^{c}-transfected mouse neuronal cells (N2A) were cultured in MEM (Minimum Essential Medium, Life Technologies) supplemented with 10% fetal calf serum at 37° C. and 5% CO₂ to obtain ˜6×10⁶ cells per tissue culture flask. The mouse neuroblastoma cell line 3F4-ScN2a represents a stably transfected clone of ScN2a cells (PrP^{Sc} infected N2a cells) which overexpress 3F4-epitope-tagged murine PrP. Residues 109 and 112 of murine PrP were replaced by methionine to introduce the epitope for reactivity with the monoclonal anti-PrP antibody 3F4. Cells were maintained in Dulbecco's modified Eagle's (DMEM) or Opti-MEM medium containing 10% fetal calf serum, antibiotics and glutamin. For generation of stable transfectants we used the vector pcDNA3.1/Zeo (Invitrogen; Leek, The Netherlands). Lipofection of cells with recombinant plasmids was done using standard procedures and recombinant clones were selected by addition of 300 μg Zeocin/ml medium.

2. Treatment of Cells with Inhibitors

All tested compounds were solubilized in DMSO (dimethylsulfoxide), and prepared as 10 mM stock solutions. The drugs were applied to the cells described above for three days in final concentrations between 5 and 20 μM.

### 3. Immunoblot and Proteinase K (PK) Analysis

Confluent cell cultures were lysed in cold lysis buffer (10 mM Tris-HCl, pH 7.5; 100 mM NaCl; 10 mM EDTA; 0.5% Triton X-100; 0.5% DOC) (EDTA: ethylene diamine tetraacetate; Triton X-100: t-octylphenoxypolyethoxyethanol; DOC: deoxycholic acid). Postnuclear lysates were split between those with and without proteinase K digestion. Samples without proteinase K digestion were supplemented with proteinase inhibitors (5 mM PMSF, 0.5 mM Pefabloc, and aprotinin) (PMSF: phenylmethylsulfonyl fluoride) and directly precipitated with ethanol. Samples for proteinase K digestion were incubated with 20 μg/ml proteinase K for 30 min at 37° C.; digestion was stopped with proteinase inhibitors, and samples were ethanol precipitated. After centrifuging for 30 min at 3,500 rpm the pellets were redissolved in TNE buffer (10 mM Tris-HCl pH7.5, 100 mM NaCl, 1 mM EDTA) and gel loading buffer was then added. After boiling for 5 min an aliquot was analyzed on 12.5% PAGE. For Western blot analysis, the proteins were electrotransferred to PVDF membranes (polyvinylidendifluorid). The membrane was blocked with 5% non-fat dry milk in TBST (0.05% Tween 20, 100 mM NaCl, 10 mM Tris-HCl, pH 7.8) (Tween 20: polyoxyethylenesorbitan monolaurate; Tris-HCl: Tris-(hydroxymethyl)-aminomethane-hydrochloride), incubated overnight with the primary antibody at 4° C. and stained using the enhanced chemiluminescence blotting kit from Amersham Corporation. Specific immuno-staining of the PrP^{c} and PrP^{Sc} forms were obtained with the prion protein specific antibody 3F4 (Signet Pathologies, U.S.A.).

### 4. Results

Determination of the amount of the pathogenic form of the prion protein PrP^{Sc} upon treatment of prion infected cells with different types of small molecule protein kinase inhibitors resulted in the identification of a compound class of pyridylpyrimidine derivatives exemplified by the compound 4-(4-Methylpiperazin-1-ylmethyl)-N-[4-methyl-3-(4-pyridin-3-yl-pyrimidin-2-ylamino)-phenyl]-benzamide (compound 53) and compounds 4, 5, and 37.

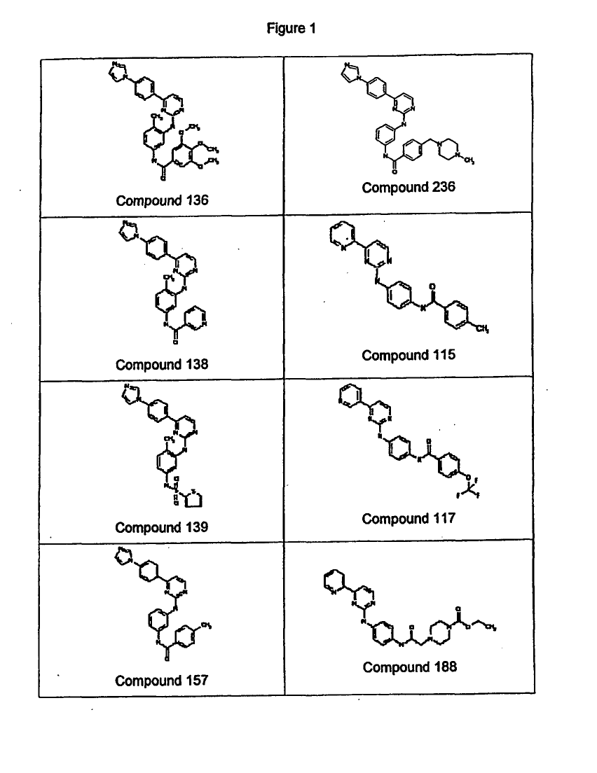

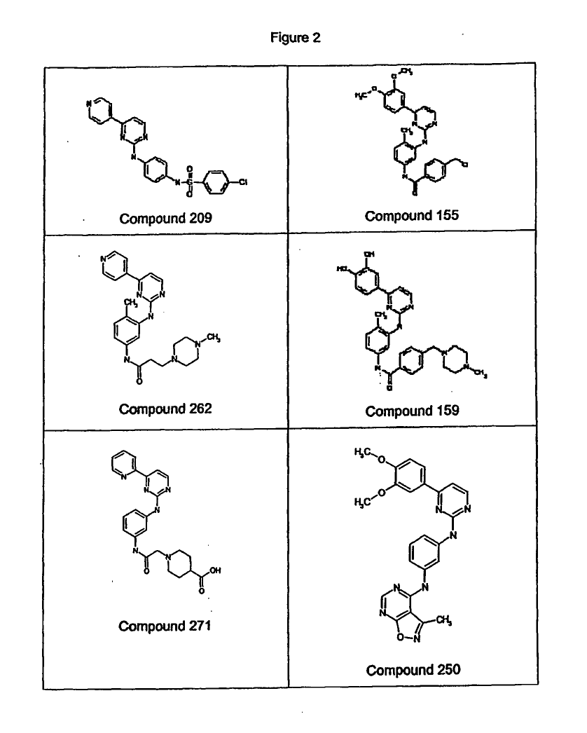

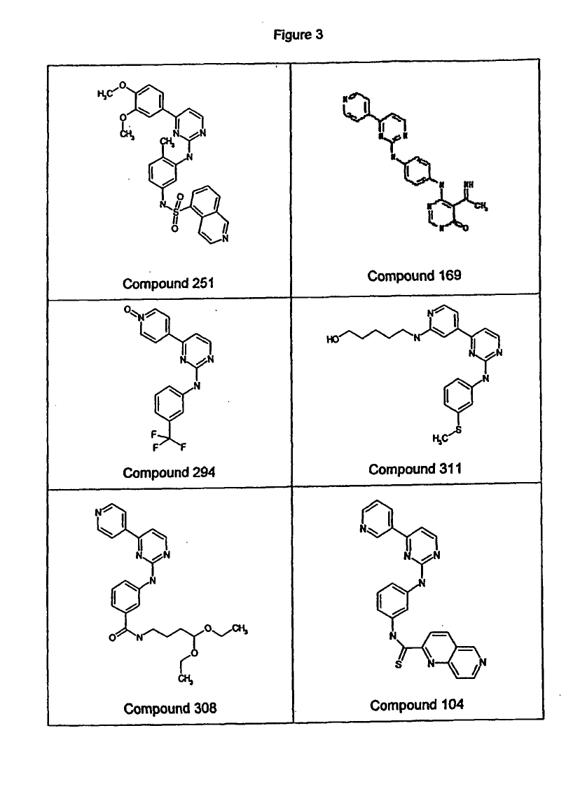

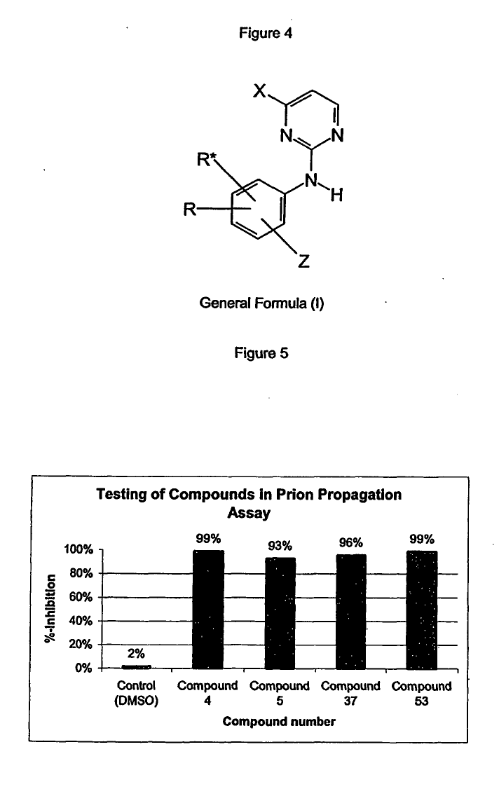

These compounds significantly reduced the amount of PrP^{Sc} in prion infected cells in a concentration range between 5 and 20 μM (final concentration). As shown in FIG. 5 the selected compounds 4, 5, 37, and 53 inhibit almost completely the activity of prion propagation within said concentration range.

The compounds did not show any toxic effects on the cells in these concentrations. Therefore these molecules described herein serve as potential inhibitors for the medical intervention of prion diseases such as transmissible spongiform encephalitis (TSE) infections which include Bovine spongiform encephalitis (BSE) or the new variant of Creutzfeld Jakob disease (vCJK).

What is claimed is:

1. A compound of formula (I): 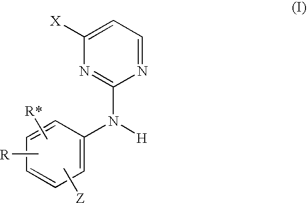
wherein:
R and R* represent independently of each other —H, —OCH₃, —CF₃, —CH₃, —C₂H₅, —R′, —R¹⁷;
R′, R″, R′″ and R″″ represent independently of each other —H, —F, —Cl, —Br, —I, —CN, —OH, —OCH₃, —OC₂H₅, —OCF₃, —NH₂, —NO₂, —N(CH₃)₂, —N(C₂H₅)₂, —SH, —SO₃H, —COOH, —COOCH₃, —COOC₂H₅, —CONH₂;
R¹, R², R³, R⁴, R^{1′}, R^{2′}, and R^{3′} represent independently of each other —H, —R′, —OH, —SH, —OCH₃, —OC₂H₅, —SCH₃, —NH₂, —NO₂, —NH(CH₃), —N(CH₃)₂, —COOH, —COOCH₃, —OCF₃, —CH₃, —C₂H₅, —C₃H₇, —CH(CH₃)₂, —R¹², 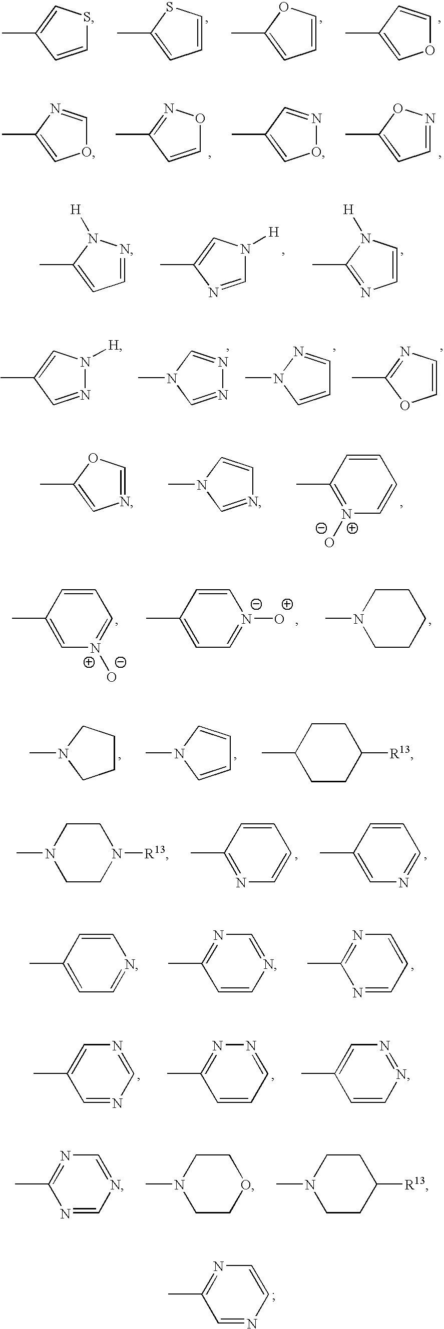
R⁵ represents —H, —R⁴, —CH₂R³, —C₂H₄R³, —C₃H₆R³, —C₄H₈R³, —CHR³R⁴, —CH₂—CHR³R⁴, —C₂H₄—CHR³R⁴, —C₃H₆—CHR³R⁴, —R¹¹, —R¹³, 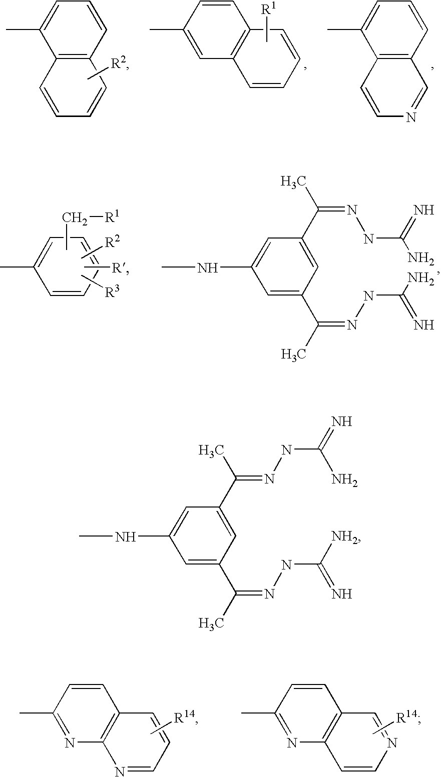
R⁶, R⁷, R⁸ and R⁹ represent independently of each other —H, —R′, —R¹, —CH₂R¹, —R¹²;
R¹⁰, R¹¹, R¹⁷, R¹⁸ and R¹⁹ represent independently of each other —H, —R′, —CH₃, —C₂H₅, —CH═CH₂, —C≡CH, —C₃H₇, -cyclo-C₃H₅, —CH(CH₃)₂, —CH₂—CH═CH₂, —C(CH₃)═CH₂, —CH═CH—CH₃, —C≡C—CH₃, —CH₂—C≡CH, —C₄H₉, -cyclo-C₄H₇, —CH₂—CH(CH₃)₂, —CH(CH₃)—C₂H₅, —C(CH₃)₃, —C₅H₁₁, -cyclo-C₅H₉, —C₆H₁₃, -cyclo-C₆H₁₁, -Ph, —C(R′)₃, —CR′(R″)₂, —CR′(R″)R′″, —C₂(R′)₅, —CH₂—C(R′)₃, —CH₂—CR′(R″)₂, —CH₂—CR′(R″)R′″, —C₃(R′)₇, —C₂H₄—C(R′)₃, —CH(R′)—CH(R″)—CH₂—R′″, —CH₂—R′, —C₂H₄—R′, —C₃H₆—R′, —C₄H₈—R′, —C₅H₁₀—R′, —C₆H₁₂—R′;
R¹² and R¹³ represent independently of each other —H, —F, —Cl, —Br, —I, —CH₂F, —CH₂Cl, —CH₂Br, —CH₂I, —CH₂R³, —OH, —OCH₃; —OC₂H₅, —NH₂, —NH(CH₃), —N(CH₃)₂, —N(C₂H₅)₂, —OCF₃, —CH₃, —C₂H₅, —C₃H₇, —R^{1O}, —NH(R¹⁰), —NH(R¹¹), —N(R¹⁰)₂, —NR¹⁰R¹¹, —OR¹⁰, —OR¹¹, —CO—R¹⁰, —COOH, —COOCH₃, —COOC₂H₅, —COOR¹⁰, —OOCR¹⁰, —SO₃H, —SO₃R¹⁰, —SO₂H, —SO₂R¹⁰, —SO₂—CH₃, —CO—CH₃, —OOC—CH₃, —OOC—C₂H₅, —CONH₂, —CONH(R¹⁰), —CON(R¹⁰)₂, —CONR¹⁰R¹¹, —NH—CO—R¹⁰, —NH—CO—CH₃, —NH—CO—C₂H₅, —NH—CO—C(CH₃)₃, —NH—CO—OCH₃, —NH—CO—NH₂;
R¹⁴ and R¹⁵ represent independently of each other —H, —R^{1′}, —F, —Cl, —Br, —I, —CH₂F, —CH₂Cl, —CH₂Br, —CH₂I, —CH₂R³, —OH, —OCH₃, —OC₂H₅, —NH₂, —NH(CH₃), —N(CH₃)₂, —N(C₂H₅)₂, —OCF₃, —CH₃, —C₂H₅, —C₃H₇, —R¹⁸, —NH(R¹⁸), —NH(R¹⁹), —N(R¹⁸)₂, —NR¹⁸R¹⁹, —OR¹⁸, —CO—R¹⁸, —COOH, —COOCH₃, —COOC₂H₅, —COOR¹⁸, —OOCR¹⁸, —SO₃H, —SO₃R¹⁸, —SO₂H, —SO₂R¹⁸, —SO₂—CH₃, —CO—CH₃, —OOC—CH₃, —OOC—C₂H₅, —CONH₂, —CONH(R¹⁸), —CON(R¹⁸)₂, —CONR¹⁸R¹⁹, —NH—CO—R¹⁸, —NH—CO—CH₃, —NH—CO—C₂H₅, —NH—CO—C(CH₃)₃, —NH—CO—OCH₃, —NH—CO—NH₂, —CR^{1′}(R^{2′})R^{3′}, —CH₂—CR^{1′}(R^{2′})R^{3′}, —CHR^{1′}—CH₂R^{2′}, —CH(R¹′)—CH(R^{2′})—CH₂—R^{3′}, —CH₂—R^{1′}, —C₂H₄—R^{1′}, —C₃H₆—R^{1′}, —C₄H₈—R^{1′}, —C₅H₁₀—R^{1′}, —C₆H₁₂—R^{1′};
X represents 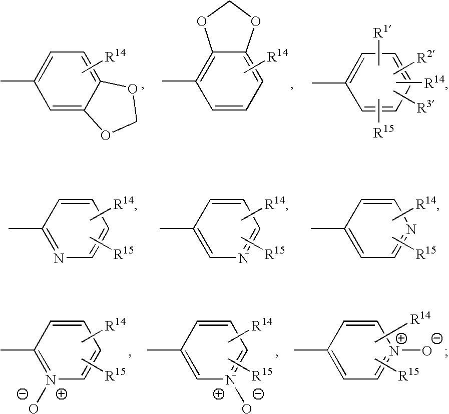
Z represents —NH—CO—R⁵, —CO—NH—R⁵, —NH—CS—R⁵, —NH—SO₂—R⁵, —NH₂, —NO₂, —OCH₃, —SCH₃, —CF₃, —COOH, —COOCH₃, —COOC₂H₅, 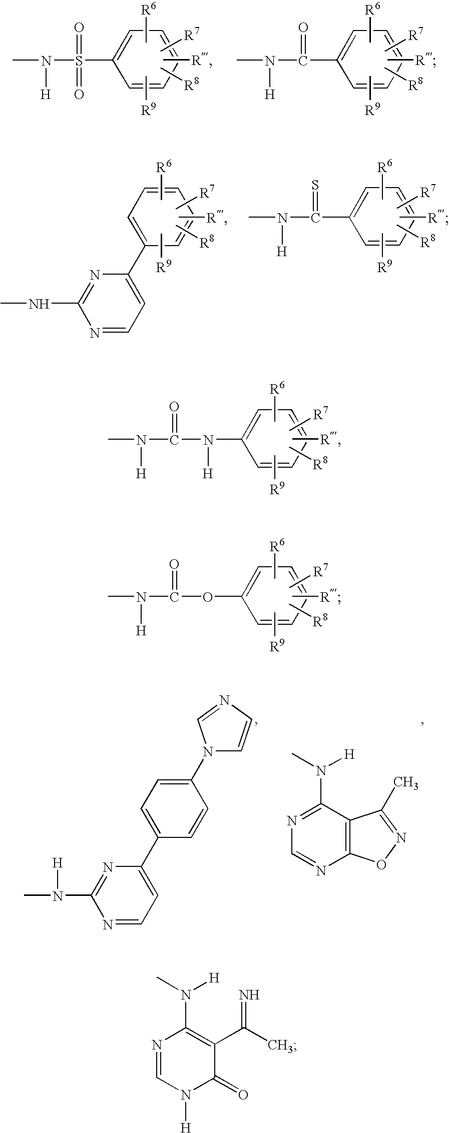
and/or pharmaceutically acceptable salts thereof;
and wherein the following compounds are excluded:
1-(4-{4-[4-(4-Imidazol-1-ylphenyl)-pyrimidin-2-ylamino]-benzoyl}-piperazin-1-yl)ethanone,
4-[4-(4-Imidazol-1-ylphenyl)-pyrimidin-2-ylamino]-benzamide,
2-(3-Fluorophenylamino)-4-(4-imidazol-1-ylphenyl)-pyrimidine-5-carbonitrile.

2. The compound according to claim 1, wherein the compound has the formula (Ic): 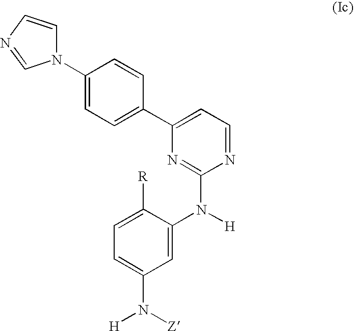
wherein
Z′ is

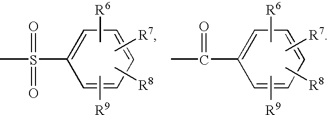

3. The compound according to claim 1, wherein the compound has the formula (Ih): 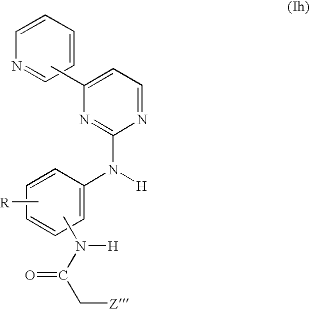
wherein Z′″ represents —R¹, —R⁵, and —R¹³, provided that Z′″ is not —H or CₙH₂ₙ₊₁ with n being an integer between 1 and 6.

4. The compound according to claim 1, wherein the compound has the formula (In): 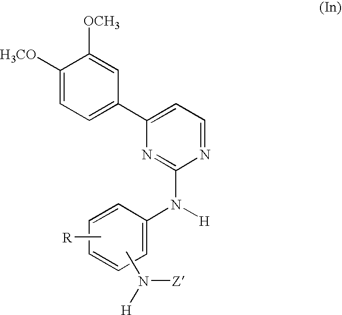
wherein
Z′ is

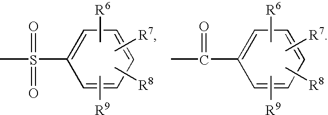

5. The compound according to claim 1, wherein the compound has the formula (Io): 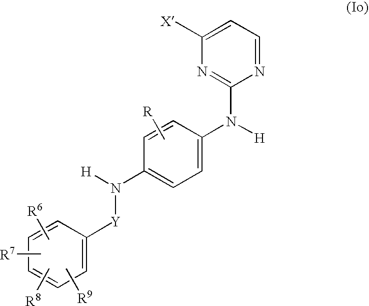
wherein Y represents the residue —C(═O)— or —SO₂—, and X′ represents 2-pyridyl or 4-pyridyl.

6. The compound according to claim 1, wherein the compound is selected from the group consisting of:
Compound 58 [4-(4-Imidazol-1-yl-phenyl)-pyrimidin-2-yl]-(2-methyl-5-nitro-phenyl)-amine;
Compound 93 4-Cyano-N-[4-(4-pyridin-3-yl-pyrimidin-2-ylamino)-phenyl]-benzamide;
Compound 96 Naphthalene-2-carboxylic acid [4-(4-pyridin-3-yl-pyrimidin-2-ylamino)-phenyl]-amide;
Compound 97 N-(4-Pyridin-2-yl-pyrimidin-2-yl)-benzene-1,4-diamine;
Compound 98 3,4,5-Trimethoxy-N-[4-(4-pyridin-3-yl-pyrimidin-2-ylamino)-phenyl]-benzamide;
Compound 99 Thiophene-2-sulfonic acid [4-(4-pyridin-3-yl-pyrimidin-2-ylamino)-phenyl]-amide;
Compound 100 2-Methoxy-N-[4-(4-pyridin-3-yl-pyrimidin-2-ylamino)-phenyl]-benzamide;
Compound 101 4-Methyl-N-[4-(4-pyridin-3-yl-pyrimidin-2-ylamino)-phenyl]-benzamide;
Compound 102 [1,6]Naphthyridine-2-carboxylic acid [3-(4-pyridin-3-yl-pyrimidin-2-ylamino)-phenyl]-amide;
Compound 103 N-(4-Pyridin-4-yl-pyrimidin-2-yl)-benzene-1,4-diamine;
Compound 104 [1,6]Naphthyridine-2-carbothioic acid [3-(4-pyridin-3-yl-pyrimidin-2-ylamino)-phenyl]-amide;
Compound 105 4-Cyano-N-[4-(4-pyridin-4-yl-pyrimidin-2-ylamino)-phenyl]-benzamide;
Compound 106 4-Chloromethyl-N-[4-(4-pyridin-4-yl-pyrimidin-2-ylamino)-phenyl]-benzamide;
Compound 107 4-Chloromethyl-N-[4-(4-pyridin-3-yl-pyrimidin-2-ylamino)-phenyl]-benzamide;
Compound 108 4-(4-Methyl-piperazin-1-ylmethyl)-N-[4-(4-pyridin-3-yl-pyrimidin-2-ylamino)-phenyl]-benzamide;
Compound 109 4-Chloro-N-[4-(4-pyridin-3-yl-pyrimidin-2-ylamino)-phenyl]-benzamide;
Compound 110 Naphthalene-2-carboxylic acid [4-(4-pyridin-2-yl-pyrimidin-2-ylamino)-phenyl]-amide;
Compound 111 3,4,5-Trimethoxy-N-[4-(4-pyridin-2-yl-pyrimidin-2-ylamino)-phenyl]-benzamide;
Compound 112 N-[4-(4-Pyridin-2-yl-pyrimidin-2-ylamino)-phenyl]-4-trifluoromethoxy-benzamide;
Compound 113 4-Chloro-N-[4-(4-pyridin-2-yl-pyrimidin-2-ylamino)-phenyl]-benzamide;
Compound 114 2-Methoxy-N-[4-(4-pyridin-2-yl-pyrimidin-2-ylamino)-phenyl]-benzamide;
Compound 115 4-Methyl-N-[4-(4-pyridin-2-yl-pyrimidin-2-ylamino)-phenyl]-benzamide;
Compound 116 Thiophene-2-sulfonic acid [4-(4-pyridin-2-yl-pyrimidin-2-ylamino)-phenyl]-amide;
Compound 117 N-[4-(4-Pyridin-3-yl-pyrimidin-2-ylamino)-phenyl]-4-trifluoromethoxy-benzamide;
Compound 118 N-[4-(4-Pyridin-2-yl-pyrimidin-2-ylamino)-phenyl]-nicotinamide;
Compound 119 Thiophene-2-carboxylic acid [4-(4-pyridin-2-yl-pyrimidin-2-ylamino)-phenyl]-amide;
Compound 120 N-[4-(4-Pyridin-4-yl-pyrimidin-2-ylamino)-phenyl]-benzamide;
Compound 121 N-[4-(4-Pyridin-3-yl-pyrimidin-2-ylamino)-phenyl]-nicotinamide;
Compound 122 4-Methoxy-N-[4-(4-pyridin-3-yl-pyrimidin-2-ylamino)-phenyl]-benzamide;
Compound 123 3,5-Dimethoxy-N-[4-(4-pyridin-2-yl-pyrimidin-2-ylamino)-phenyl]-benzamide;
Compound 124 3,5-Dimethoxy-N-[4-(4-pyridin-3-yl-pyrimidin-2-ylamino)-phenyl]-benzamide;
Compound 125 2-Chloro-N-[4-(4-pyridin-4-yl-pyrimidin-2-ylamino)-phenyl]-acetamide;
Compound 126 2-Chloro-N-[4-(4-pyridin-2-yl-pyrimidin-2-ylamino)-phenyl]-acetamide;
Compound 127 2-Chloro-N-[4-(4-pyridin-3-yl-pyrimidin-2-ylamino)-phenyl]-acetamide;
Compound 128 2-Chloro-N-[3-(4-pyridin-3-yl-pyrimidin-2-ylamino)-phenyl]-acetamide;
Compound 129 2-Chloro-N-[3-(4-pyridin-4-yl-pyrimidin-2-ylamino)-phenyl]-acetamide;
Compound 130 2-Chloro-N-[3-(4-pyridin-2-yl-pyrimidin-2-ylamino)-phenyl]-acetamide;
Compound 131 2-(4-Methyl-piperazin-1-yl)-N-[3-(4-pyridin-3-yl-pyrimidin-2-ylamino)-phenyl]-acetamide;
Compound 132 N*3*-[4-(4-Imidazol-1-yl-phenyl)-pyrimidin-2-yl]-4-methyl-benzene-1,3-diamine;
Compound 133 4-Chloro-N-{3-[4-(4-imidazol-1-yl-phenyl)-pyrimidin-2-ylamino]-4-methyl-phenyl}-benzamide;
Compound 134 N-{3-[4-(4-Imidazol-1-yl-phenyl)-pyrimidin-2-ylamino]-4-methyl-phenyl}-4-methyl-benzamide;
Compound 135 [4-(4-Imidazol-1-yl-phenyl)-pyrimidin-2-yl]-(3-nitro-phenyl)-amine;
Compound 136 N-{3-[4-(4-Imidazol-1-yl-phenyl)-pyrimidin-2-ylamino]-4-methyl-phenyl}-3,4,5-trimethoxy-benzamide;
Compound 137 4-Cyano-N-{3-[4-(4-imidazol-1-yl-phenyl)-pyrimidin-2-ylamino]-4-methyl-phenyl}-benzamide;
Compound 138 N-{3-[4-(4-Imidazol-1-yl-phenyl)-pyrimidin-2-ylamino]-4-methyl-phenyl}-nicotinamide;
Compound 139 Thiophene-2-sulfonic acid {3-[4-(4-imidazol-1-yl-phenyl)-pyrimidin-2-ylamino]-4-methyl-phenyl}-amide;
Compound 140 Naphthalene-2-carboxylic acid {3-[4-(4-imidazol-1-yl-phenyl)-pyrimidin-2-ylamino]-4-methyl-phenyl}-amide;
Compound 141 N-{3-[4-(4-Imidazol-1-yl-phenyl)-pyrimidin-2-ylamino]-4-methyl-phenyl}-2-methoxy-benzamide;
Compound 142 N-[4-(4-Imidazol-1-yl-phenyl)-pyrimidin-2-yl]-benzene-1,3-diamine;
Compound 143 N,N′-Bis-[4-(4-imidazol-1-yl-phenyl)-pyrimidin-2-yl]-benzene-1,3-diamine;
Compound 144 [4-(3,4-Dimethoxy-phenyl)-pyrimidin-2-yl]-(2-methyl-5-nitro-phenyl)-amine;
Compound 145 [4-(3,4-Dimethoxy-phenyl)-pyrimidin-2-yl]-(3-nitro-phenyl)-amine;
Compound 146 4-Chloromethyl-N-{3-[4-(4-imidazol-1-yl-phenyl)-pyrimidin-2-ylamino]-4-methyl-phenyl}-benzamide;
Compound 147 N-[4-(3,4-Dimethoxy-phenyl)-pyrimidin-2-yl]-benzene-1,3-diamine;
Compound 148 4-Cyano-N-{3-[4-(4-imidazol-1-yl-phenyl)-pyrimidin-2-ylamino]-phenyl}-benzamide;
Compound 149 Naphthalene-2-carboxylic acid {3-[4-(4-imidazol-1-yl-phenyl)-pyrimidin-2-ylamino]-phenyl}-amide;
Compound 150 N-{3-[4-(4-Imidazol-1-yl-phenyl)-pyrimidin-2-ylamino]-phenyl}-nicotinamide;
Compound 151 N-{3-[4-(4-Imidazol-1-yl-phenyl)-pyrimidin-2-ylamino]-phenyl}-3,4,5-trimethoxy-benzamide;
Compound 152 4-Chloro-N-{3-[4-(4-imidazol-1-yl-phenyl)-pyrimidin-2-ylamino]-phenyl}-benzamide;
Compound 153 Naphthalene-2-carboxylic acid {3-[4-(3,4-dimethoxy-phenyl)-pyrimidin-2-ylamino]-4-methyl-phenyl}-amide;
Compound 154 N-{3-[4-(3,4-Dimethoxy-phenyl)-pyrimidin-2-ylamino]-4-methyl-phenyl}-4-methyl-benzamide;
Compound 155 4-Chloromethyl-N-{3-[4-(3,4-dimethoxy-phenyl)-pyrimidin-2-ylamino]-4-methyl-phenyl}-benzamide;
Compound 156 N-{3-[4-(3,4-Dimethoxy-phenyl)-pyrimidin-2-ylamino]-4-methyl-phenyl}-4-(4-methyl-piperazin-1-ylmethyl)-benzamide;
Compound 157 N-{3-[4-(4-Imidazol-1-yl-phenyl)-pyrimidin-2-ylamino]-phenyl}-4-methyl-benzamide;
Compound 158 N-{3-[4-(4-Imidazol-1-yl-phenyl)-pyrimidin-2-ylamino]-phenyl}-2-methoxy-benzamide;
Compound 159 N-{3-[4-(3,4-Dihydroxy-phenyl)-pyrimidin-2-ylamino]-4-methyl-phenyl}-4-(4-methyl-piperazin-1-ylmethyl)-benzamide;
Compound 160 4-Chloromethyl-N-{3-[4-(4-imidazol-1-yl-phenyl)-pyrimidin-2-ylamino]-phenyl}-benzamide;
Compound 161 4-Chloro-N-{3-[4-(3,4-dimethoxy-phenyl)-pyrimidin-2-ylamino]-4-methyl-phenyl}-benzamide;
Compound 162 N-{3-[4-(3,4-Dimethoxy-phenyl)-pyrimidin-2-ylamino]-4-methyl-phenyl}-3,4,5-trimethoxy-benzamide;
Compound 163 N-{3-[4-(3,4-Dimethoxy-phenyl)-pyrimidin-2-yl]}-4-methyl-benzene-1,3-diamine;
Compound 164 4-Cyano-N-{3-[4-(3,4-dimethoxy-phenyl)-pyrimidin-2-ylamino]-4-methyl-phenyl}-benzamide;
Compound 165 N-{3-[4-(3,4-Dimethoxy-phenyl)-pyrimidin-2-ylamino]-4-methyl-phenyl}-nicotinamide;
Compound 166 N-{3-[4-(3,4-Dimethoxy-phenyl)-pyrimidin-2-ylamino]-4-methyl-phenyl}-2-methoxy-benzamide;
Compound 167 N-(3-Methyl-isoxazolo[5,4-d]pyrimidin-4-yl)-N′-(4-pyridin-3-yl-pyrimidin-2-yl)-benzene-1,3-diamine;
Compound 168 N-(3-Methyl-isoxazolo[5,4-d]pyrimidin-4-yl)-N′-(4-pyridin-4-yl-pyrimidin-2-yl)-benzene-1,3-diamine;
Compound 169 5-(1-Imino-ethyl)-6-[4-(4-pyridin-4-yl-pyrimidin-2-ylamino)-phenylamino]-3H-pyrimidin-4-one;
Compound 170 N-(3-Methyl-isoxazolo[5,4-d]pyrimidin-4-yl)-N′-(4-pyridin-4-yl-pyrimidin-2-yl)-benzene-1,4-diamine;
Compound 171 2-(4-Methyl-piperazin-1-yl)-N-[4-(4-pyridin-4-yl-pyrimidin-2-ylamino)-phenyl]-acetamide;
Compound 172 4-{[4-(4-Pyridin-4-yl-pyrimidin-2-ylamino)-phenylcarbamoyl]-methyl}-piperazine-1-carboxylic acid ethyl ester;
Compound 173 2-Morpholin-4-yl-N-[4-(4-pyridin-4-yl-pyrimidin-2-ylamino)-phenyl]-acetamide;
Compound 174 1-{[4-(4-Pyridin-4-yl-pyrimidin-2-ylamino)-phenylcarbamoyl]-methyl}-piperidine-4-carboxylic acid ethyl ester;
Compound 175 2-Chloro-N-[4-methyl-3-(4-pyridin-3-yl-pyrimidin-2-ylamino)-phenyl]-acetamide;
Compound 176 4-Methyl-N-[1-(3-methyl-isoxazolo[5,4-d]pyrimidin-4-yl)]-N′-[3-(4-pyridin-3-yl-pyrimidin-2-yl)]-benzene-1,3-diamine;
Compound 177 5-(1-Imino-ethyl)-6-[4-methyl-3-(4-pyridin-3-yl-pyrimidin-2-ylamino)-phenylamino]-pyrimidin-4-ol;
Compound 178 5-(1-Imino-ethyl)-6-[4-methyl-3-(4-pyridin-4-yl-pyrimidin-2-ylamino)-phenylamino]-pyrimidin-4-ol;
Compound 179 [1,8]Naphthyridine-2-carboxylic acid [4-methyl-3-(4-pyridin-3-yl-pyrimidin-2-ylamino)-phenyl]-amide;
Compound 180 2-Chloro-N-[4-methyl-3-(4-pyridin-2-yl-pyrimidin-2-ylamino)-phenyl]-acetamide;
Compound 181 5-(1-Imino-ethyl)-6-[3-(4-pyridin-3-yl-pyrimidin-2-ylamino)-phenylamino]-pyrimidin-4-ol;
Compound 182 4-(4-Methyl-piperazin-1-ylmethyl)-N-[4-(4-pyridin-2-yl-pyrimidin-2-ylamino)-phenyl]-benzamide;
Compound 183 4-{[3-(4-Pyridin-3-yl-pyrimidin-2-ylamino)-phenylcarbamoyl]-methyl}-piperazine-1-carboxylic acid ethyl ester;
Compound 184 2-Morpholin-4-yl-N-[3-(4-pyridin-3-yl-pyrimidin-2-ylamino)-phenyl]-acetamide;
Compound 185 1-{[3-(4-Pyridin-3-yl-pyrimidin-2-ylamino)-phenylcarbamoyl]-methyl}-piperidine-4-carboxylic acid ethyl ester;
Compound 186 2-(4-Methyl-piperazin-1-yl)-N-[4-(4-pyridin-2-yl-pyrimidin-2-ylamino)-phenyl]-acetamide;
Compound 187 2-Morpholin-4-yl-N-[4-(4-pyridin-2-yl-pyrimidin-2-ylamino)-phenyl]-acetamide;
Compound 188 4-{[4-(4-Pyridin-2-yl-pyrimidin-2-ylamino)-phenylcarbamoyl]-methyl}-piperazine-1-carboxylic acid ethyl ester;
Compound 189 1-{[4-(4-Pyridin-2-yl-pyrimidin-2-ylamino)-phenylcarbamoyl]-methyl}-piperidine-4-carboxylic acid ethyl ester;
Compound 190 2-(4-Methyl-piperazin-1-yl)-N-[4-methyl-3-(4-pyridin-3-yl-pyrimidin-2-ylamino)-phenyl]-acetamide;
Compound 191 1-{[4-Methyl-3-(4-pyridin-3-yl-pyrimidin-2-ylamino)-phenylcarbamoyl]-methyl}-piperidine-4-carboxylic acid ethyl ester;
Compound 192 4-{[4-Methyl-3-(4-pyridin-3-yl-pyrimidin-2-ylamino)-phenylcarbamoyl]-methyl}-piperazine-1-carboxylic acid ethyl ester;
Compound 193 N-[4-Methyl-3-(4-pyridin-3-yl-pyrimidin-2-ylamino)-phenyl]-2-morpholin-4-yl-acetamide;
Compound 194 4-Methyl-N-[1-(3-methyl-isoxazolo[5,4-d]pyrimidin-4-yl)]-N′-[3-(4-pyridin-4-yl-pyrimidin-2-yl)]-benzene-1,3-diamine;
Compound 195 2-(4-Methyl-piperazin-1-yl)-N-[4-methyl-3-(4-pyridin-4-yl-pyrimidin-2-ylamino)-phenyl]-acetamide;
Compound 196 N-[4-Methyl-3-(4-pyridin-4-yl-pyrimidin-2-ylamino)-phenyl]-2-morpholin-4-yl-acetamide;
Compound 197 3,4,5-Trimethoxy-N-[4-(4-pyridin-4-yl-pyrimidin-2-ylamino)-phenyl]-benzamide;
Compound 198 2-Methoxy-N-[4-(4-pyridin-4-yl-pyrimidin-2-ylamino)-phenyl]-benzamide;
Compound 199 N-[4-(4-Pyridin-4-yl-pyrimidin-2-ylamino)-phenyl]-nicotinamide;
Compound 200 Naphthalene-2-carboxylic acid [4-(4-pyridin-4-yl-pyrimidin-2-ylamino)-phenyl]-amide;
Compound 201 4-Chloro-N-[4-(4-pyridin-4-yl-pyrimidin-2-ylamino)-phenyl]-benzamide;
Compound 202 1-{[4-Methyl-3-(4-pyridin-4-yl-pyrimidin-2-ylamino)-phenylcarbamoyl]-methyl}-piperidine-4-carboxylic acid ethyl ester;
Compound 203 Thiophene-2-sulfonic acid [4-(4-pyridin-4-yl-pyrimidin-2-ylamino)-phenyl]-amide;
Compound 204 4-{[4-Methyl-3-(4-pyridin-4-yl-pyrimidin-2-ylamino)-phenylcarbamoyl]-methyl}-piperazine-1-carboxylic acid ethyl ester;
Compound 205 4-Bromo-N-[4-(4-pyridin-4-yl-pyrimidin-2-ylamino)-phenyl]-benzamide;
Compound 206 2,3,4,5,6-Pentafluoro-N-[4-(4-pyridin-4-yl-pyrimidin-2-ylamino)-phenyl]-benzamide;
Compound 207 2-(4-Methyl-piperazin-1-yl)-N-[4-methyl-3-(4-pyridin-2-yl-pyrimidin-2-ylamino)-phenyl]-acetamide;
Compound 208 N-[4-Methyl-3-(4-pyridin-2-yl-pyrimidin-2-ylamino)-phenyl]-2-morpholin-4-yl-acetamide;
Compound 209 4-Chloro-N-[4-(4-pyridin-4-yl-pyrimidin-2-ylamino)-phenyl]-benzenesulfonamide;
Compound 210 Naphthalene-2-sulfonic acid [4-(4-pyridin-4-yl-pyrimidin-2-ylamino)-phenyl]-amide;
Compound 211 4-Methyl-N-[3-(4-pyridin-3-yl-pyrimidin-2-ylamino)-phenyl]-benzenesulfonamide;
Compound 212 [1,8]Naphthyridine-2-carboxylic acid [4-methyl-3-(4-pyridin-4-yl-pyrimidin-2-ylamino)-phenyl]-amide;
Compound 213 4-(4-Methyl-piperazin-1-ylmethyl)-N-[4-methyl-3-(4-pyridin-4-yl-pyrimidin-2-ylamino)-phenyl]-benzamide;
Compound 214 1-{[4-Methyl-3-(4-pyridin-2-yl-pyrimidin-2-ylamino)-phenylcarbamoyl]-methyl}-piperidine-4-carboxylic acid ethyl ester;
Compound 215 4-{[4-Methyl-3-(4-pyridin-2-yl-pyrimidin-2-ylamino)-phenylcarbamoyl]-methyl}-piperazine-1-carboxylic acid ethyl ester;
Compound 216 4-Chloro-N-[4-(4-pyridin-2-yl-pyrimidin-2-ylamino)-phenyl]-benzenesulfonamide;
Compound 217 Naphthalene-2-sulfonic acid [4-(4-pyridin-2-yl-pyrimidin-2-ylamino)-phenyl]-amide;
Compound 218 4-Chloro-N-[4-(4-pyridin-3-yl-pyrimidin-2-ylamino)-phenyl]-benzenesulfonamide,
Compound 219 Naphthalene-2-sulfonic acid [4-(4-pyridin-3-yl-pyrimidin-2-ylamino)-phenyl]-amide;
Compound 220 2-Methoxy-N-[4-methyl-3-(4-pyridin-4-yl-pyrimidin-2-ylamino)-phenyl]-benzamide;
Compound 221 N-(3-Methyl-isoxazolo[5,4-d]pyrimidin-4-yl)-N′-(4-pyridin-2-yl-pyrimidin-2-yl)-benzene-1,4-diamine;
Compound 222 Naphthalene-2-carboxylic acid [4-methyl-3-(4-pyridin-4-yl-pyrimidin-2-ylamino)-phenyl]-amide;
Compound 223 N-[4-Methyl-3-(4-pyridin-4-yl-pyrimidin-2-ylamino)-phenyl]-benzamide;
Compound 224 2-(4-Methyl-piperazin-1-yl)-N-[3-(4-pyridin-4-yl-pyrimidin-2-ylamino)-phenyl]-acetamide;
Compound 225 4-Methyl-N-[4-methyl-3-(4-pyridin-4-yl-pyrimidin-2-ylamino)-phenyl]-benzamide;
Compound 226 2-Morpholin-4-yl-N-[3-(4-pyridin-4-yl-pyrimidin-2-ylamino)-phenyl]-acetamide;
Compound 227 Naphthalene-2-sulfonic acid [4-methyl-3-(4-pyridin-4-yl-pyrimidin-2-ylamino)-phenyl]-amide;
Compound 228 1-{[3-(4-Pyridin-4-yl-pyrimidin-2-ylamino)-phenylcarbamoyl]-methyl}-piperidine-4-carboxylic acid ethyl ester;
Compound 229 4-{[3-(4-Pyridin-4-yl-pyrimidin-2-ylamino)-phenylcarbamoyl]-methyl}-piperazine-1-carboxylic acid ethyl ester;
Compound 230 1-{[3-(4-Pyridin-4-yl-pyrimidin-2-ylamino)-phenylcarbamoyl]-methyl}-piperidine-4-carboxylic acid;
Compound 231 Cyclohexanecarboxylic acid [4-methyl-3-(4-pyridin-4-yl-pyrimidin-2-ylamino)-phenyl]-amide;
Compound 232 N,N′-Bis-[4-(3,4-dimethoxy-phenyl)-pyrimidin-2-yl]-benzene-1,3-diamine;
Compound 233 N-[4-(4-Imidazol-1-yl-phenyl)-pyrimidin-2-yl]-N′-(3-methyl-isoxazolo[5,4-d]pyrimidin-4-yl)-benzene-1,3-diamine;
Compound 234 3-Fluoro-N-{3-[4-(4-imidazol-1-yl-phenyl)-pyrimidin-2-ylamino]-phenyl}-benzamide;
Compound 235 N-{3-[4-(3,4-Dimethoxy-phenyl)-pyrimidin-2-ylamino]-4-methyl-phenyl}-3-fluoro-benzamide;
Compound 236 N-{3-[4-(4-Imidazol-1-yl-phenyl)-pyrimidin-2-ylamino]-phenyl}-4-(4-methyl-piperazin-1-ylmethyl)-benzamide;
Compound 237 N-{3-[4-(3,4-Dimethoxy-phenyl)-pyrimidin-2-yl]}-4-methyl-N′-[1-(3-methyl-isoxazolo[5,4-d]pyrimidin-4-yl)]-benzene-1,3-diamine;
Compound 238 3-Fluoro-N-{3-[4-(4-imidazol-1-yl-phenyl)-pyrimidin-2-ylamino]-4-methyl-phenyl}-benzamide;
Compound 239 N-{3-[4-(3,4-Dimethoxy-phenyl)-pyrimidin-2-ylamino]-phenyl}-3-fluoro-benzamide;
Compound 240 N-{3-[4-(4-Imidazol-1-yl-phenyl)-pyrimidin-2-yl]}-4-methyl-N′-[1-(3-methyl-isoxazolo[5,4-d]pyrimidin-4-yl)]-benzene-1,3-diamine;
Compound 241 4-Chloro-N-{3-[4-(3,4-dimethoxy-phenyl)-pyrimidin-2-ylamino]-phenyl}-benzamide;
Compound 242 N-{3-[4-(3,4-Dimethoxy-phenyl)-pyrimidin-2-ylamino]-phenyl}-4-methyl-benzamide;
Compound 243 N-{3-[4-(3,4-Dimethoxy-phenyl)-pyrimidin-2-ylamino]-phenyl}-3,4,5-trimethoxy-benzamide;
Compound 244 4-Cyano-N-{3-[4-(3,4-dimethoxy-phenyl)-pyrimidin-2-ylamino]-phenyl}-benzamide;
Compound 245 4-Chloromethyl-N-{3-[4-(3,4-dimethoxy-phenyl)-pyrimidin-2-ylamino]-phenyl}-benzamide;
Compound 246 Naphthalene-2-carboxylic acid {3-[4-(3,4-dimethoxy-phenyl)-pyrimidin-2-ylamino]-phenyl}-amide;
Compound 247 N-{3-[4-(3,4-Dimethoxy-phenyl)-pyrimidin-2-ylamino]-phenyl}-2-methoxy-benzamide;
Compound 248 N-{3-[4-(3,4-Dimethoxy-phenyl)-pyrimidin-2-ylamino]-phenyl}-nicotinamide;
Compound 249 N-{3-[4-(3,4-Dimethoxy-phenyl)-pyrimidin-2-ylamino]-phenyl}-4-(4-methyl-piperazin-1-ylmethyl)-benzamide;
Compound 250 N-[4-(3,4-Dimethoxy-phenyl)-pyrimidin-2-yl]-N′-(3-methyl-isoxazolo[5,4-d]pyrimidin-4-yl)-benzene-1,3-diamine;
Compound 251 Isoquinoline-5-sulfonic acid {3-[4-(3,4-dimethoxy-phenyl)-pyrimidin-2-ylamino]-4-methyl-phenyl}-amide;
Compound 252 N-{3-[4-(4-Imidazol-1-yl-phenyl)-pyrimidin-2-ylamino]-4-methyl-phenyl}-4-(4-methyl-piperazin-1-ylmethyl)-benzamide;
Compound 253 (4-Benzo[1,3]dioxol-5-yl-pyrimidin-2-yl)-(2-methyl-5-nitro-phenyl)-amine;
Compound 254 4-(4-Methyl-piperazin-1-ylmethyl)-N-[4-(4-pyridin-4-yl-pyrimidin-2-ylamino)-phenyl]-benzamide;
Compound 255 N-[4-(4-Imidazol-1-yl-phenyl)-pyrimidin-2-yl]-benzene-1,4-diamine;
Compound 256 N-[4-(3,4-Dimethoxy-phenyl)-pyrimidin-2-yl]-benzene-1,4-diamine;
Compound 257 Naphthalene-2-carboxylic acid {4-[4-(4-imidazol-1-yl-phenyl)-pyrimidin-2-ylamino]-phenyl}-amide;
Compound 258 (3-Chloro-phenyl)-(4-pyridin-4-yl-pyrimidin-2-yl)-amine;
Compound 259 [1,8]Naphthyridine-2-carbothioic acid [4-methyl-3-(4-pyridin-4-yl-pyrimidin-2-ylamino)-phenyl]-amide;
Compound 260 4-Methyl-N-[4-methyl-3-(4-pyridin-3-yl-pyrimidin-2-ylamino)-phenyl]-benzenesulfonamide;
Compound 261 3-Chloro-N-[4-methyl-3-(4-pyridin-4-yl-pyrimidin-2-ylamino)-phenyl]-propionamide;
Compound 262 3-(4-Methyl-piperazin-1-yl)-N-[4-methyl-3-(4-pyridin-4-yl-pyrimidin-2-ylamino)-phenyl]-propionamide;
Compound 263 4-{2-[4-Methyl-3-(4-pyridin-4-yl-pyrimidin-2-ylamino)-phenylcarbamoyl]-ethyl}-piperazine-1-carboxylic acid ethyl ester;
Compound 264 2-(4-Methyl-piperazin-1-yl)-N-[3-(4-pyridin-2-yl-pyrimidin-2-ylamino)-phenyl]-acetamide;
Compound 265 2-Morpholin-4-yl-N-[3-(4-pyridin-2-yl-pyrimidin-2-ylamino)-phenyl]-acetamide;
Compound 266 N-[4-Methyl-3-(4-pyridin-2-yl-pyrimidin-2-ylamino)-phenyl]-nicotinamide;
Compound 267 1-{[3-(4-Pyridin-2-yl-pyrimidin-2-ylamino)-phenylcarbamoyl]-methyl}-piperidine-4-carboxylic acid ethyl ester;
Compound 268 4-{[3-(4-Pyridin-2-yl-pyrimidin-2-ylamino)-phenylcarbamoyl]-methyl}-piperazine-1-carboxylic acid ethyl ester;
Compound 269 Naphthalene-2-carboxylic acid [4-methyl-3-(4-pyridin-2-yl-pyrimidin-2-ylamino)-phenyl]-amide;
Compound 270 4-Bromo-N-[4-methyl-3-(4-pyridin-2-yl-pyrimidin-2-ylamino)-phenyl]-benzamide;
Compound 271 1-{[3-(4-Pyridin-2-yl-pyrimidin-2-ylamino)-phenylcarbamoyl]-methyl}-piperidine-4-carboxylic acid;
Compound 272 4-Methyl-N-[4-methyl-3-(4-pyridin-2-yl-pyrimidin-2-ylamino)-phenyl]-benzamide;
Compound 273 Naphthalene-2-sulfonic acid [4-methyl-3-(4-pyridin-2-yl-pyrimidin-2-ylamino)-phenyl]-amide;
Compound 274 4-Chloromethyl-N-[4-methyl-3-(4-pyridin-2-yl-pyrimidin-2-ylamino)-phenyl]-benzamide;
Compound 275 2-Methoxy-N-[4-methyl-3-(4-pyridin-2-yl-pyrimidin-2-ylamino)-phenyl]-benzamide;
Compound 276 4-(4-Methyl-piperazin-1-ylmethyl)-N-[4-methyl-3-(4-pyridin-2-yl-pyrimidin-2-ylamino)-phenyl]-benzamide;
Compound 277 4-Fluoro-N-[4-methyl-3-(4-pyridin-2-yl-pyrimidin-2-ylamino)-phenyl]-benzenesulfonamide;
Compound 278 Cyclopentanecarboxylic acid [4-methyl-3-(4-pyridin-2-yl-pyrimidin-2-ylamino)-phenyl]-amide;
Compound 279 3-{4-[2-(3-Chloro-phenylamino)-pyrimidin-4-yl]-pyridin-2-ylamino}-propan-1-ol;
Compound 280 Isoquinoline-5-sulfonic acid {3-[4-(4-imidazol-1-yl-phenyl)-pyrimidin-2-ylamino]-4-methyl-phenyl}-amide;
Compound 281 (2-Methyl-5-nitro-phenyl)-(4-pyridin-3-yl-pyrimidin-2-yl)-amine;
Compound 282 (3-Chloro-phenyl)-[4-(2-chloro-pyridin-4-yl)-pyrimidin-2-yl]-amine;
Compound 283 4-Chloromethyl-N-[3-(4-pyridin-4-yl-pyrimidin-2-ylamino)-phenyl]-benzamide;
Compound 284 Thiophene-2-sulfonic acid {3-[4-(3,4-dimethoxy-phenyl)-pyrimidin-2-ylamino]-4-methyl-phenyl}-amide;
Compound 285 4-(4-Methyl-piperazin-1-ylmethyl)-N-[3-(4-pyridin-4-yl-pyrimidin-2-ylamino)-phenyl]-benzamide;
Compound 286 N-{1-{5-[2-(3,4,5-Trimethoxy-phenylamino)-pyrimidin-4-yl]-pyridin-2-yl}}-ethane-1,2-diamine;
Compound 287 [4-(6-Dimethylamino-pyridin-3-yl)-pyrimidin-2-yl]-(3,4,5-trimethoxy-phenyl)-amine;
Compound 288 2,2-Dimethyl-N-{3-[2-(2-methyl-5-nitro-phenylamino)-pyrimidin-4-yl]-pyridin-4-yl}-propionamide;
Compound 289 [4-(4-Amino-pyridin-3-yl)-pyrimidin-2-yl]-(2-methyl-5-nitro-phenyl)-amine;
Compound 290 3-{5-[2-(3-Chloro-phenylamino)-pyrimidin-4-yl]-pyridin-2-ylamino}-propan-1-ol;
Compound 291 (3-Chloro-phenyl)-[4-(6-chloro-pyridin-3-yl)-pyrimidin-2-yl]-amine;
Compound 294 [4-(1-Oxy-pyridin-4-yl)-pyrimidin-2-yl]-(3-trifluoromethyl-phenyl)-amine;
Compound 295 3-(4-Pyridin-4-yl-pyrimidin-2-ylamino)-benzoic acid ethyl ester;
Compound 296 3-[4-(1-Oxy-pyridin-4-yl)-pyrimidin-2-ylamino]-benzoic acid ethyl ester;
Compound 297 3-{4-[2-(3-Trifluoromethyl-phenylamino)-pyrimidin-4-yl]-pyridin-2-ylamino}-propan-1-ol;
Compound 298 2-{4-[2-(3-Trifluoromethyl-phenylamino)-pyrimidin-4-yl]-pyridin-2-ylamino}-ethanol;
Compound 299 5-{4-[2-(3-Trifluoromethyl-phenylamino)-pyrimidin-4-yl]-pyridin-2-ylamino}-pentan-1-ol;
Compound 300 {4-[2-(3-Imidazol-1-yl-propylamino)-pyridin-4-yl]-pyrimidin-2-yl}-(3-trifluoromethyl-phenyl)-amine;
Compound 301 (4-{2-[3-(4-Methyl-piperazin-1-yl)-propylamino]-pyridin-4-yl}-pyrimidin-2-yl)-(3-trifluoromethyl-phenyl)-amine;
Compound 302 3-(4-Pyridin-4-yl-pyrimidin-2-ylamino)-benzoic acid;
Compound 303 N-(3-Hydroxy-propyl)-3-{4-[2-(3-hydroxy-propylamino)-pyridin-4-yl]-pyrimidin-2-ylamino}-benzamide;
Compound 304 3-[4-(2-Chloro-pyridin-4-yl)-pyrimidin-2-ylamino]-benzoic acid;
Compound 305 3-{4-[2-(3-Hydroxy-propylamino)-pyridin-4-yl]-pyrimidin-2-ylamino}-benzoic acid;
Compound 306 1-[3-(4-Pyridin-4-yl-pyrimidin-2-ylamino)-benzoyl]-piperidine-4-carboxylic acid ethyl ester;
Compound 307 3-{4-[2-(3-Hydroxy-propylamino)-pyridin-4-yl]-pyrimidin-2-ylamino}-benzoic acid methyl ester;
Compound 308 N-(4,4-Diethoxy-butyl)-3-(4-pyridin-4-yl-pyrimidin-2-ylamino)-benzamide;
Compound 309 [4-(2-Chloro-pyridin-4-yl)-pyrimidin-2-yl]-(3-methylsulfanyl-phenyl)-amine;
Compound 310 2-{4-[2-(3-Methylsulfanyl-phenylamino)-pyrimidin-4-yl]-pyridin-2-ylamino}-ethanol;
Compound 311 5-{4-[2-(3-Methylsulfanyl-phenylamino)-pyrimidin-4-yl]-pyridin-2-ylamino}-pentan-1-ol; and
Compound 312 3-{4-[2-(3-Methylsulfanyl-phenylamino)-pyrimidin-4-yl]-pyridin-2-ylamino}-propan-1-ol;

7. (canceled)

8. A method for inhibiting the production of a kinase and/or a phosphatase in an individual in need thereof, comprising administering to the individual a pharmaceutical composition comprising a compound according to claim 1, wherein said individual is suffering from a disease or disorder which can be cured or relieved by the inhibition of said kinase and/or phosphatase.

9. The method according to claim 8, wherein the kinase and/or phosphatase is selected from the group consisting of Abl, Akt, c-kit, EGF-R, GSK3β, JNK, Lck, PDGF-Rβ, PknG, and ROCK2.

10. A method for treating an individual suffering from a disease or disorder selected from the group consisting of cell proliferation disorders, cancer, leukemia, erectile dysfunction, cardiovascular diseases and disorders, inflammatory diseases, transplant rejection, immunological diseases, neuroimmunological diseases, autoimmune diseases, infective diseases including opportunistic infections, prion diseases, neurodegenerative disorders and/or neuro-degeneration, the method comprising administering to said individual a pharmaceutical composition comprising a compound according to claim 1 or a pharmaceutically-acceptable salt thereof.

11. The method according to claim 10, wherein the proliferation disorder or cancer is selected from the group consisting of adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gallbladder cancer, gall bladder carcinomas, uterine cancer, cervical cancer, glioblastomas, gynecologic tumors, ear, nose and throat tumors, hematologic neoplasias, hairy cell leukemia, urethral cancer, skin cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma, and tongue cancer.

12. The method according to claim 10, wherein the cardiovascular disease or disorder is selected from the group consisting of aneurysm, stable angina, unstable angina, angina pectoris, angioneurotic edema, stenosis, restenosis, aortic valve stenosis, aortic aneurysm, arrhythmia, arrhythmogenic right ventricular dysplasia, arteriosclerosis, arteriovenous malformations, atrial fibrillation, Behcet Syndrome, bradycardia, cardiac tamponade, cardiomegaly, congestive cardiomyopathy, hypertrophic cardiomyopathy, restrictive cardiomyopathy, carotid stenosis, cerebral hemorrhage, Churg-Strauss Syndrome, diabetes, Ebstein's Anomaly, Eisenmenger Complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau Disease, hyperemia, hypertension, pulmonary hypertension, left ventricular hypertrophy, right ventricular hypertrophy, hypoplastic left heart syndrome, hypotension, intermittent claudication, ischemic heart disease, Klippel-Trenaunay-Weber Syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud Disease, Sneddon Syndrome, superior vena cava syndrome, syndrome X, tachycardia, Takayasu's Arteritis, hereditary hemorrhagic telangiectasia, telangiectasis, temporal Arteritis, tetralogy of fallot, thromboangiitis obliterans, thrombosis, thromboembolism, tricuspid atresia, varicose veins, vascular diseases, vasculitis, vasospasm, ventricular fibrillation, Williams Syndrome, peripheral vascular disease, varicose veins and leg ulcers, deep vein thrombosis, and Wolff-Parkinson-White Syndrome.

13. The method according to claim 10, wherein the inflammatory disease is selected from the group consisting of diseases which are associated with overexpression or overproduction of the protein amyloid A, including arthritides, rheumatoid arthritis, asthma, lupus, bleeding disorders (thrombocytopenia), chronic inflammatory lung diseases, atherosclerosis, kidney inflammation (nephritis), psoriasis, allergies, Crohn's disease, ischemia/reperfusion injury, endotoxemic liver injury, inflammatory bowel disease, tuberculosis, chronic infections, familial Mediterranean fever, interstitial cystitis and skin sunburn.

14. The method according to claim 10, wherein the neurodegenerative disorders or neurodegeneration is selected from the group consisting of Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA).

15. The method according to claim 10, wherein the immunological disease, neuroimmunological disease, and/or autoimmune disease is selected from the group consisting of asthma, diabetes, rheumatic diseases, AIDS, rejection of transplanted organs and tissues, rhinitis, chronic obstructive pulmonary diseases, osteoporisis, ulcerative colitis, sinusitis, lupus erythematosus, recurrent infections, atopic dermatitis/eczema and occupational allergies, food allergies, drug allergies, severe anaphylactic reactions, anaphylaxis, manifestations of allergic diseases, primary immunodeficiencies, antibody deficiency states, cell mediated immunodeficiencies, severe combined immunodeficiency, DiGeorge syndrome, Hyper-IgE syndrome, Wiskott-Aldrich syndrome, ataxia-telangiectasia, immune mediated cancers, white cell defects, autoimmune diseases, systemic lupus erythematosus, rheumatoid arthritis (RA), multiple sclerosis (MS), immune-mediated or Type 1 Diabetes Mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, Hashimoto's disease, dermatomyositis, goodpastture syndrome, myasthenia gravis pseudoparalytica, ophtalmia sympatica, phakogene uveitis, chronical aggressive hepatitis, primary billiary cirrhosis, autoimunehemolytic anemy, and Werlof disease.

16. The method according to claim 10, wherein the infective disease including opportunistic infection is selected from the group consisting of AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever.

17. The method according to claim 10, wherein the prion diseases is selected from the group consisting of Scrapie, TME, CWD, BSE, CJD, vCJD, GSS, FFI, Kuru, and Alpers Syndrome.

18. The method according to claim 10, wherein the transplant rejection is selected from the group consisting of heart transplant rejection, heart-lung transplant rejection, lung transplant rejection, liver transplant rejection, kidney transplant rejection, pancreas transplant rejection, spleen transplant rejection, skin transplant rejection, tissue transplant rejection, bone marrow transplant rejection, spinal marrow transplant rejection, hormone producing glands transplant rejection, gonads and gonadal gland transplant rejection, graft-versus-host-diseases and host-versus-graft-diseases.

19. A pharmaceutical composition comprising at least one compound according to claim 1 and a pharmaceutically acceptable carrier, excipient, or diluent.

20. The pharmaceutical composition according to claim 19, wherein the pharmaceutical composition is formulated as pills, tablets, tabs, film tablets, coated tablets, dragees, multi-layer tablets, capsules, powders, granulates, deposits, sustained release formulations, controlled release formulations, mini- and micro-formulations, nano-formulations, liposomal formulations, dispersions, suspensions, liquid formulations, drops, injections, sprays, ointments, creams, pastes, syrup, lotions, or gels.

21. A method for detecting prion infections and/or prion diseases in a sample from an individual comprising:
a) providing a sample from an individual; and
b) adding to said sample at least one compound according to claim 1 or a pharmaceutically active salt thereof; and
c) detecting activity in said sample of the human cellular protein kinase Abl.
